(19) United States
(12) Patent Application Publication
(10) Pub. No.: US 2003/0165911 A1
(43) Pub. Date: Sep. 4, 2003

(54) Gene expression analysis using nicking agents

(75) Inventors: Jeffrey Van Ness, Claremont, CA (US); David J. Galas, Claremont, CA (US); Lori K. Van Ness, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Keck Graduate Institute, Claremont, CA (US)

(21) Appl. No.: 10/196,350

(22) Filed: Jul. 15, 2002

Related U.S. Application Data
(60) Provisional application No. 60/331,687, filed on Nov. 19, 2001.
(60) Provisional application No. 60/305,637, filed on Jul. 15, 2001.
(60) Provisional application No. 60/345,445, filed on Jan. 2, 2002.

(51) Int. Cl. C12Q 1/68; C12P 19/34

(52) U.S. Cl. 435/6; 435/91.2

(57) ABSTRACT

The present invention provides methods and compositions for gene expression analyses using nicking agents.

### BACKGROUND OF THE INVENTION

1. Field of the Invention

This invention relates to the field of molecule biology, more particularly to methods and compositions involving nucleic acids and still more particularly to methods and compositions related to gene expression analysis using nicking agents.

2. Description of the Related Art

Gene expression analyses are important to identify genes that are involved in diseases and in growth and development of organisms. To increase the sensitivity of such analyses, cDNA molecules may be amplified before being detected or quantified. A number of nucleic acid amplification methods may be used to amplify cDNA, such as polymerase chain reaction (PCR), ligase chain reaction (LCR) and strand displacement amplification (SDA). Most of the methods widely used for nucleic acid amplification, such as PCR, require cycles of different temperatures to achieve cycles of denaturation and reannealing. Other methods, although they may be performed isothermally, require multiple sets of primers (e.g., bumper primers of thermophilic SDA). Accordingly, there is a long felt need in the art for a simpler and more efficient method for amplifying cDNA to increase the sensitivity of gene expression analyses.

The present invention fulfills this and related needs as described below.

### BRIEF SUMMARY OF THE INVENTION

In contrast to currently available methods for amplifying nucleic acids such as cDNA molecules, the present invention provides a method for nucleic acid amplification that does not require the use of multiple sets of oligonucleotide primers. In addition, the present invention can be carried out under isothermal conditions, thus avoiding the expenses associated with the equipment for providing cycles of different temperatures.

In one aspect, the present invention provides a method for determining the presence or absence of a target cDNA molecule in a cDNA population or for determining the presence or absence of a target mRNA molecule in a biological sample, comprising:

(A) forming a mixture comprising:

(i) the cDNA molecules of the cDNA population or the RNA molecules of the biological sample,

(ii) a template nucleic acid molecule that

(a) comprises one strand of a nicking agent recognition sequence, and

(b) is at least substantially complementary to the target cDNA if the target cDNA is single-stranded,

is at least substantially complementary to one strand of the target cDNA if the target cDNA is double-stranded, or

is at least substantially complementary to the target mRNA,

(iii) a nicking agent that recognizes the recognition sequence,

(iv) a DNA polymerase, and

(v) one or more deoxynucleoside triphosphate(s);

(B) maintaining the mixture at conditions that amplify a single-stranded nucleic acid molecule using a portion of the target cDNA, a portion of the target mRNA, or a portion of the template nucleic acid molecule as a template, if the target cDNA is present in the cDNA population or if the target mRNA is present in the biological sample; and

(C) detecting the presence or absence of the single-stranded nucleic acid molecule to determine the presence or absence of the target cDNA molecule in the cDNA population, or to determine the presence or absence of the target mRNA in the biological sample.

In certain embodiments, the template nucleic acid comprises a sequence, located 3′ to the sequence of the one strand of the nicking agent recognition sequence, that is at least substantially complementary to the 3′ portion of the target cDNA if the target cDNA is single-stranded, to the 3′ portion of one strand of the target cDNA if the target cDNA is double-stranded, or to the target mRNA.

In some embodiments, the target cDNA is double-stranded and comprises the nicking agent recognition sequence, and wherein the template nucleic acid comprises the portion of the target cDNA that contains the sequence of the antisense strand of the nicking agent recognition sequence.

In other embodiments, the target cDNA is single-stranded and comprises the sequence of the sense strand of the nicking agent recognition sequence, and wherein the template nucleic acid comprises the sequence of the antisense strand of the nicking agent recognition sequence.

In some embodiments, the target cDNA is double-stranded and comprises the nicking agent recognition sequence, and wherein the template nucleic acid comprises, from 3′ to 5′:

(i) a sequence that is at least substantially complementary to the strand of the target cDNA that comprises the sequence of the sense strand of the nicking agent recognition sequence,

(ii) the sequence of the antisense strand of the nicking agent recognition sequence, and

(iii) a sequence that is not substantially complementary to the strand of the target cDNA that comprises the sequence of the sense strand of the nicking agent recognition sequence.

In certain embodiments, the target cDNA is single-stranded and comprises the sequence of the sense strand of the nicking agent recognition sequence, and wherein the template nucleic acid comprises, from 3′ to 5′:

(i) a sequence that is at least substantially complementary to the target cDNA,

(ii) the sequence of the antisense strand of the nicking agent recognition sequence, and

(iii) a sequence that is not substantially complementary to the target cDNA.

In some embodiments, the template nucleic acid molecule comprises the sequence of the sense strand of the nicking agent recognition sequence. In such embodiments, one or more nucleotides in the sequence of the sense strand of the nicking agent recognition sequence may or may not form a conventional base pair with nucleotides of the target cDNA or the target mRNA.

In other embodiments, the template nucleic acid molecule comprises the sequence of the antisense strand of the nicking agent recognition sequence.

In another aspect, the present invention provides a method for determining the presence or absence of an mRNA in a sample, comprising:

(a) synthesizing single-stranded cDNA molecules using the mRNA molecules in the sample as templates;

(b) forming a mixture comprising:

(i) the single-strand cDNA molecules from step (a),

(ii) a single-stranded nucleic acid probe that comprises, from 3′ to 5′, a sequence that is at least substantially complementary to the 3′ portion of the target nucleic acid, and a sequence of the antisense strand of a nicking agent recognition sequence;

(c) removing unhybridized probe from the mixture of step (b);

(d) performing an amplification reaction in the presence of a nicking agent that recognizes the nicking agent recognition sequence; and

(e) detecting and/or characterizing the presence or absence of the amplification product of step (d) to determine the presence or absence of the target nucleic acid in the sample.

In some embodiments, the 5′ termini of the single-stranded cDNA molecules are immobilized, such as via the use of an immobilized oligonucleotide primer.

In another aspect, the present invention provides a method for determining the presence or absence of a double-stranded target cDNA molecule that comprises a nicking agent recognition sequence in a cDNA population, comprising:

(A) forming a mixture comprising the cDNA population, a nicking agent that recognizes the nicking agent recognition sequence, a DNA polymerase, and one or more deoxynucleoside triphosphate(s);

(B) maintaining the mixture at conditions that amplify a single-stranded nucleic acid molecule using one strand of the target cDNA molecule as a template, if the target cDNA molecule is present in the cDNA population; and

(C) detecting the presence or absence of the single-stranded nucleic acid fragment amplified in step (B) to determine the presence or absence of the target cDNA.

In another aspect, the present invention provides a method for profiling a cDNA population comprising:

(A) forming a mixture comprising the cDNA population, a nicking agent, a DNA polymerase, and one or more deoxynucleoside triphosphate(s);

(B) maintaining the mixture at conditions that amplify single-stranded nucleic acid molecules using the cDNA molecules that comprise a recognition sequence of the nicking agent as templates; and

(C) characterizing the single-stranded nucleic acid fragments to profile the cDNA population.

In another aspect, the present invention provides a method for determining the presence or absence of a target cDNA molecule in a cDNA population, or for determining the presence or absence of a target mRNA in a biological sample, comprising

(A) forming a mixture comprising:

(i) the cDNA molecules in the cDNA population, or the RNA molecules of the biological sample;

(ii) a partially double-stranded nucleic acid probe that comprise:

(a) a sequence of a sense strand of a nicking agent recognition sequence, a sequence of an antisense strand of the nicking agent recognition sequence, or both; and

(b) a 5′ overhang in the strand that either the strand itself or an extension product thereof contains a nicking site nickable by a nicking agent that recognizes the nicking agent recognition sequence, or

a 3′ overhang in the strand that either the strand nor an extension product thereof contains the nicking site,

wherein each overhang comprises a nucleic acid sequence at least substantially complementary to the target cDNA if the target cDNA is single-stranded, to one strand of the target cDNA if the target cDNA is double-stranded, or to the target mRNA;

(B) separating the probe molecules that have hybridized to the cDNA or mRNA molecules from those that have not;

(C) performing an amplification reaction in the presence of the hybridized probe molecules and a nicking agent that recognizes the nicking agent recognition sequence to amplify a single-stranded nucleic acid fragment using one strand of the partially double-stranded nucleic acid probe as a template, if the target cDNA is present in the cDNA population or if the target mRNA is present in the biological sample; and

(D) detecting the presence or absence of the single-stranded nucleic acid fragment of step (C) to determine the presence or absence of the target cDNA in the cDNA population, or to determine the presence or absence of the target mRNA in the biological sample.

In another aspect, the present invention provides a method for determining the presence or absence of a target cDNA molecule in a cDNA population, comprising

(A) forming a mixture of a first oligonucleotide primer (ODNP), a second ODNP, and the cDNA molecules in the cDNA population, wherein

(i) if the target cDNA is a double-stranded nucleic acid having a first strand and a second strand,

the first ODNP comprises a nucleotide sequence of a sense strand of a nicking endonuclease recognition sequence and a nucleotide sequence at least substantially complementary to a first portion of the first strand of the target nucleic acid, and

the second ODNP comprises a nucleotide sequence at least substantially complementary to a second portion of the second strand of the target nucleic acid and comprises a sequence of one strand of a restriction endonuclease recognition sequence, the second portion being located 3′ to the complement of the first portion in the second strand of the target nucleic acid,

or

(ii) if the target nucleic acid is a single-stranded nucleic acid,

the first ODNP comprises a nucleotide sequence of a sense strand of a nicking endonuclease recognition sequence and a nucleotide sequence at least substantially identical to a first portion of the target nucleic acid, and

the second ODNP comprises a nucleotide sequence at least substantially complementary to a second portion of the target nucleic acid and comprises a sequence of one strand of a restriction endonuclease recognition sequence, the second portion being located 5′ to the first portion in the target nucleic acid;

(B) subjecting the mixture to conditions that, if the target cDNA is present in the cDNA population,

(i) extend the first and the second ODNPs to produce an extension product comprising the first ODNP and the second ODNP;

(ii) optionally digesting the extension product of step (i) with a restriction endonuclease that recognizes the restriction endoculease recognition sequence to provide a digestion product;

(iii) amplify a single-stranded nucleic acid fragment using one strand of the extension product of step (B)(i) or the digestion product of step (B)(ii) as a template in the presence of a nicking endonuclease that recognizes the nicking endonuclease recognition sequence; and

(C) detecting the presence or absence of the single-stranded nucleic acid fragment of step (B)(ii) to determine the presence or absence of the target cDNA in the cDNA population.

In another aspect, the present invention provides a method for determining the presence or absence of a target cDNA in a cDNA population, comprising

(A) forming a mixture of a first oligonucleotide primer (ODNP), a second ODNP, and the cDNA molecules of the cDNA population, wherein

(i) if the target cDNA is a double-stranded nucleic acid having a first strand and a second strand,

the first ODNP comprises a nucleotide sequence of a sense strand of a first nicking endonuclease recognition sequence (NERS) and a nucleotide sequence at least substantially complementary to a first portion of the first strand of the target cDNA, and

the second ODNP comprises a nucleotide sequence at least substantially complementary to a second portion of the second strand of the target nucleic acid and comprises a sequence of the sense strand of a second NERS, the second portion being located 3′ to the complement of the first portion in the second strand of the target cDNA,

or

(ii) if the target cDNA is a single-stranded nucleic acid,

the first ODNP comprises a nucleotide sequence of a sense strand of a first NERS and a nucleotide sequence at least substantially identical to a first portion of the target cDNA, and

the second ODNP comprises a nucleotide sequence at least substantially complementary to a second portion of the target nucleic acid and comprises a sequence of the sense strand of a second NERS, the second portion being located 5′ to the first portion in the target cDNA;

(B) subjecting the mixture to conditions that, if the target cDNA is present in the cDNA population,

(i) extend the first and the second ODNPs to produce an extension product comprising both the first and the second NERSs;

(ii) amplify a single-stranded nucleic acid fragment using one strand of the extension product of step (B)(i) as a template in the presence of one or more nicking endonucleases (NEs) that recognizes the first and the second NERSs; and

(C) detecting the presence or absence of the single-stranded nucleic acid fragment of step (B)(ii) to determine the presence or absence of the target nucleic acid in the sample.

In another aspect, the present invention provides a method for determining the presence or absence of a target cDNA in a cDNA population, comprising

(A) forming a mixture of a first oligonucleotide primer (ODNP), a second ODNP, and the cDNA molecules of the cDNA population, wherein

(i) if the target cDNA is a double-stranded nucleic acid having a first strand and a second strand,

the first ODNP comprises a nucleotide sequence of a sense strand of a restriction endonuclease recognition sequence (RERS) and a nucleotide sequence at least substantially complementary to a first portion of the first strand of the target cDNA, and

the second ODNP comprises a nucleotide sequence at least substantially complementary to a second portion of the second strand of the target nucleic acid and comprises a sequence of the sense strand of a second RERS, the second portion being located 3′ to the complement of the first portion in the second strand of the target cDNA,

or

(ii) if the target cDNA is a single-stranded nucleic acid,

the first ODNP comprises a nucleotide sequence of a sense strand of a first RERS and a nucleotide sequence at least substantially identical to a first portion of the target cDNA, and

the second ODNP comprises a nucleotide sequence at least substantially complementary to a second portion of the target nucleic acid and comprises a sequence of the sense strand of a second RERS, the second portion being located 5′ to the first portion in the target cDNA;

(B) subjecting the mixture to conditions that, if the target cDNA is present in the cDNA population,

(i) extend the first and the second ODNPs to produce an extension product comprising both the first and the second RERSs;

(ii) amplify a single-stranded nucleic acid fragment using one strand of the extension product of step (B)(i) as a template in the presence of one more restriction endonucleases (REs) that recognizes the first and the second RERSs; and

(C) detecting the presence or absence of the single-stranded nucleic acid fragment of step (B)(ii) to determine the presence or absence of the target cDNA in the cDNA population.

In another aspect, the present invention provides a method for determining the presence or absence of a target cDNA molecule in a cDNA population, or for determining the presence or absence of a target mRNA molecule in a biological sample, comprising:

(A) forming a mixture comprising:

(i) the cDNA molecules of the cDNA population, or the RNA molecule of the biological sample,

(ii) a first single-stranded template nucleic acid molecule (T1) that

(a) comprises one strand of a first nicking agent recognition sequence, and

(b) is at least substantially complementary to the target cDNA if the target cDNA is single-stranded,

is at least substantially complementary to one strand of the target cDNA if the target cDNA is double-stranded, or

is at least substantially complementary to the target mRNA,

(iii) a first nicking agent that recognizes the first nicking agent recognition sequence,

(iv) a DNA polymerase, and

(v) one or more deoxynucleoside triphosphate(s);

(B) maintaining the mixture at conditions that amplify a first single-stranded nucleic acid molecule (A1 ) using a portion of the target cDNA, a portion of the target mRNA, or a portion of the template nucleic acid molecule as a template, if the target cDNA is present in the cDNA population or if the target mRNA is present in the biological sample;

(C) providing a second single-stranded template nucleic acid molecule (T2 ) that is at least substantially complementary to A1 and comprises one strand of a second nicking agent recognition sequence;

(D) performing an amplification reaction in the presence of a second nicking agent that recognizes the second nicking agent recognition sequence to amplify a second single-stranded nucleic acid molecule (A2 ) using either A1 or T2 as a template; and

(E) detecting the presence or absence of A2 to determine the presence or absence of the target cDNA molecule in the cDNA population or the presence or absence of the target mRNA in the biological sample.

In some embodiments, the first template nucleic acid is single-stranded and comprises a sequence, located 3′ to the sequence of one strand of the first nicking agent recognition sequence, that is at least substantially complementary to the 3′ portion of the target cDNA if the target cDNA is single-stranded to one strand of the target cDNA if the target cDNA is double-stranded, or to the target mRNA.

In some embodiments, the target cDNA is double-stranded and comprises the first nicking agent recognition sequence, and wherein the first template nucleic acid comprises the portion of the target cDNA that contains the sequence of the antisense strand of the first nicking agent recognition sequence.

In some embodiments, the target cDNA is single-stranded and comprises the sequence of the sense strand of the first nicking agent recognition sequence, and wherein the first template nucleic acid molecule comprises the sequence of the antisense strand of the first nicking agent recognition sequence.

In certain embodiments, the target cDNA is double-stranded and comprises the first nicking agent recognition sequence, and wherein the first template nucleic acid comprises, from 3′ to 5′:

(i) a sequence that is at least substantially complementary to the strand of the target cDNA that comprises the sequence of the sense strand of the first nicking agent recognition sequence,

(ii) the sequence of the antisense strand of the first nicking agent recognition sequence, and

(iii) a sequence that is not substantially complementary to the strand of the target cDNA that comprises the sequence of the sense strand of the first nicking agent recognition sequence.

In certain embodiments, the target cDNA is single-stranded and comprises the sequence of the sense strand of the first nicking agent recognition sequence, and wherein the first template nucleic acid comprises, from 3′ to 5′:

(i) a sequence that is at least substantially complementary to the target cDNA,

(ii) the sequence of the antisense strand of the first nicking agent recognition sequence, and

(iii) a sequence that is not substantially complementary to the target cDNA.

In another aspect, the present invention provides a method for determining the presence or absence of a target cDNA molecule in a cDNA population, comprising:

(A) forming a mixture comprising:

(i) the cDNA molecules of the cDNA population,

(ii) a first single-stranded template nucleic acid molecule (T1 ) that

(a) comprises a sequence of the antisense strand of a first nicking agent recognition sequence, and

(b) is at least substantially complementary to the target cDNA if the target cDNA is single-stranded, or

is at least substantially complementary to one strand of the target cDNA if the target cDNA is double-stranded,

(iii) a second single-stranded template nucleic acid molecule (T2 ) that comprises, from 3′ to 5′:

(a) a sequence that is at least substantially identical to the sequence of the T1 located 5′ to the sequence of the antisense strand of the first nicking agent recognition sequence, and

(b) a sequence of the antisense strand of a second nicking agent recognition sequence,

(iv) a first nicking agent that recognizes the first nicking agent recognition sequence,

(v) a second nicking agent that recognizes the second nicking agent recognition sequence,

(vi) a DNA polymerase, and

(vii) one or more deoxynucleoside triphosphate(s);

(B) maintaining the mixture at conditions that amplify a first single-stranded nucleic acid molecule (A2 ) using the T2 as a template, if the target cDNA is present in the cDNA population; and

(C) detecting the presence or absence of A2 to determine the presence or absence of the target cDNA molecule in the cDNA population.

In another aspect, the present invention provides a method for determining the presence or absence of a target cDNA molecule in a cDNA population, comprising:

(A) forming a mixture comprising:

(i) the cDNA molecules of the cDNA population,

(ii) a first single-stranded template nucleic acid molecule (T1 ) that

(a) comprises a sequence of the sense strand of a first nicking agent recognition sequence, and

(b) is at least substantially complementary to the target cDNA if the target cDNA is single-stranded, or

is at least substantially complementary to one strand of the target cDNA if the target cDNA is double-stranded,

(iii) a second single-stranded template nucleic acid molecule (T2 ) that comprises, from 3′ to 5′:

(a) a sequence that is at least substantially complementary to the sequence of T1 located 3′ to the sequence of the sense strand of the first nicking agent recognition sequence, and

(b) a sequence of the antisense strand of a second nicking agent recognition sequence,

(iv) a first nicking agent that recognizes the first nicking agent recognition sequence,

(V) a second nicking agent that recognizes the second nicking agent recognition sequence,

(vi) a DNA polymerase, and

(vii) one or more deoxynucleoside triphosphate(s);

(B) maintaining the mixture at conditions that amplify a first single-stranded nucleic acid molecule (A2 ) using T2 as a template, if the target cDNA is present in the cDNA population; and

(C) detecting the presence or absence of A2 to determine the presence or absence of the target cDNA molecule in the cDNA population.

In another aspect, the present invention provides a method for determining the presence or absence of a target cDNA molecule in a cDNA population, comprising:

(A) forming a mixture comprising:

(i) the cDNA molecules of the cDNA population,

(ii) a first single-stranded template nucleic acid molecule (T1 ) that

(a) comprises a sequence of the antisense strand of a first nicking agent recognition sequence, and

(b) is at least substantially complementary to the target cDNA if the target cDNA is single-stranded, or

is at least substantially complementary to one strand of the target cDNA if the target cDNA is double-stranded,

(iii) a second single-stranded template nucleic acid molecule (T2 ) that comprises, from 3′ to 5′:

(a) a sequence that is at least substantially identical to the sequence of T1 located 5′ to the sequence of the antisense strand of the first nicking agent recognition sequence, and

(b) a sequence of the sense strand of a second nicking agent recognition sequence,

(iv) a first nicking agent that recognizes the first nicking agent recognition sequence,

(V) a second nicking agent that recognizes the second nicking agent recognition sequence,

(vi) a DNA polymerase, and

(vii) one or more deoxynucleoside triphosphate(s);

(B) maintaining the mixture at conditions that amplify a first single-stranded nucleic acid molecule (A2 ) that is at least substantially identical to the sequence of T1 located 5′ to the antisense strand of the first nicking agent recognition sequence, if the target cDNA is present in the cDNA population; and

(C) detecting the presence or absence of A2 to determine the presence or absence of the target cDNA molecule in the cDNA population.

In another aspect, the present invention provides a method for determining the presence or absence of a target cDNA molecule in a cDNA population, comprising:

(A) forming a mixture comprising:

(i) the cDNA molecules of the cDNA population,

(ii) a first single-stranded template nucleic acid molecule (T1 ) that

(a) comprises a sequence of the sense strand of a first nicking agent recognition sequence, and

(b) is at least substantially complementary to the target cDNA if the target cDNA is single-stranded, or

is at least substantially complementary to one strand of the target cDNA if the target cDNA is double-stranded,

(iii) a second single-stranded template nucleic acid molecule (T2 ) that comprises, from 3′ to 5′:

(a) a sequence that is at least substantially complementary to the sequence of T1 located 3′ to the sequence of the sense strand of the first nicking agent recognition sequence, and

(b) a sequence of the sense strand of a second nicking agent recognition sequence,

(iv) a first nicking agent that recognizes the first nicking agent recognition sequence,

(v) a second nicking agent that recognizes the second nicking agent recognition sequence,

(vi) a DNA polymerase, and

(vii) one or more deoxynucleoside triphosphate(s);

(B) maintaining the mixture at conditions that amplify a first single-stranded nucleic acid molecule (A2 ) that is at least substantially identical to the sequence of T1 located 3′ to the sense strand of the first nicking agent recognition sequence, if the target cDNA is present in the cDNA population; and

(C) detecting the presence or absence of A2 to determine the presence or absence of the target cDNA molecule in the cDNA population.

In another aspect, the present invention provides a method for determining the presence or absence of a target cDNA molecule in a cDNA population, or for determining the presence or absence of a target mRNA molecule in a biological sample, comprising:

(A) forming a mixture comprising:

(i) the cDNA molecules of the cDNA population, or the RNA molecule of the biological sample,

(ii) a first template nucleic acid molecule (T1 ) that comprises, from 3′ to 5′:

(a) a first sequence that is at least substantially complementary to the 3′ portion of the target cDNA if the target cDNA is single-stranded, or

a first sequence that is at least substantially complementary to the 3′ portion of one strand of the target cDNA if the target cDNA is double-stranded, or

a first sequence that is at least substantially complementary to the 3′ portion of the target mRNA,

(b) a sequence of the antisense strand of a first nicking agent recognition sequence, and

(c) a second sequence,

(iii) a second template nucleic acid molecule (T2 ) comprising, from 3′ to 55′:

(a) a first sequence that is at least substantially identical to the second sequence of T1 ,

(b) a sequence of the antisense strand of a second nicking agent recognition sequence, and

(c) a second sequence,

(iv) a first nicking agent that recognizes the first nicking agent recognition sequence,

(v) a second nicking agent that recognizes the second nicking agent recognition sequence,

(vi) a DNA polymerase, and

(vii) one or more deoxynucleoside triphosphate(s);

(B) maintaining the mixture at conditions that amplify a single-stranded nucleic acid molecule (A2 ) using the second sequence of T2 as a template, if the target cDNA is present in the cDNA population; and

(C) detecting the presence or absence of A2 to determine the presence or absence of the target cDNA molecule in the cDNA population or the presence or absence of the target mRNA in the biological sample.

In another aspect, the present invention provides a method for determining the presence or absence of a target cDNA molecule that comprises a sequence of a sense strand of a first nicking agent recognition sequence in a cDNA population, comprising:

(A) forming a mixture comprising:

(i) the cDNA molecules of the cDNA population,

(ii) a first template nucleic acid molecule (T1 ) that comprises, from 3′ to 55′:

(a) a first sequence that is at least substantially complementary to the portion of the target cDNA located immediately 5′ to the sequence of the sense strand of the first nicking agent recognition sequence,

(b) a sequence of the antisense strand of a first nicking agent recognition sequence, and

(c) a second sequence,

(iii) a second template nucleic acid molecule (T2 ) comprising, from 3′ to 55′:

(a) a first sequence that is at least substantially identical to the second sequence of T1 ,

(b) a sequence of the antisense strand of a second nicking agent recognition sequence, and

(c) a second sequence,

(iv) a first nicking agent that recognizes the first nicking agent recognition sequence,

(v) a second nicking agent that recognizes the second nicking agent recognition sequence,

(vi) a DNA polymerase, and

(vii) one or more deoxynucleoside triphosphate(s);

(B) maintaining the mixture at conditions that amplify a single-stranded nucleic acid molecule (A2 ) using the second sequence of T2 as a template, if the target cDNA is present in the cDNA population; and

(C) detecting the presence or absence of A2 to determine the presence or absence of the target cDNA molecule in the cDNA population.

In another aspect, the present invention provides a nucleic acid comprising a sequence that is at least substantially identical to a portion of a naturally occurring genomic DNA or a cDNA of a naturally occurring mRNA, wherein

(A) the portion of the naturally occurring genomic DNA or the cDNA of the naturally occurring mRNA consists of, from 3′ to 55′:

(i) a first sequence that is 3-50 nucleotides in length,

(ii) a sequence of the antisense strand of a nicking agent recognition sequence, and

(iii) a second sequence that is 8-50 nucleotides in length.

(B) the nucleic acid is at most 120 nucleotides in length; and

(C) the nucleic acid comprises sequence A(ii).

In another aspect, the present invention provides a single-stranded nucleic acid that

(a) is at most 100 nucleotides in length,

(b) comprises a sequence of the antisense strand of a nicking agent recognition sequence,

(c) is substantially complementary to a cDNA molecule, and

(d) is capable of functioning as a template to amplify a single-stranded nucleic acid fragment in the presence of a nicking agent that recognizes the nicking agent recognition sequence.

In a related aspect, the present invention provides a single-stranded nucleic acid that

(a) is at most 100 nucleotides in length,

(b) comprises a sequence of the sense strand of a nicking agent recognition sequence,

(c) is substantially complementary to a cDNA molecule, and

(d) when annealing to the cDNA molecule, allows for the amplification of a portion of the cDNA molecule in the presence of a nicking agent that recognizes the nicking agent recognition sequence.

In another aspect, the present invention provides a method for determining the presence or absence of a target cDNA molecule in a cDNA population, comprising:

(A) forming a mixture comprising:

(i) the cDNA molecules of the cDNA population;

(ii) an oligonucleotide primer that

(a) comprises a sequence of the sense strand of a double-stranded nicking agent recognition sequence recognizable by a nicking agent that nicks outside the recognition sequence, and

(b) is at least substantially complementary to a first region of the single-stranded target nucleic acid or of one strand of the double-stranded target nucleic acid; and

(iii) a partially double-stranded nucleic acid that

(a) comprises a double-stranded type IIs restriction endonucelase recognition sequence, and

(b) a 3′ overhang that is at least substantially complementary to a second region of the single-stranded target cDNA or of the one strand of the double-stranded target cDNA located 5′ to the first region the single-stranded target cDNA or of the one strand of the double-stranded target cDNA;

under conditions that allow for hybridization between the oligonucleotide primer and the first region of the single-stranded target cDNA or of the one strand of the double-stranded nucleic acid and between the 3′ overhang of the partially double-stranded nucleic acid and the second region of the single-stranded target cDNA or of the one strand of the double-stranded nucleic acid;

(B) digesting the single-stranded target cDNA or the one strand of the double-stranded target cDNA that have hybridized to the oligonucleotide primer and to the partially double-stranded nucleic acid in the second region.

(C) performing an amplification reaction that amplify a single-stranded nucleic acid molecule using a portion of the single-stranded target cDNA or of the one strand of the double-stranded target cDNA digested in step (B) as a template in the presence of the nicking agent, and

(D) detecting the presence or absence of the single-stranded nucleic acid molecule of step (C) to determine the presence or absence of the target cDNA molecule in the cDNA population.

These and other aspects of the present invention will become evident upon reference to the following detailed description and attached drawings.

### BRIEF DESCRIPTION OF THE SEVERAL VIEWS OF THE DRAWINGS

FIG. 1 is a schematic diagram of the major steps of a general method for gene expression analysis that performs a linear nucleic acid amplification reaction.

FIG. 2 is a schematic diagram of the major steps of an exemplary method for gene expression analysis that performs a linear nucleic acid amplification reaction. The template nucleic acid molecule T1 comprises the sequence of the antisense strand of the recognition sequence of N.BstNB I.

FIG. 3 is a schematic diagram of the major steps of an exemplary method for gene expression analysis that performs a linear nucleic acid amplification reaction. The template nucleic acid molecule T1 comprises the sequence of the sense strand of the recognition sequence of N.BstNB I.

FIG. 4 is a schematic diagram of the major steps of an exemplary method for gene expression analysis that performs a linear nucleic acid amplification reaction. The target cDNA comprises a restriction endonuclease recognition sequence.

FIG. 5 is a schematic diagram of the major steps of an exemplary method for gene expression analysis that performs a linear nucleic acid amplification reaction. The target cDNA comprises a double-stranded nicking agent recognition sequence. The template nucleic acid molecule T1 is a portion of one strand of the target cDNA that comprises the sequence of the antisense strand of the nicking agent recognition sequence.

FIG. 6 is a schematic diagram of the major steps of an exemplary method for gene expression analysis that performs a linear nucleic acid amplification reaction. The target cDNA comprises a double-stranded nicking agent recognition sequence. The template nucleic acid molecule T1 is at least substantially complementary to the first strand of the target cDNA in Regions X and Y of the T1 molecule, but not substantially complementary to the first strand of the target cDNA in Region Z of the T1 molecule.

FIG. 7 is a schematic diagram of the major steps of an exemplary method for gene expression analysis that performs a linear nucleic acid amplification reaction. The target cDNA is immobilized via its 5′ terminus.

FIG. 8 is a schematic diagram of the major steps of an exemplary method for gene expression analysis that performs a linear nucleic acid amplification reaction. The target cDNA comprises a double-stranded nicking endonuclease recognition sequence and a restriction endonuclease recognition sequence.

FIG. 9 is a schematic diagram of the major steps of an exemplary method for gene expression analysis that performs a linear nucleic acid amplification reaction and uses a partially double-stranded initial nucleic acid molecule N1 that comprises a nicking agent recognition sequence. The target nucleic acid (cDNA or mRNA) is immobilized to a solid support. A nicking endonuclease recognition sequence that is recognizable by a nicking endonuclease that nicks outside its recognition sequence (e.g., N.BstNB I) is used as an exemplary nicking agent recognition sequence.

FIG. 10 is a schematic diagram of the major steps of an exemplary method for gene expression analysis that performs a linear nucleic acid amplification reaction and uses two oligonucleotide primers in preparing an initial nucleic acid molecule N1 . One primer comprises a sequence of the sense strand of a nicking endonuclease recognition sequence while the other comprises a sequence of one strand of a type IIs restriction endonuclease recognition sequence (TRERS).

FIG. 11 is a schematic diagram of the major steps of an exemplary method for gene expression analysis that performs a linear nucleic acid amplification reaction and uses two oligonucleotide primers in preparing an initial nucleic acid molecule N1 . Both primers comprise a sequence of the sense strand of a nicking endonuclease recognition sequence.

FIG. 12 is a schematic diagram of the major steps of an exemplary method for gene expression analysis that performs a linear nucleic acid amplification reaction and uses two oligonucleotide primers in preparing an initial nucleic acid molecule N1 . Both primer comprises a sequence of the sense strand of a hemimodified restriction endonuclease recognition sequence.

FIG. 13 is a schematic diagram of a partial process for gene expression analysis that performs exponential nucleic acid amplification. Only the second amplification reaction of the exponential nucleic acid amplification is illustrated.

FIG. 14 is a schematic diagram of the major steps of an exemplary method for gene expression analysis that performs exponential nucleic acid amplification. The recognition sequence of N.BstNB I is used as an exemplary nicking agent recognition sequence. Both the first template T1 and the second template T2 comprise the sequence of the antisense strand of the recognition sequence of N.BstNB I.

FIG. 15 is a schematic diagram of the major steps of an exemplary method for gene expression analysis that performs exponential nucleic acid amplification. The recognition sequence of N.BstNB I is used as an exemplary nicking agent recognition sequence. The first template T1 comprises the sequence of the sense strand of the recognition sequence of N.BstNB I, while the second template T2 comprises the sequence of the antisense strand of the recognition sequence of N.BstNB I.

FIG. 16 is a schematic diagram of the major steps of an exemplary method for gene expression analysis that performs exponential nucleic acid amplification. The recognition sequence of N.BstNB I is used as an exemplary nicking agent recognition sequence. The first template T1 comprises the sequence of the antisense strand of the recognition sequence of N.BstNB I, while the second template T2 comprises the sequence of the sense strand of the recognition sequence of N.BstNB I.

FIG. 17 is a schematic diagram of the major steps of an exemplary method for gene expression analysis that performs exponential nucleic acid amplification. The recognition sequence of N.BstNB I is used as an exemplary nicking agent recognition sequence. Both the first template T1 and the second template T2 comprise the sequence of the sense strand of the recognition sequence of N.BstNB I.

FIG. 18 shows mass spectrometry analyses of an amplified DNA fragment. The top panel shows the ion current for a fragment with a mass/charge ratio of 1448.6. The middle panel shows the trace from the diode array. The bottom panel shows the total ion current from the mass spectrometer.

FIG. 19 shows mass spectrometry analyses in a control experiment. The top panel shows the trace from the diode array. The top panel shows the total ion current from the mass spectrometer. The middle panel shows the ion current for a fragment with a mass/charge ratio of 1448.6. The bottom panel shows the trace of diode array.

FIG. 20 shows the accumulation of fluorescence of a representative nucleic acid amplification reaction mixture as a function of time.

FIG. 21 shows a schematic diagram of a method for amplifying a single-stranded nucleic acid molecule using an oligonucleotide primer that comprises a sequence of the sense strand of a nicking agent recognition sequence.

FIG. 22 shows a schematic diagram of a method for amplifying a single-stranded nucleic acid molecule using an oligonucleotide primer that comprises a sequence of the sense strand of a nicking agent recognition sequence and a partially double-stranded nucleic acid molecule that comprise a double-stranded type IIs restriction endonuclease recognition sequence (TRERS).

FIG. 23 shows a shematic diagram of the major steps of an exemplary method of exponential amplification of a trigger ODNP, where only one template (T1 ) is used and the recognition sequence of N.BstNB I is used as an exemplary NARS.

### DETAILED DESCRIPTION OF THE INVENTION

The present invention provides methods, compositions and kits for gene expression analyses, such as determining the presence or absence of a target cDNA in a cDNA population or a target mRNA in a biological sample. According to the present invention, the presence of a target cDNA triggers a reaction that linearly or exponentially amplifies a single-strand nucleic acid molecule. The detection of the single-stranded nucleic acid molecule indicates the presence of the target cDNA in the cDNA population or the presence of the target mRNA in the biological sample. Because the present method uses the nucleic acid amplification reaction, it is sensitive in detecting low levels of gene expression.

A. Definitions

Prior to providing a more detailed description of the present invention, it may be helpful to an understanding thereof to define conventions and provide definitions as used herein, as follows. Additional definitions are also provided throughout the description of the present invention.

The terms “3′” and “5′” are used herein to describe the location of a particular site within a single strand of nucleic acid. When a location in a nucleic acid is “3′ to” or “3′ of” a reference nucleotide or a reference nucleotide sequence, this means that the location is between the 3′ terminus of the reference nucleotide or the reference nucleotide sequence and the 3′ hydroxyl of that strand of the nucleic acid. Likewise, when a location in a nucleic acid is “5′ to” or “5′ of” a reference nucleotide or a reference nucleotide sequence, this means that it is between the 5′ terminus of the reference nucleotide or the reference nucleotide sequence and the 5′ phosphate of that strand of the nucleic acid. Further, when a nucleotide sequence is “directly 3′ to” or “directly 3′ of” a reference nucleotide or a reference nucleotide sequence, this means that the nucleotide sequence is immediately next to the 3′ terminus of the reference nucleotide or the reference nucleotide sequence. Similarly, when a nucleotide sequence is “directly 5′ to” or “directly 5′ of ” a reference nucleotide or a reference nucleotide sequence, this means that the nucleotide sequence is immediately next to the 5′ terminus of the reference nucleotide or the reference nucleotide sequence.

A “3′ portion of a single-stranded nucleic acid” refers to a portion of the nucleic acid that contains the 3′ terminus of the nucleic acid. Likewise, a “5′ portion of a single-stranded nucleic acid” refers to a portion of the nucleic acid that contains the 5′ terminus of the nucleic acid.

A “3′ portion of one strand of a double-stranded nucleic acid” refers to a portion of that strand of the nucleic acid that contains the 3′ terminus of that strand of the nucleic acid. Likewise, a “5′ portion of one strand of a double-stranded nucleic acid” refers to a portion of that strand of the nucleic acid that contains the 5′ terminus of that strand of the nucleic acid.

A “naturally occurring genomic DNA” and a “naturally occurring cDNA” refer to a genomic DNA molecule and a cDNA molecule that exist in nature, respectively, no matter whether they are in a purified or non-purified form.

As used herein, “nicking” refers to the cleavage of only one strand of a fully double-stranded nucleic acid molecule or a double-stranded portion of a partially double-stranded nucleic acid molecule at a specific position relative to a nucleotide sequence that is recognized by the enzyme that performs the nicking. The specific position where the nucleic acid is nicked is referred to as the “nicking site” (NS).

A “nicking agent” (NA) is an enzyme that recognizes a particular nucleotide sequence of a completely or partially double-stranded nucleic acid molecule and cleaves only one strand of the nucleic acid molecule at a specific position relative to the recognition sequence. Nicking agents include, but are not limited to, a nicking endonuclease (e.g., N.BstNB I) and a restriction endonuclease (e.g., Hinc II) when a completely or partially double-stranded nucleic acid molecule contains a hemimodified recognition/cleavage sequence in which one strand contains at least one derivatized nucleotide(s) that prevents cleavage of that strand (i.e., the strand that contains the derivatized nucleotide(s)) by the restriction endonuclease.

A “nicking endonuclease” (NE), as used herein, refers to an endonuclease that recognizes a nucleotide sequence of a completely or partially double-stranded nucleic acid molecule and cleaves only one strand of the nucleic acid molecule at a specific location relative to the recognition sequence. Unlike a restriction endonuclease (RE), which requires its recognition sequence to be modified by containing at least one derivatized nucleotide to prevent cleavage of the derivatized nucleotide-containing strand of a fully or partially double-stranded nucleic acid molecule, a NE typically recognizes a nucleotide sequence composed of only native nucleotides and cleaves only one strand of a fully or partially double-stranded nucleic acid molecule that contains the nucleotide sequence.

As used herein, “native nucleotide” refers to adenylic acid, guanylic acid, cytidylic acid, thymidylic acid or uridylic acid. A “derivatized nucleotide” is a nucleotide other than a native nucleotide.

The nucleotide sequence of a completely or partially double-stranded nucleic acid molecule that a NA recognizes is referred to as the “nicking agent recognition sequence” (NARS). Likewise, the nucleotide sequence of a completely or partially double-stranded nucleic acid molecule that a NE recognizes is referred to as the “nicking endonuclease recognition sequence” (NERS). The specific sequence that a RE recognizes is referred to as the “restriction endonuclease recognition sequence” (RERS). A “hemimodified RERS,” as used herein, refers to a double-stranded RERS in which one strand of the recognition sequence contains at least one derivatized nucleotide (e.g., a-thio deoxynucleotide) that prevents cleavage of that strand (i.e., the strand that contains the derivatized nucleotide within the recognition sequence) by a RE that recognizes the RERS.

In certain embodiments, a NARS is a double-stranded nucleotide sequence where each nucleotide in one strand of the sequence is complementary to the nucleotide at its corresponding position in the other strand. In such embodiments, the sequence of a NARS in the strand containing a NS nickable by a NA that recognizes the NARS is referred to as a “sequence of the sense strand of the NARS” or a “sequence of the sense strand of the double-stranded NARS,” while the sequence of the NARS in the strand that does not contain the NS is referred to as a “sequence of the antisense strand of the NARS” or a “sequence of the antisense strand of the double-stranded NARS.”

Likewise, in the embodiments where a NERS is a double-stranded nucleotide sequence of which one strand is exactly complementary to the other strand, the sequence of a NERS located in the strand containing a NS nickable by a NE that recognizes the NERS is referred to as a “sequence of a sense strand of the NERS” or a “sequence of the sense strand of the double-stranded NERS,” while the sequence of the NERS located in the strand that does not contain the NS is referred to a “sequence of the antisense strand of the NERS” or a “sequence of the antisense strand of the double-stranded NERS.” For example, the recognition sequence and the nicking site of an exemplary nicking endonuclease, N.BstNB I, are shown below with “▾” to indicate the cleavage site and N to indicate any nucleotide:

▾
5′-GAGTCNNNNN-3′
3′-CTCAGNNNNN-5′

The sequence of the sense strand of the N.BstNB I recognition sequence is 5′-GAGTC-3′, whereas that of the antisense strand is 5′-GACTC-3′.

Similarly, the sequence of a hemimodified RERS in the strand containing a NS nickable by a RE that recognizes the hemimodified RERS (i.e., the strand that does not contain any derivatized nucleotides) is referred to as “the sequence of the sense strand of the hemimodified RERS” and is located in “the sense strand of the hemimodified RERS” of a hemimodified RERS-containing nucleic acid, while the sequence of the hemimodified RERS in the strand that does not contain the NS (i.e., the strand that contains derivatized nucleotide(s)) is referred to as “the sequence of the antisense strand of the hemimodified RERS” and is located in “the antisense strand of the hemimodified RERS” of a hemimodified RERS-containing nucleic acid.

In certain other embodiments, a NARS is an at most partially double-stranded nucleotide sequence that has one or more nucleotide mismatches, but contains an intact sense strand of a double-stranded NARS as described above. According to the convention used herein, in the context of describing a NARS, when two nucleic acid molecules anneal to one another so as to form a hybridized product, and the hybridized product includes a NARS, and there is at least one mismatched base pair within the NARS of the hybridized product, then this NARS is considered to be only partially double-stranded. Such NARSs may be recognized by certain nicking agents (e.g., N.BstNB I) that require only one strand of double-stranded recognition sequences for their nicking activities. For instance, the NARS of N.BstNB I may contain, in certain embodiments, an intact sense strand, as follows,

5′-GAGTC-3′

3′-NNNNN-5′

where N indicates any nucleotide, and N at one position may or may not be identical to N at another position, however there is at least one mismatched base pair within this recognition sequence. In this situation, the NARS will be characterized as having at least one mismatched nucleotide.

In certain other embodiments, a NARS is a partially or completely single-stranded nucleotide sequence that has one or more unmatched nucleotides, but contains an intact sense strand of a double-stranded NARS as described above. According to the convention used herein, in the context of describing a NARS, when two nucleic acid molecules (i.e., a first and a second strand) anneal to one another so as to form a hybridized product, and the hybridized product includes a nucleotide sequence in the first strand that is recognized by a NA, i.e., the hybridized product contains a NARS, and at least one nucleotide in the sequence recognized by the NA does not correspond to, i.e., is not across from, a nucleotide in the second strand when the hybridized product is formed, then there is at least one unmatched nucleotide within the NARS of the hybridized product, and this NARS is considered to be partially or completely single-stranded. Such NARSs may be recognized by certain nicking agents (e.g., N.BstNB I) that require only one strand of double-stranded recognition sequences for their nicking activities. For instance, the NARS of N.BstNB I may contain, in certain embodiments, an intact sense strand, as follows,

5′-GAGTC-3′

3′-N₀₋₄-5′

(where “N” indicates any nucleotide, 0-4 indicates the number of the nucleotides “N,” a “N” at one position may or may not be identical to a “N” at another position), which contains the sequence of the sense strand of the double-stranded recognition sequence of N.BstNB I. In this instance, at least one of G, A, G, T or C is unmatched, in that there is no corresponding nucleotide in the complementary strand. This situation arises, e.g., when there is a “loop” in the hybridized product, and particularly when the sense sequence is present, completely or in part, within a loop.

As used herein, the phrase “amplifying a nucleic acid molecule” or “amplification of a nucleic acid molecule” refers to the making of two or more copies of the particular nucleic acid molecule. “Exponentially amplifying a nucleic acid molecule” or “exponential amplification of a nucleic acid molecule” refers to the amplification of the particular nucleic acid molecule by a tandem amplification system that comprises two or more nucleic acid amplification reactions. In such a system, the amplification product from the first amplification reaction functions as at least an initial amplification primer for the second nucleic acid amplification reaction. In other words, the amplification product from the first amplification reaction functions at least as a primer during an initial primer extension, but may or may not function as a primer during subsequent primer extensions. As used herein, the term “nucleic acid amplification reaction” refers to the process of making more than one copy of a nucleic acid molecule (A) using a nucleic acid molecule (T) that comprises a sequence complementary to the sequence of nucleic acid molecule A as a template. According to the present invention, both the first and the second nucleic acid amplification reactions employ nicking and primer extension reactions.

An “initial amplification primer,” as used herein, is a primer that anneals to a template nucleic acid and initiates a nucleic acid amplification reaction. An initial primer must function as a primer for an initial primer extension, but need not be the primer for any subsequent primer extensions. For instance, assume that a primer A1 anneals to a portion of a template nucleic acid T2 that comprises the sequence of a sense strand of a NARS at a location 3′ to the sense strand of the NARS. In the presence of a DNA polymerase, the 3′ terminus of A1 is extended using T2 as a template to produce a double-stranded or partially double-stranded nucleic acid molecule (H2) that contains the double-stranded NARS. In the presence of a NA that recognizes the NARS, H2 is nicked in the strand complementary to the initial primer A1 . The strand that contains the 3′ terminus at the nicking site, not the initial primer A1 , may function as a primer for subsequent primer extensions in the presence of the NA and the DNA polymerase. A1 is regarded as an initial primer although it functions as a primer only for the first primer extension, but not the subsequent primer extensions.

A first nucleic acid molecule (“first nucleic acid”) is “derived from” or “originates from” another nucleic acid molecule (“second nucleic acid”) if the first nucleic acid is either a digestion product of the second nucleic acid, or an amplification product using a portion of the second nucleic acid molecule or the complement thereof as a template. The first nucleic acid molecule must comprise a sequence that is exactly identical to, or exactly complementary to, at least a portion of the second nucleic acid.

A first nucleic acid sequence is “at least substantially identical” to a second nucleic acid sequence when the complement of the first sequence is able to anneal to the second sequence in a given reaction mixture (e.g., a nucleic acid amplification mixture). In certain preferred embodiments, the first sequence is “exactly identical” to the second sequence, that is, the nucleotide of the first sequence at each position is identical to the nucleotide of the second sequence at the same position, and the first sequence is of the same length as the second sequence.

A first nucleic acid sequence is “at least substantially complementary” to a second nucleic acid sequence when the first sequence is able to anneal to the second sequence in a given reaction mixture (e.g., a nucleic acid amplification mixture). In certain preferred embodiments, the first sequence is “exactly or completely complementary” to the second sequence, that is, each nucleotide of the first sequence is complementary to the nucleotide of the second sequence at its corresponding position, and the first sequence is of the same length as the second sequence.

As used herein, a nucleotide in one strand (referred to as the “first strand”) of a double-stranded nucleic acid located at a position “corresponding to” another position (e.g., a defined position) in the other strand (referred to as the “second strand”) of a double-stranded nucleic acid refers to the nucleotide in the first strand that is complementary to the nucleotide at the corresponding position in the second strand. Likewise, a position in one strand (referred to as the “first strand”) of a double-stranded nucleic acid corresponding to a nicking site within the other strand (referred to as the “second strand”) of a double-stranded nucleic acid refers to the position between the two nucleotides in the first strand complementary to those in the second strand between which nicking occurs.

“Profiling a cDNA population” refers to the characterization of one or more single-stranded nucleic acid molecules that are amplified using one or more cDNA molecules in the cDNA population as templates. Such a characterization may indicate the presence or absence of certain cDNAs in the cDNA population. It may also be useful in comparing one cDNA population with another cDNA population.

A “cDNA population” refers to a composition that comprises one or more cDNA molecules. The cDNA molecules may be substantially purified so that there is at most minimum amount of molecules other than cDNA molecules present in the composition. In other words, the cDNA population comprises primarily cDNA molecules. Alternatively, the cDNA molecules in a cDNA population may be partially purified so that at least some molecules other than cDNA molecules are removed from the cDNA population. In certain embodiments, the cDNA molecules in a cDNA population may not be purified. In other words, the cDNA population is essentially identical to the biological sample from which the cDNA population is obtained.

B. Gene Expression Analyses Using Linear Nucleic Acid Amplification Methods

In one aspect, the present invention provides a method for gene expression analyses using a linear nucleic acid amplification reaction in the presence of a nicking agent. The method of the present invention may be used to determine the presence or absence of a target cDNA in a cDNA population or the presence or absence of a target mRNA in a biological sample, as well as to profile a cDNA population.

1. Overview

According to the present invention, the presence of a target cDNA in a cDNA population allows for the generation of a fully or partially double-stranded nucleic acid molecule (“an initial nucleic acid molecule (H2)”) that comprises a nicking agent recognition sequence and at least a portion of the target cDNA molecule. In the presence of a nicking agent that recognizes the recognition sequence in the N1 molecule and a DNA polymerase, a single-stranded nucleic acid molecule (A1 ) may be amplified using a portion of the N1 molecule as a template. The detection of the A1 molecule indicates the presence of the target cDNA in the cDNA population. In certain embodiments, a target cDNA itself comprises a nicking agent recognition sequence, thus may function as an initial nucleic acid (N1) molecule. However, if a target cDNA is absent in a cDNA population, no initial nucleic acid (N1) molecule that comprises at least a portion of the target cDNA will be generated. Thus, no single-stranded nucleic acid molecule using a portion of the initial nucleic acid molecule as a template will be amplified. Accordingly, the failure in detecting such a single-stranded nucleic acid molecule may indicate the absence of the target cDNA in the cDNA population.

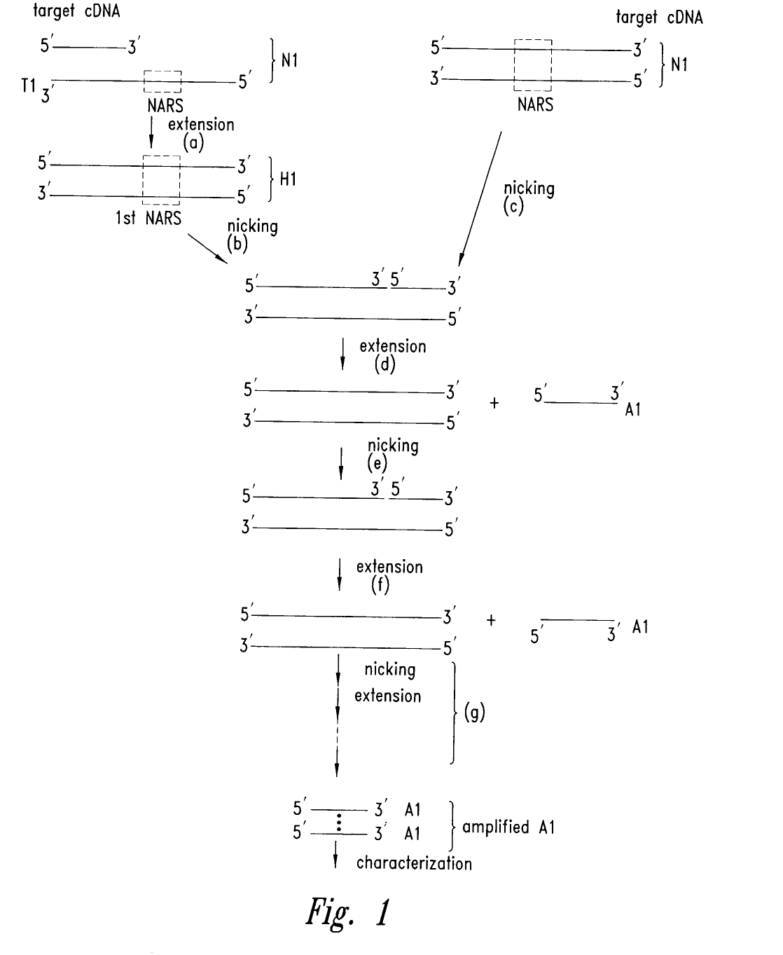

The major steps of an exemplary embodiment are illustrated in FIG. 1. In this embodiment, a template nucleic acid (T1 ) is added to a cDNA population to detect whether the cDNA population contains a target cDNA. The T1 molecule is at least substantially complementary to the target cDNA and comprises a sequence of one strand of a nicking agent recognition sequence. If the target cDNA is present in the cDNA population, it anneals to the T1 molecule to form a partially double-stranded nucleic acid (N1). In the presence of a DNA polymerase, one or both of the 3′ termini of the N1 molecule are extended to form a fully double-stranded nucleic acid molecule (H1) that comprises both strands of the nicking agent recognition sequence (step (a)). In the presence of a nicking agent that recognizes the nicking agent recognition sequence in the H1 molecule, H1 is nicked, producing a 3′ terminus and a 5′ terminus at the nicking site (step (b)). If the fragment containing the 5′ terminus at the nicking site is sufficiently short (e.g., less than 17 nucleotides in length), it will dissociate from the other portion of H1 under certain reaction conditions (e.g., at 60° C.). However, if this fragment does not readily dissociate, it may be displaced by the extension of the fragment containing the 3′ terminus at the nicking site in the presence of a DNA polymerase that is 5′→3′ exonuclease deficient and has a strand displacement activity (step (d)). Strand displacement may also occur in the absence of strand displacement activity in the DNA polymerase, if a strand displacement facilitator is present. Such extension recreates a new nicking site for the nicking agent that can be re-nicked (step (e)). The fragment containing the 5′ terminus at the new nicking site (A1 ) may again readily dissociated from the other portion of H1 or be displaced by extension from the 3′ terminus at the new nicking site (step (f)). The nicking-extension cycles can be repeated multiple times (step (g)), resulting in the linear accumulation of the nucleic acid fragment A1 .

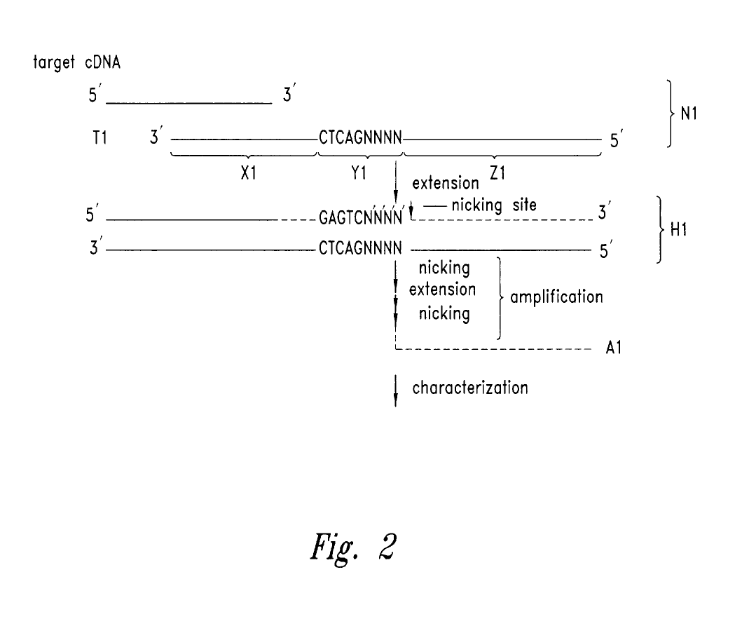

As noted above, a T1 molecule comprises a sequence of one strand of a nicking agent recognition sequence. In certain embodiments, a T1 molecule may comprise a sequence of the antisense strand of a nicking agent recognition sequence. An example of such embodiments is shown in FIG. 2 using the recognition sequence of N.BstNB I as an exemplary nicking agent recognition sequence. In this figure, the initial nucleic acid molecule N1 is a partially double-stranded nucleic acid molecule formed by annealing a single-stranded target cDNA (or one strand of a double-stranded target cDNA) or a portion thereof with a T1 that has three regions: Regions X1, Y1 and Z1. Regions X1, Y1 and Z1 are defined as the region directly 3′ to the sequence of the antisense strand of the N.BstNB I recognition sequence, the region from the 3′ terminus of the sequence of the antisense strand of the recognition sequence of N.BstNB I to the nucleotide corresponding to the 3′ terminal nucleotide at the nicking site of N.BstNB I within the extension product of the trigger ODNP (i.e., 3′-CACAGNNNN-5′ where N can be A, T, G or C), and the region directly 5′ to Region Y1, respectively. The target cDNA is at least substantially complementary to Region X1 and functions as a primer for nucleic acid extension in the presence of a DNA polymerase. The resulting extension product (H1) comprises the double-stranded N.BstNB I recognition sequence and can be nicked by N.BstNB I. The nicked product comprising the sequence of the trigger ODNP may be extended again from its 3′ terminus at the nicking site by the DNA polymerase, which displaces the strand containing the 5′ terminus produced by N.BstNB I at the nicking site. The nicking-extension cycle is repeated multiple times, accumulating the displaced strand (A1) that is exactly complementary to Region Z1.

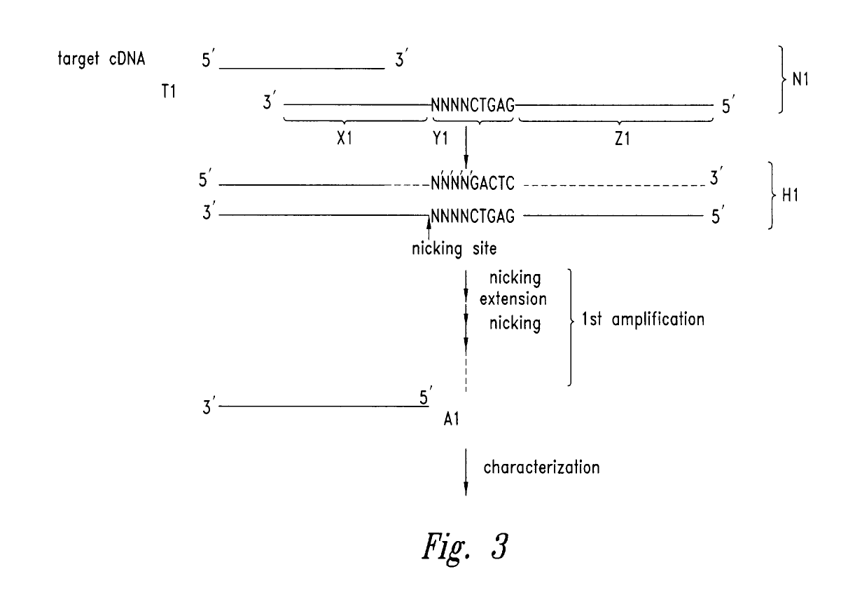

In certain other embodiments, a T1 molecule may comprise a sequence of the sense strand of a nicking agent recognition sequence. An example of such embodiments is shown in FIG. 3 using the recognition sequence of N.BstNB I as an exemplary nicking agent recognition sequence. In this figure, the initial nucleic acid molecule N1 is a partially double-stranded nucleic acid molecule formed by annealing a single-stranded target cDNA (or one strand of a double-stranded target cDNA) or a portion thereof with a T1 having three regions: Regions X1, Y1 and Z1. Regions X1, Y1 and Z1 are defined as the region directly 3′ to the nicking site of the extension product of N1 (i.e., H1 ) by N.BstNB I, the region from the nicking site to the 5′ terminus of the sequence of the sense strand of the recognition sequence of N.BstNB I (i.e., 5′-GAGTCNNNN-3′ where N can be A, T, G or C), and the region directly 5′ to Region Y2, respectively. The target cDNA is at least substantially complementary to Region X1 and functions as a primer for nucleic acid extension in the presence of a DNA polymerase. The resulting extension product (H1) comprises the double-stranded N.BstNB I recognition sequence and can be nicked by N.BstNB I. The nicked product comprising the sequence of the sense strand of the recognition sequence of N.BstNB I may be extended again from its 3′ terminus at the nicking site by the DNA polymerase, which displaces the strand containing the 5′ terminus produced by N.BstNB I at the nicking site. The nicking-extension cycle is repeated multiple times, resulting in the accumulation of the displaced strand A1 containing the 5′ terminus of the nicking site.

Besides annealing a target cDNA to a template nucleic acid molecule to provide an initial nucleic acid molecule N1 , various other methods may be used. For example, in certain embodiments, the target cDNA itself comprises a nicking agent recognition sequence and thus may function as a N1 molecule. Alternatively, a N1 molecule may be prepared using various primer pairs. Detailed descriptions for various methods for preparing initial nucleic acids are provided below in a separate section.

2. mRNA or cDNA Populations and Target mRNA or cDNA Molecules

mRNAs of the present invention may be isolated from any biological samples that may contain an mRNA molecule of interest and may be further used to prepare cDNAs. In particular, the biological sample can be any cell, organ, tissue, biopsy material, etc. Of interest are samples derived from mammals (especially human beings), plants, bacteria and lower eukaryotic cells such as yeasts, fungal cells. Exemplary biological samples include, but are not limited to, a cancer biopsy, neurodegenerative plaque, cerebral zone biopsy displaying neurodegenerative signs, a skin sample, a blood cell sample, a colorectal biopsy, etc. Exemplary cells include muscular cells, hepatic cells, fibroblasts, nervous cells, epidermal and dermal cells, blood cells such as B-, T-lymphocytes, mastocytes, monocytes, granulocytes and macrophages. In addition, because of the high sensitivity, the present methods for gene expression analysis may be used to analyze mRNA isolated from a single cell.

In certain embodiments, cDNA populations from two different biological samples are compared to identify genes that are differentially expressed. For such a comparison, one sample may be from a subject that is suspected of having, or is at risk for having, a genetic disease or a pathogen infection while the other sample may be a healthy, control subject. Alternatively, these two samples may be from a same biological source but at different developmental stages. In certain embodiments, one sample may be from a subject that possesses a desirable trait (e.g., disease resistance), while the other may be from a subject that does not have the same trait. In other embodiments, one sample is from a subject that has been treated with a chemical (e.g., a drug or a toxic material) while the other is from an untreated, control subject.

The methods for isolating mRNA and cDNA synthesis are well known in the art (see, e.g., Sambrook et al., supra; Chomczynski et al., Anal. Biochem. 162:156, 1987). Such methods generally comprise cell, tissue or sample lysis and RNA recovery by means of extraction procedures. These procedures can be done in particular by treatment with chaotropic agents such as guanidinium thiocyanate followed by RNA extraction with solvents such as phenol and chloroform. They may be readily implemented by using commercially available kits such as US73750 kit (Amersham) for total RNA isolation. mRNA molecules may be purified from total cellular RNA using oligo(dT) primers that bind the poly(A) tails of the mRNA molecules (see, Jacobson, Metho. Enzymol. 152: 254,1987, incorporated herein by reference). In this regard, the preparation of mRNA can be carried out using commercially available kits such as US72700 kit (Amersham). Alternatively, random primers (i.e., primers with random sequences) may be used for purifying mRNA from total cellular RNA (see, Singh et al., Cell 52: 415, 1988; Vinson et al., Genes Dev. 2: 801, 1988). Either the oligo(dT) primers or the random primers may be immobilized to facilitate the purification of mRNAs. In certain other embodiments, mRNA may be directly isolated from biological samples without first isolating total RNA.

The isolated/purified mRNAs may be then used as templates for synthesizing first strand cDNAs by reverse transcription according to conventional molecular biology techniques (see, e.g., Sambrook et al., supra). Reverse transcription is generally carried out using a reverse transcriptase and a primer.

Many reverse transcriptases have been described in the literature and are commercially available (e.g., 1483188 kit, Boehringer). Exemplary reverse transcriptases include, but are not limited to, those derived from avian virus AMV (Avian Myeloblastosis Virus), from murine leukemia virus MMLV (Moloney Murine Leukemia Virus), from Yhermus flavus and Thermus thermophilus HB-8 (Promega, catalog number M1941 and M2101). The operating conditions that apply to these enzymes are well known to those of ordinary skill in the art.

The primers used for reverse transcription may be of various types. It may be a random oligonucleotide comprising 4 to 10 nucleotides, preferably a hexanucleotide. Use of this type of random primer has been described in the literature and allows random initiation of reverse transcription at different sites within the RNA molecules. Alternatively, a poly(dT) primer comprising 4 to 20-mers, preferably 15 mers may be used. In certain embodiments, the primer used in isolating mRNA is also used in cDNA synthesis.

Second strand cDNA may be synthesized using an RNase H and a DNA polymerase. Alternatively, it may be synthesized by first ligating an adaptor sequence to a first strand cDNA molecule and extending a primer complementary to the adaptor sequence using the first strand cDNA as a template.

The synthesized cDNAs may be in solution or linked to a solid support, for example, via an immobilized primer for isolating mRNA and synthesizing cDNAs (such as poly(dT)n immobilized via its 5′ terminus).

Any gene whose expression is of interest may be analyzed by the present invention. In certain embodiments, the gene is associated with a disease or a disorder, particularly a human disease or disorder. In other embodiments, the gene is associated with a desirable trait of the organism from which it originates. In yet other preferred embodiments, the gene is involved in the development of the subject from which it is isolated. In some embodiments, the gene participates the responses of the organism from which it is isolated to an external stimulus (e.g., light, drug, and stress treatment).

3. Various Embodiments for Preparing Initial Nucleic Acids (N1 )

The initial nucleic acid molecules useful for gene expression analysis may be provided by various approaches. For instance, N1 may be obtained by annealing of a target cDNA or a portion thereof to a T1 molecule. Alternatively, N1 may be directly a target cDNA itself or directly derived from a target cDNA where the target cDNA is double-stranded and comprises a nicking agent recognition sequence. N1 may also be prepared using various oligonucleotide primer pairs. These and other means for providing N1 molecules are described below.

a. By Annealing

In certain embodiments of the present invention, N1 is provided by annealing a target cDNA molecule with a T1 molecule. If the target cDNA is single-stranded, it may be directly used to anneal to a T1 molecule that is at least substantially complementary to the 3′ portion of the target cDNA. Alternatively, the single-stranded target cDNA may be cleaved to produce shorter fragments, where one or more of these fragments may be used to anneal to a T1 molecule. If the target cDNA is double-stranded, it may be denatured and directly used to anneal to a T1 molecule. Alternatively, it may be first cleaved to obtain shorter double-stranded fragments, and the shorter fragments are then denatured of which one may anneal to a T1 molecule.

As discussed above, a T1 molecule must be at least substantially complementary to a single-stranded target cDNA or one strand of a double-stranded target cDNA. In addition, the number of T1 molecules in an amplification reaction mixture is preferably greater than that of the target cDNA so that it is not a limiting factor in gene expression analyses.

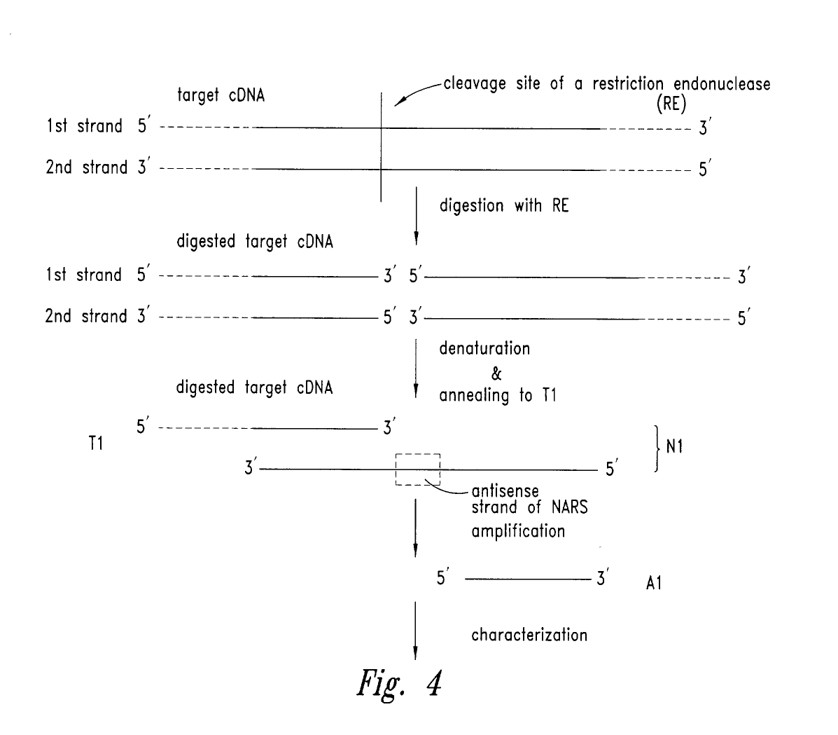

An example of this type of methods for providing N1 molecules is shown in FIG. 4. In this figure, a cDNA population that may contain a double-stranded target cDNA is digested with a restriction endonuclease that recognizes a sequence within the target cDNA. The digestion products may be denatured and one strand of a digestion product of the target cDNA, if the target cDNA is present in the cDNA population, may anneal to a T1 molecule that is at least substantially complementary to the 3′ portion of the strand of the digestion product.

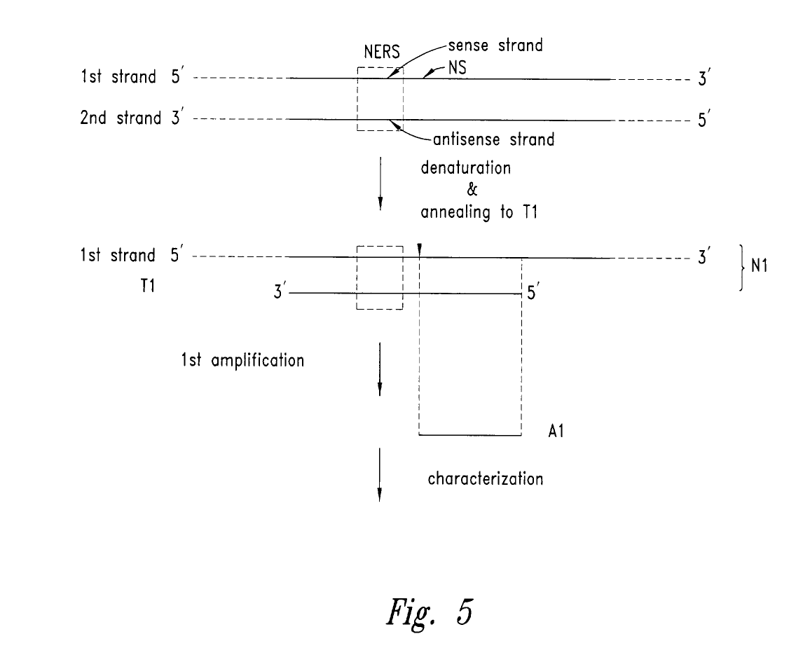

Another example of this type of methods for providing N1 molecules is shown in FIG. 5. The target cDNA (or a fragment thereof) itself contains a nicking agent recognition sequence. The target cDNA is denatured and one strand of the target cDNA anneals to a T1 molecule. The T1 molecule is a portion of the other strand of the target cDNA that comprises a sequence of the antisense strand of the nicking agent recognition sequence. The annealing of one strand of the target cDNA to the T1 molecule provides the initial nucleic acid molecule N1 for amplification reactions.

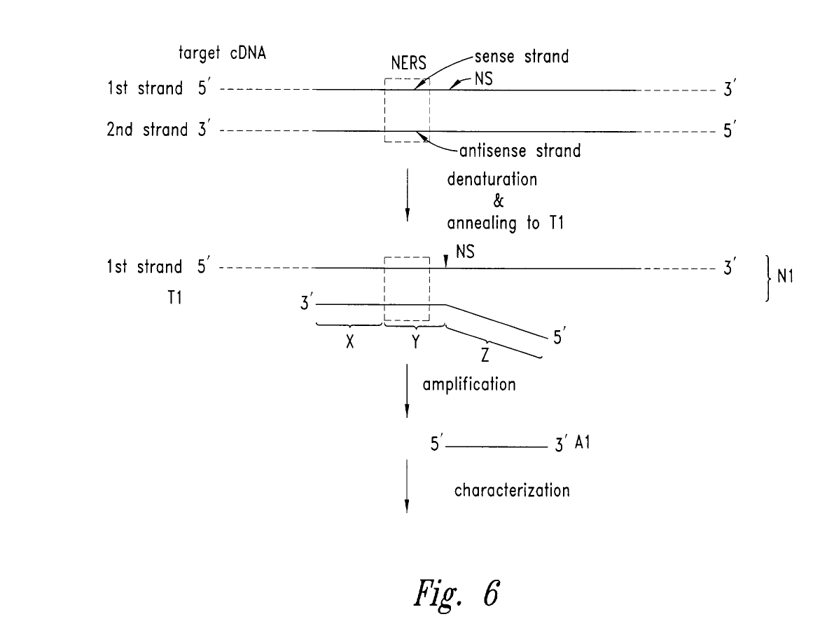

In related embodiments where a target cDNA comprises a nicking agent recognition sequence, a T1 molecule may be designed to be at least substantially complementary to the strand of the target cDNA (i.e., the first strand of the target cDNA) that comprises the sequence of the sense strand of the nicking agent recognition sequence at the 3′ portion of the T1 molecule (i.e., Regions X and Y), but not at the 5′ portion of the T1 molecule (i.e., Region Z) (FIG. 6). The 3′ portion of T1 includes the sequence of the antisense strand of the NARS so that the initial nucleic acid formed by annealing T1 to the above strand of the target cDNA comprises a double-stranded NARS. In the presence of a NA that recognizes the NARS, the N1 molecule is nicked. The 3′ terminus at the nicking site is then extended using a region 5′ to the sequence of the antisense strand of the NARS in the T1 molecule as the template. The resulting amplification product is a single-stranded nucleic acid molecule that is complementary to a region of T1 located 5′ to the sequence of the antisense strand of the NARS (i.e., Region Z1) rather than a portion of the target cDNA.

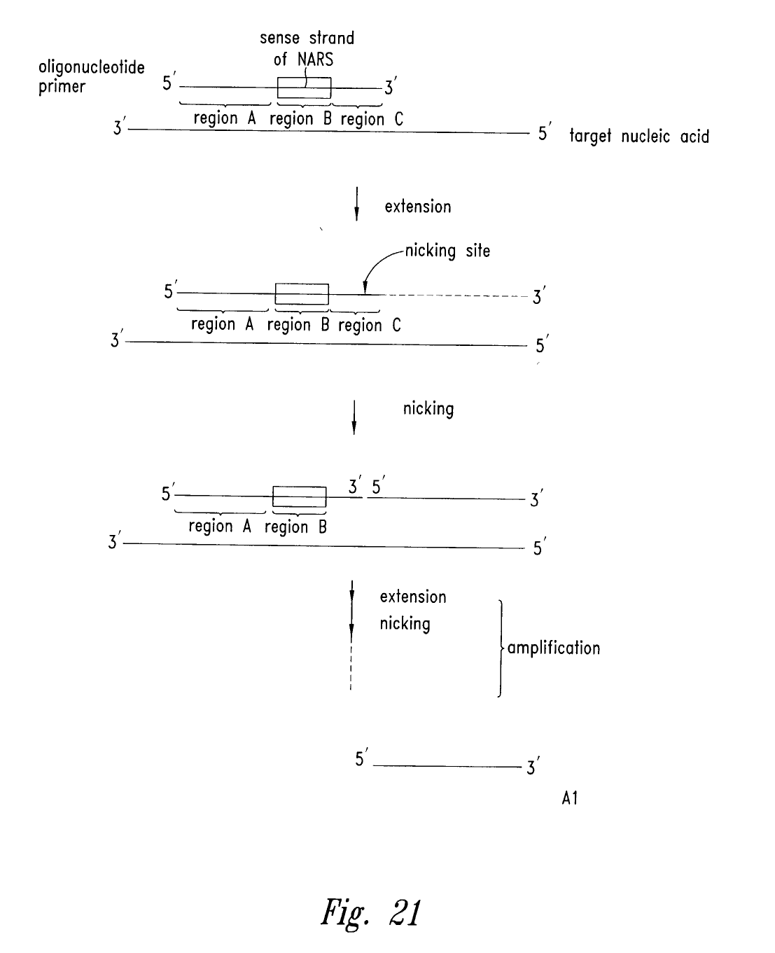

Another example of this type of methods for providing N1 molecules is shown in FIG. 21. In this example, a NARS recognizable by a nicking agent that nicks outside its NARS is used as an exemplary nicking agent. An oligonucleotide primer (i.e.,a T1 molecule) is used to amplify a single-stranded nucleic acid molecule using a portion of a single-stranded target nucleic acid (mRNA, a first strand cDNA, or one strand of a double-stranded cDNA) as a template. The primer comprises, from 5′ to 3′, three regions: Region A, Region B and Region C. Region B consists of a sequence of the sense strand of a double-stranded nicking agent recognition sequence, where Region A and Region C are regions that are located directly 5′ and 3′ to Region B, respectively. The oligonucleotide primer is at least substantially complementary to the target nucleic acid so that under conditions that allow for the amplification of a single-stranded nucleic acid, the oligonucleotide primer is able to anneal to the target and extends from its 3′ terminus in the presence of a DNA polymerase. The resulting extension product may be nicked in the presence of a nicking agent that recognizes the double-stranded nicking agent recognition sequence even though there may be one or more nucleotides in Region B of the oligonucleotide primer that do not form conventional base pairs with nucleotides in the target nucleic acid. A “conventional base pair” is a base pair formed according to the standard Watson-Crick model (e.g., G:C, A:T, and A:U) between a nucleotide of one strand of a fully or partially double-stranded nucleic acid and another nucleotide on the other strand of the nucleic acid. The nicked product that contains the 5′ terminus may readily dissociate from the target nucleic acid if it is relatively short (e.g., no longer than 18 nucleotides) or be displaced by the extension of the nicked product that contains the 3′ terminus at the nicking site. If the nicking agent nicks outside its recognition sequence, the extension product retains Region B of the oligonucleotide primer (i.e., the sequence of the sense strand of the nicking agent recognition sequence) and may thus re-nicked by the nicking agent. The above nicking-extension cycle may be repeated multiple times, resulting in the amplification of a single-stranded nucleic acid molecule that contains the 5′ terminus at the nicking site.

In embodiments where there are one or more mismatches between Region B and its corresponding region in the target nucleic acid, the nicking activity of a nicking agent that recognizes Region B decreases with the increase in the number of the mismatches between Region B and its corresponding region in the target. For example, N.BstNB I is about half as active in nicking a duplex that comprises a sequence of the sense strand of its double-stranded recognition sequence but has one mismatch between the sense strand of its recognition sequence and its corresponding region in the opposite strand of the duplex as in nicking a duplex that comprises a double-stranded recognition sequence. The nicking activity of N.BstNB I decreases to about 10% to 20% of its maximum level when it nicks a duplex that comprises a sequence of the sense strand of its double-stranded recognition sequence but does not have any nucleotides in the other strand that form conventional base pairs with any of the nucleotides in the sense strand of the recognition sequence.

In certain embodiments, a nicking agent that nicks within its recognition sequence may also be used where the nucleotide(s) in Region B that does not form a conventional base pair with a nucleotide in the target is located 5′ to the nicking site within Region B. After the duplex formed between the oligonucleotide primer and the target is nicked by the nicking agent within Region B, the 3′ terminus at the nicking site may be extended to regerate Region B. Such regeneration allows for the repetition of the nicking-extension cycles. In addition, the mismatch(es) between Region B and the corresponding region in the target must not affect the extension from the 3′ terminus at the nicking site. Generally, the more distance between the nicking site and the nucleotide(s) in Region B that does not form a conventional base pair, the less adverse effect the mismatch(es) has on the extension.

Region A facilitates or enables the annealing of the oligonucleotide primer to the target nucleic acid. In addition, it facilitates or enables the nicked product that contains the 3′ terminus at the nicking site to remain annealing to the target and to extend from the 3′ terminus in the presence of a DNA polymerase. In certain embodiments, Region A is at most 100, 75, 50, 25, 20, 15, 14, 13, 12, 11, 10, 9, 8, 7, 6, 5, 4, 3, or 2 nucleotides in length. In some embodiments, there may be one or more nucleotides that do not form conventional base pairs in Region A with the nucleotides in the target nucleic acid.

An oligonucleotide primer may or may not have a Region C. If Region C is present, in certain embodiment, it may be at most 100, 75, 50, 25, 20, 15, 14, 13, 12, 11, 10, 9, 8, 7, 6, 5, 4, 3, 2 or 1 nucleotide(s) in length. There may be mismatch(es) between Region C and its corresponding region in a target nucleic acid. However, the presence of the mismatch(es) need still allow for the nicking of the duplex formed between the oligonucleotide primer and the target or the nicking of the extension product of the duplex. In addition, the presence of the mismatch(es) need still allow for the extension of the nicked product that contains the 3′ terminus at the nicking site to extend from that terminus in the presence of a DNA polymerase. If Region C comprises a nicking site nickable by a nicking agent that recognizes Region B, generally, the nucleotides between the 5′ terminus of Region C and the nicking site forms conventional base pairs with nucleotides in the target.

The present invention is useful to detect the presence of a target nucleic acid (i.e. a target mRNA or cDNA) in a sample. If the target nucleic acid is present in a sample, it will anneal with an oligonucleotide primer (i.e. a T1 molecule) that is at least substantially complementary to the target and initiates the amplification of a single-stranded nucleic acid (i.e., an A1 molecule) using a portion of the target as a template. However, if the target nucleic acid is absent, the oligonucleotide primer will not be able to anneal with the target, and no single-stranded nucleic acid molecule using a portion of the target as a template will be amplified. Thus, by determining the presence or absence of the single-stranded amplification product, one is able to determine the presence or absence of the target nucleic acid in the sample.

The target mRNA or cDNA can be any mRNA or cDNA of interest. Because the presence of a sequence of the sense strand of a double-stranded nicking agent recognition sequence in an oligonucleotide primer is sufficient for the duplex formed between the primer and the target to be nicked by a nicking agent that recognizes the double-stranded nicking agent recognition sequence, the target is not required to have an intact antisense strand of the double-stranded recognition sequence or even any of the nucleotides that form conventional base pairs with nucleotides within the sense strand of the recognition sequence. However, because the nicking activity of a nicking agent decreases with the increase in the number of the nucleotides of the sense strand of the recognition sequence that do not form conventional base pairs with the nucleotides of the opposite strand, it is preferred to design the oligonucleotide prime so that when it anneals to a portion of a target nucleic acid, the nucleotides in the sense strand of the recognition sequence in the primer forms one or more conventional base pairs with nucleotides of the target.

In certain embodiments, as described below, it may be desirable to synthesize a relatively short single-stranded nucleic acid. In such embodiments, the target nucleic acid may be first subject to enzymatic, chemical, or mechanic cleavages. Relatively short single-stranded nucleic acids include those that have at most 200, 150, 100, 75, 50, 40, 30, 25, 20, 18, 16, 14, 12, 10, 9, 8, 7, 6, 5 or 4 nucleotides. Enzymatic cleavages may be accomplished, for example, by digesting the nucleic acid molecule with a restriction endonuclease that recognizes a specific sequence within the target nucleic acid. Alternatively, enzymatic cleavages may be accomplished by nicking the target nucleic acid with a nicking agent that recognizes a specific sequence within the nucleic acid molecule. Enzymatic cleavages may also be oligonucleotide-directed cleavages according to Szybalski (U.S. Pat. No. 4,935,357). Chemical and mechanic cleavages may be accomplished by any method known in the art suitable for cleaving nucleic acid molecules such as shearing. The cleavage product, if double-stranded, may be first denatured and subsequently anneal to an oligonucleotide primer described above.

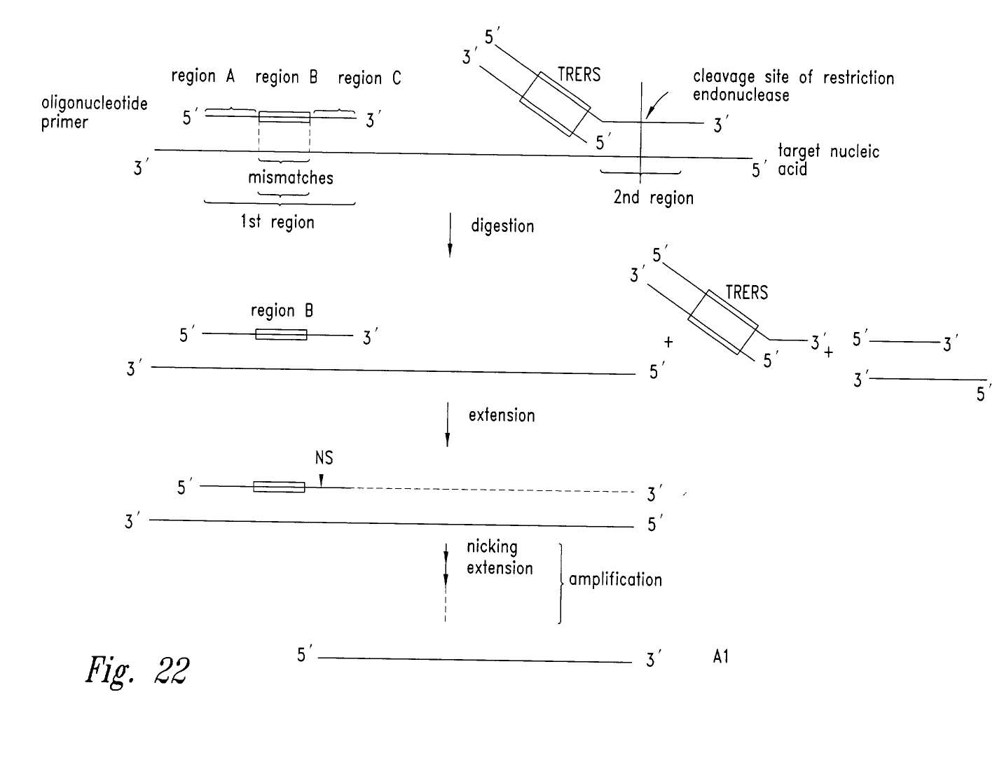

One exemplary embodiment of enzymatic cleavage of a target nucleic acid and subsequent amplification of a single-stranded nucleic acid that is complementary to a portion of the target is illustrated in FIG. 22. An oligonucleotide primer that comprises a sequence of the sense strand of a double-stranded nicking agent recognition sequence is annealed to a first region of a single-stranded target nucleic acid (i.e., mRNA, first strand of cDNA, or second strand of cDNA), whereas a partially double-stranded nucleic acid is annealed to a second region of the target nucleic acid located 5′ to the first region. The double-stranded nucleic acid molecule comprises a double-stranded recognition sequence of a type II restriction enzyme recognition sequence (TRERS) in the double-stranded portion and a 3′ overhang that is at least substantially, preferably exactly, complementary to a portion of the second region of the target nucleic acid. Because type IIs restiction endonuclease cleaves a nucleic acid outside its double-stranded recognition sequence, the partially double-stranded nucleic acid molecule may be designed to cleave within the duplex formed between the 3′ overhang of the partially double-stranded nucleic acid molecule and the second region of the target nucleic acid. Such cleavage results in a shorter fragment of the target nucleic acid to be used as a template to amplify a single-stranded nucleic acid fragment.

In certain embodiments, the double-stranded nicking agent recognition sequence of which the sense strand is present in Region B of an oligonucleotide primer may be identical to the double-stranded TRERS. For instance, Region B of the oligonucleotide primer may consist of the sequence “5′-GAGTC-3′” recognizable by a nicking endonuclease N.BstNB I, while the TRERS in the partially double-stranded nucleic acid molecule may be

5′-GAGTC-3′

3′-CTCAG-5′

recognizable by type IIs restriction endonuclease PleI and MlyI. In such embodiments, there need be mismatch(es) between Region B of the oligonucleotide primer and the corresponding region in the target nucleic acid. In other words, one or more nucleotides in Region B do not form conventional base pairs with nucleotides in the target. The presence of mismatches prevents the cleavage of the duplex formed between the oligonucleotide primer and the first region of the target by a type IIs restriction endonuclease that recognizes the TRERS.

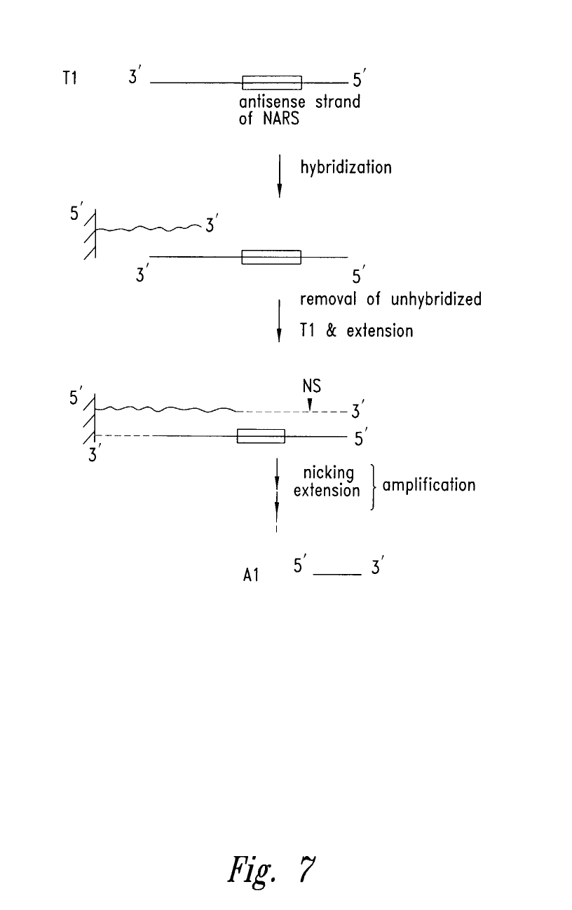

Another example of this type of methods for providing N1 molecules is shown in FIG. 7. In this example, a nicking agent recognition sequence recognizable by a nicking agent that nicks outside the recognition sequence is used as an exemplary recognition sequence. As shown in this figure, the cDNA molecules of the cDNA population are immobilized via their 5′ termini. The immobilized nucleic acid are mixed with a T1 molecule that comprises, from 3′ to 5′, a sequence that is at least substantially complementary to a target cDNA that may be present in the cDNA population, and a sequence of the antisense strand of a nicking agent recognition sequence. If the target cDNA is present in the cDNA population, the T1 molecule hybridizes to the target nucleic acid to form a N1 molecule and may be separated from unhybridized T1 molecule by washing the solid phase to which the target cDNA is attached. In the presence of a DNA polymerase and a nicking agent that recognizes the nicking agent recognition sequence, N1 is used as a template to amplify a single-stranded nucleic acid molecule A1 . However, if the target cDNA is absent in the cDNA population, T1 is unable to hybridize to any cDNA molecules in the population and thus is washed off from the solid support. Consequently, no N1 can be formed that attaches to the solid support, and no single-stranded nucleic acid molecule complementary to a portion of N1 can be amplified.

b. Target cDNAs Comprising Nicking Agent Recognition Sequence

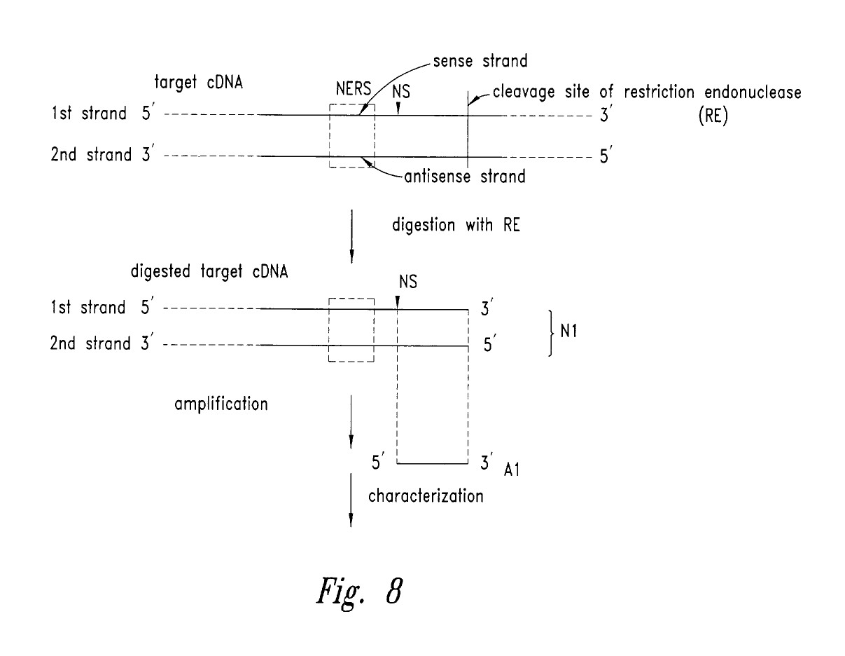

In certain embodiments, a target cDNA itself contains a double-stranded nicking agent recognition sequence and may directly function as a N1 molecule if present in a cDNA population. If the target cDNA also contains a restriction endonuclease recognition sequence, it may be first digested by a restriction endonuclease that recognizes the restriction endonuclease recognition sequence. The digestion product that contains the nicking agent recognition sequence may function as an initial nucleic acid molecule (N1 ). An embodiment with a nicking endonuclease recognition sequence recognizable by a nicking endonuclease that nicks outside its recognition sequence (e.g., N.BstNB I) as an exemplary nicking agent recognition sequence is illustrated in FIG. 8.

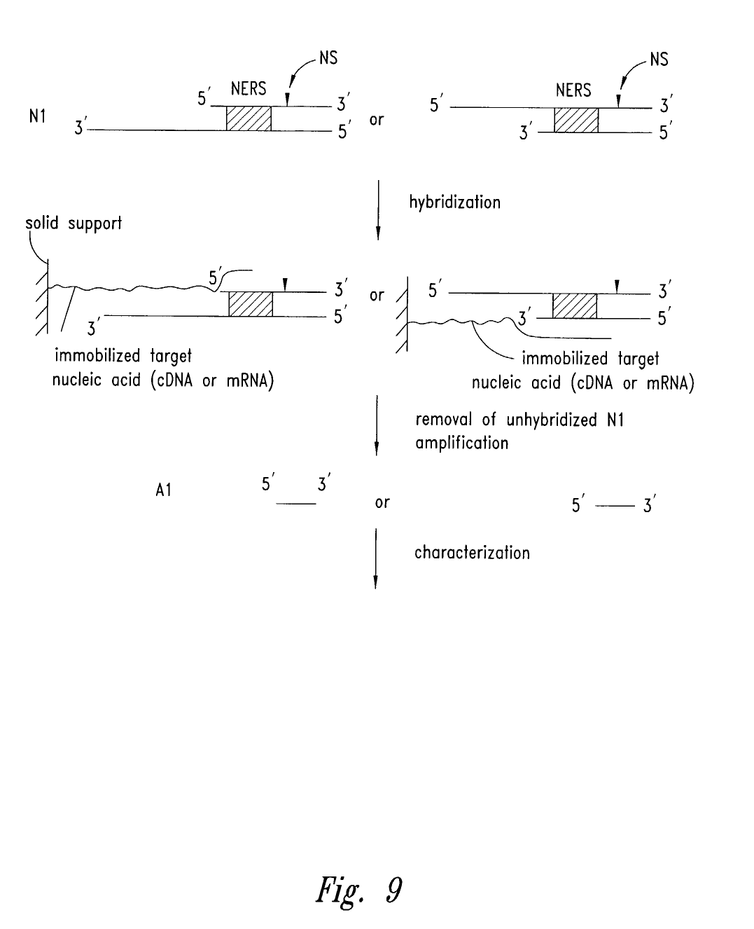

In other embodiments, an initial nucleic acid molecule N1 is a partially double-stranded nucleic acid molecule having a nicking agent recognition sequence and an overhang at least substantially complementary to a target cDNA or a target mRNA. An exemplary embodiment wherein N1 has a nicking endonuclease recognition sequence recognizable by a nicking endonuclease that nicks outside its recognition sequence as an exemplary nicking agent recognition sequence is illustrated in FIG. 9. As shown in this figure, the N1 molecule may contain a 5′ overhang in the strand that either comprises a nicking site or forms a nicking site upon extension. Alternatively, the N1 molecule may contain a 3′ overhang in the strand that neither comprises a nicking site nor forms a nicking site upon extension. The overhang of the N1 molecule must be at least substantially complementary to a target cDNA molecule (or a target mRNA) so that it can anneal to the target nucleic acid molecule. The annealing of N1 to the target cDNA (or a target mRNA) enables the isolation of a complex formed between the target cDNA and the N1 molecule (“target-N1 complex”) in those instances where the target cDNA is present in a cDNA population of interest or where the target mRNA is present in a biological sample of interest.

For instance, the cDNA molecules in a cDNA population or the mRNA molecules in a biological sample may be immobilized to a solid support as shown in FIG. 9. Such immobilization may be performed by any method known in the art, including without limitation, the use of a fixative or tissue printing. A N1 molecule having an overhang that is substantially complementary to a particular target cDNA or a target mRNA is then applied to the cDNA population or the biological sample. If the target cDNA is present in the cDNA population or the target mRNA is present in the biological sample, N1 hybridizes to the target nucleic acid via its overhang. The cDNA population or the biological sample is subsequently washed to remove any unhybridized N1 molecule. In the presence of a DNA polymerase and a nicking endonuclease that recognizes the NERS in N1 , a single-stranded nucleic acid molecule A1 is amplified. However, if the target cDNA or mRNA is absent in the cDNA population (or the biological sample), N1 is unable to hybridize to any nucleic acid molecule in the sample and thus is washed off from the sample. Thus, when the washed cDNA population (or the biological sample) is incubated with a nucleic acid amplification reaction mixture (i.e., a mixture containing all the necessary components for single-stranded nucleic acid amplification using a portion of N1 as a template, such as a NE that recognizes the NERS in the N1 molecule and a DNA polymerase), no single-stranded nucleic acid molecule that is complementary to the above portion of N1 is amplified.

Besides immobilizing a target nucleic acid molecule, a target-N1 complex may be purified by first hybridizing the N1 molecule with the target cDNA (or mRNA) molecule in a cDNA population (or a biological sample) and then isolating the complex by a functional group associated with the target nucleic acid. For instance, the cDNA molecules in the cDNA population may be labeled with a biotin molecule, and the target-N1 complex may be subsequently purified via the biotin molecule associated with the target, such as precipitating the complex with immobilized streptavidin.

c. Using Oligonucleotide Primers

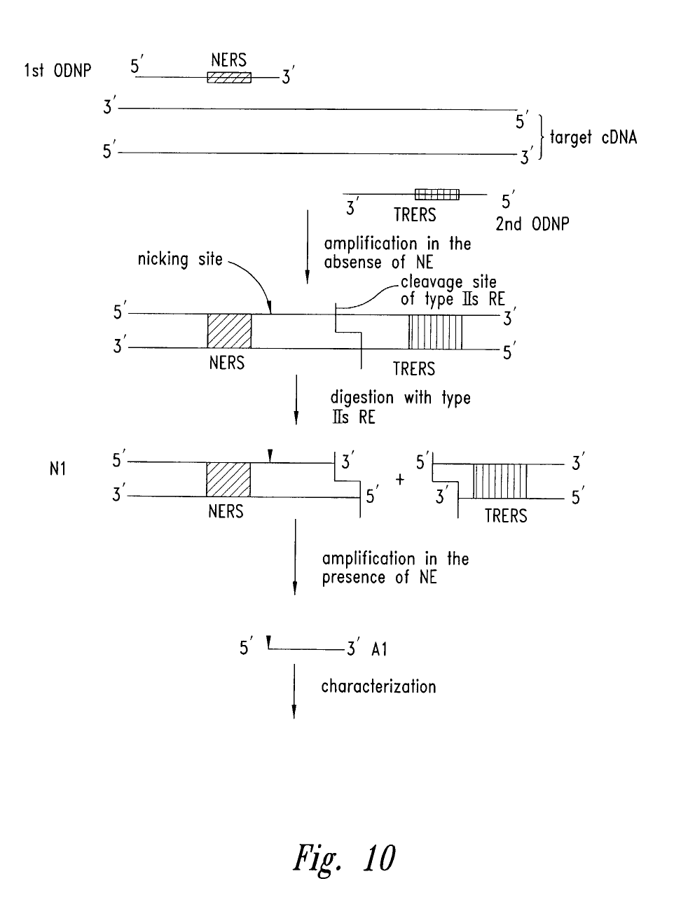

In certain embodiments of the present invention, an initial nucleic acid molecule N1 is a completely or partially double-stranded nucleic acid molecule produced using various oligonucleotide primer pairs. The methods for using ODNP pairs to prepare N1 molecules are described below in connection with FIGS. 10-12.

In one embodiment, a precursor to N1 contains a double-stranded NARS and a RERS. The NARS and RERS are incorporated into the precursor using an ODNP pair. An embodiment with a NERS recognizable by a NE that nicks outside its recognition sequence (e.g., N.BstNB I) as an exemplary NARS, and a type IIs restriction endonuclease recognition sequence (TRERS) as an exemplary RERS is illustrated in FIG. 10. As shown in this figure, a first ODNP comprises the sequence of one strand of a NERS while a second ODNP comprises the sequence of one strand of a TRERS. When these two ODNPs are used as primers to amplify a portion of a target cDNA, the resulting amplification product (i.e., a precursor to N1 ) contains both a double-stranded NERS and a double-stranded TRERS. In the presence of a type IIs restriction endonuclease that recognizes the TRERS, the amplification product is digested to produce a nucleic acid molecule N1 that comprises a double-stranded NERS.

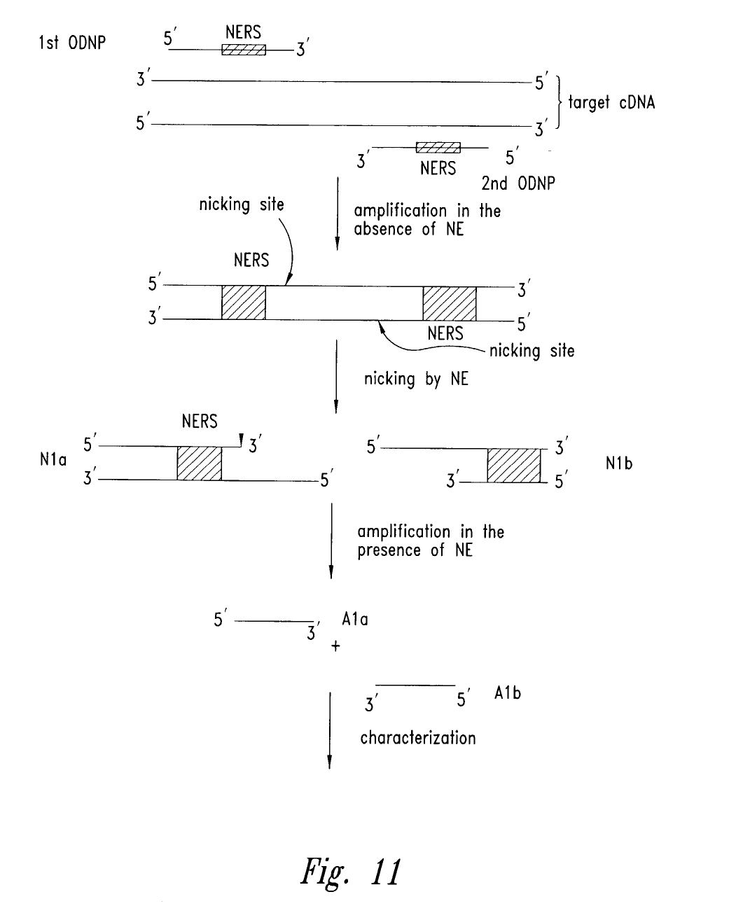

In another embodiment, a precursor to N1 contains two double-stranded NARSs. The two NARSs are incorporated into the precursor to N1 using two ODNPs. An embodiment with a NERS recognizable by a nicking endonuclease that nicks outside its recognition sequence as an exemplary NARS is illustrated in FIG. 11. As shown in this figure, both ODNPs comprise a sequence of a sense strand of a NERS. When these two ODNPs are used as primers to amplify a portion of a target cDNA, the resulting amplification product contains two NERSs. These two NERSs may or may not be identical to each other, but preferably, they are identical. In the presence of a NE or NEs that recognize the NERSs, the amplification product is nicked twice (once on each strand) to produce two nucleic acid molecules (N1a and N1b) that each comprises a double-stranded NERS.

In yet another embodiment, a precursor to N1 contains two hemimodified RERS. The two hemimodified RERSs are incorporated into the precursor by the use of two ODNPs. This embodiment is illustrated in FIG. 11. As shown in this figure, both the first and the second ODNPs comprise a sequence of one strand of a RERS. When these two ODNPs are used as primers to amplify a portion of a target cDNA in the presence of a modified deoxynucleoside triphosphate, the resulting amplification product contains two hemimodified RERSs. These two hemimodified RERS may or may not be identical to each other. In the presence of a RE or REs that recognize the hemimodified RERS, the above amplification product is nicked to produce two partially double-stranded nucleic acid molecule (N1a and N1b) that each comprises a sequence of at least one strand of the hemimodified RERS.

4. Nicking Agents

Any enzyme that recognizes a specific nucleotide sequence and cleaves only one strand of a nucleic acid that comprises the sequence may be used as a nicking agent in the present invention. Such an enzyme can be a NE that recognizes a specific sequence that consists of native nucleotides or a RE that recognizes a hemimodified recognition sequence.

A nicking endonuclease may or may not have a nicking site that overlaps with its recognition sequence. An exemplary NE that nicks outside its recognition sequence is N.BstNB I, which recognizes a unique nucleic acid sequence composed of 5′-GAGTC-3′, but nicks four nucleotides beyond the 3′ terminus of the recognition sequence. The recognition sequence and the nicking site of N.BstNB I are shown below with “▾” to indicate the cleavage site where the letter N denotes any nucleotide:

▾
5′-GAGTCNNNNN-3′
3′-CTCAGNNNNN-5′

N.BstNB I may be prepared and isolated as described in U.S. Pat. No. 6,191,267. Buffers and conditions for using this nicking endonuclease are also described in the '267 patent. An additional exemplary NE that nicks outside its recognition sequence is N.Alwl, which recognizes the following double-stranded recognition sequence:

▾
5′-GGATCNNNNN-3′
3′-CCTAGNNNNN-5′

The nicking site of N.Alwl is also indicated by the symbol “▾”. Both NEs are available from New England Biolabs (NEB). N.Alwl may also be prepared by mutating a type IIs RE Alwl as described in Xu et al. (Proc. Natl. Acad. Sci. USA 98:12990-5, 2001).

Exemplary NEs that nick within their NERSs include N.BbvCl-a and N.BbvCl-b. The recognition sequences for the two NEs and the NSs (indicated by the symbol “▾”) are shown as follows:

| | |
|---|---|
| N.BbvCI-a | |
| | ▾ |
| | 5′-CCTCAGC-3′ |
| | 3′-GGAGTCG-5′ |
| N.BbvCI-b | |
| | ▾ |
| | 5′-GCTGAGG-3′ |
| | 3′-CGACTCC-5′ |

Both NEs are available from NEB.

Additional exemplary nicking endonucleases include, without limitation, N.BstSE I (Abdurashitov et al., Mol. Biol. (Mosk) 30:1261-7,1996), an engineered EcoR V (Stahl et al., Proc. Natl. Acad. Sci. USA 93: 6175-80,1996), an engineered Fok I (Kim et al., Gene 203: 43-49, 1997), endonuclease V from Thermotoga maritima (Huang et al., Biochem. 40: 8738-48, 2001), Cvi Nickases (e.g., CviNY2A, CviNYSI, Megabase Research Products, Lincoln, Nebr. (Zhang et al., Virology 240: 366-75,1998; Nelson et al., Biol. Chem. 379: 423-8, 1998; Xia et al., Nucleic Acids Res. 16: 9477-87, 1988), and an engineered Mly I (i.e., N.Mly I) (Besnier and Kong, EMBO Reports 2: 782-6, 2001). Additional NEs may be obtained by engineering other restriction endonuclease, especially type IIs restriction endonucleases, using methods similar to those for engineering EcoR V, Alwl, Fok I and/or Mly I.

A RE useful as a nicking agent can be any RE that nicks a double-stranded nucleic acid at its hemimodified recognition sequences. Exemplary REs that nick their double-stranded hemimodified recognition sequences include, but are not limited to Ava I, BsI I, BsmA I, BsoB I, Bsr I, BstN I, BstO I, Fnu4H I, Hinc II, Hind II and Nci I. Additional REs that nick a hemimodified recognition sequence may be screened by the strand protection assays described in U.S. Pat. No. 5,631,147.

In certain embodiments, a nicking agent may recognize a nucleotide sequence in a DNA-RNA duplex and nicks in one strand of the duplex. In certain other embodiments, a nicking agent may recognize a nucleotide sequence in a double-stranded RNA and nicks in one strand of the RNA.

Certain nicking agents require only the presence of the sense strand of a double-stranded recognition sequence in an at least partially double-stranded substrate nucleic acid for their nicking activities. For instance, N.BstNB I is active in nicking a substrate nucleic acid that comprises, in one strand, the sequence of the sense strand of its recognition sequence “5′-GAGTC-3′” of which one or more nucleotides do not form conventional base pairs (e.g., G:C, A:T, or A:U) with nucleotides in the other strand of the substrate nucleic acid.

5. DNA Polymerases

The DNA polymerase useful in the present invention may be any DNA polymerase that is 5′→3′ exonuclease deficient but has a strand displacement activity. Such DNA polymerases include, but are not limited to, exo⁻ Deep Vent, exo−Bst, exo⁻ Pfu, and exo⁻ Bca. Additional DNA polymerase useful in the present invention may be screened for or created by the methods described in U.S. Pat. No. 5,631,147, incorporated herein by reference in its entirety. The strand displacement activity may be further enhanced by the presence of a strand displacement facilitator as described below.

Alternatively, in certain embodiments, a DNA polymerase that does not have a strand displacement activity may be used. Such DNA polymerases include, but are not limited to, exo−Vent, Taq, the Klenow fragment of DNA polymerase I, T5 DNA polymerase, and Phi29 DNA polymerase. In certain embodiments, the use of these DNA polymerases requires the presence of a strand displacement facilitator. A “strand displacement facilitator” is any compound or composition that facilitates strand displacement during nucleic acid extensions from a 3′ terminus at a nicking site catalyzed by a DNA polymerase. Exemplary strand displacement facilitators useful in the present invention include, but are not limited to, BMRF1 polymerase accessory subunit (Tsurumi et al., J. Virology 67: 7648-53, 1993), adenovirus DNA-binding protein (Zijderveld and van der Vliet, J. Virology 68: 1158-64, 1994), herpes simplex viral protein ICP8 (Boehmer and Lehman, J. Virology 67: 711-5, 1993; Skaliter and Lehman, Proc. Natl. Acad. Sci. USA 91: 10665-9, 1994), single-stranded DNA binding protein (Rigler and Romano, J. Biol. Chem. 270: 8910-9, 1995), phage T4 gene 32 protein (Villemain and Giedroc, Biochemistry 35: 14395-4404, 1996), calf thymus helicase (Siegel et al., J. Biol. Chem. 267:13629-35, 1992) and trehalose. In one embodiment, trehalose is present in the amplification reaction mixture.

Additional exemplary DNA polymerases useful in the present invention include, but are not limited to, phage M2 DNA polymerase (Matsumoto et al., Gene 84: 247,1989), phage PhiPRD1 DNA polymerase (Jung et al., Proc. Natl. Acad. Sci. USA 84: 8287, 1987), T5 DNA polymerase (Chatterjee et al., Gene 97:13-19, 1991), Sequenase (U.S. Biochemicals), PRD1 DNA polymerase (Zhu and Ito, Biochim. Biophys. Acta. 1219: 267-76, 1994), 9°Nₘ™ DNA polymerase (New England Biolabs) (Southworth et al., Proc. Natl. Acad. Sci. 93: 5281-5, 1996; Rodriquez et al., J. Mol. Biol. 302: 447-62, 2000), and T4 DNA polymerase holoenzyme (Kaboord and Benkovic, Curr. Biol. 5:149-57, 1995).

Alternatively, a DNA polymerase that has a 5′→3′ exonuclease activity may be used. For instance, such a DNA polymerase may be useful for amplifying short nucleic acid fragments that automatically dissociate from the template nucleic acid after nicking.

In certain embodiments where a nicking agent nicks in the DNA strand of a RNA-DNA duplex, a RNA-dependent DNA polymerase may be used. In other embodiments where a nicking agent nicks in the RNA strand of a RNA-DNA duplex, a DNA-dependent DNA polymerase that extends from a DNA primer, such as Avian Myeloblastosis virus reverse transcriptase (Promega) may be used. In both instances, a target mRNA need not be reverse transcribed into cDNA and may be directly mixed with a template nucleic acid molecule that is at least substantially complementary to the target mRNA.

6. A1 Molecules

As described above, an A1 molecule is amplified using a portion of N1 as a template. In certain embodiments, A1 may be relatively short and has at most 25, 20, 17, 15, 10, or 8 nucleotides. Such short length may be accomplished by appropriately designing T1 molecules or ODNPs used in making N1 molecules. For instance, for the embodiments shown in FIGS. 4-7, T1 may be designed to have a short region 5′ to the sequence of the antisense strand of a NARS. For the embodiment shown in FIG. 9, the partially double-stranded N1 molecule may be designed to have a short region located 5′ to the position corresponding to the nicking site that is nickable by a nicking agent that recognizes the recognition sequence in the N1 . For the embodiments shown in FIGS. 10-12, the ODNP pair may be designed to be close to each other when the primers anneal to the target nucleic acid. The short length of an A1 molecule may be advantageous because it increases amplification efficiencies and rates. In addition, it allows the use of a DNA polymerase that does not have a stand displacement activity. It also facilitates the detection of A1 molecules in which A1 is used as an initial amplification primer via certain technologies such as mass spectrometric analysis.

7. Reaction Conditions

The present invention amplified a single-stranded nucleic acid molecule in the presence of a nicking agent and a DNA polymerase. In such an amplification reaction, a DNA polymerase may be mixed with nucleic acid molecules (e.g., template nucleic acid molecules) before, after, or at the same time as, a NA is mixed with the template nucleic acid. Preferably, the nicking-extension reaction buffer is optimized to be suitable for both the NA and the DNA polymerase. For instance, if N.BstNB I is the NA and exo⁻ Vent is the DNA polymerase, the nicking-extension buffer can be 0.5×N.BstNB I buffer and 1×DNA polymerase Buffer. Exemplary 1×N.BstNB I buffer may be 10 mM Tris-HCl, 10 mM MgCl₂, 150 mM KCl, and 1 mM dithiothreitol (pH 7.5 at 25° C.). Exemplary 1×DNA polymerase buffer may be 10 mM KCl, 20 mM Tris-HCl (pH 8.8 at 25° C.), 10 mM (NH₄)₂SO₄, 2 mM MgSO₄, and 0.1% Triton x-100. One of ordinary skill in the art is readily able to find a reaction buffer for a NA and a DNA polymerase.

In addition, in certain embodiments where a DNA polymerase is dissociative (i.e., the DNA polymerase is relatively easy to dissociate from a template nucleic acid, such as Vent DNA polymerase), the ratio of a NA to a DNA polymerase in a reaction mixture may also be optimized for maximum amplification of full-length nucleic acid molecules. As used herein, a “full-length” nucleic acid molecule refers to an amplified nucleic acid molecule that contains the sequence complementary to the 5′ terminal sequence of its template. In other words, a full-length nucleic acid molecule is an amplification product of a complete gene extension reaction. In a reaction mixture where the amount of a NA is excessive with respect to that of a DNA polymerase, partial amplification products may be produced. The production of partial amplification products may be due to excessive nicking of partially amplified nucleic acid molecules by the NA and subsequent dissociation of these molecules from their templates. Such dissociation prevents the partially amplified nucleic acid molecules from being further extended.

Because different NAs or different DNA polymerases may have different nicking or primer extension activities, the ratio of a particular NA to a specific dissociative DNA polymerase that is optimal to maximum amplification of full-length nucleic acids will vary depending on the identities of the specific NA and DNA polymerase. However, for a given combination of a particular NA and a specific DNA polymerase, the ratio may be optimized by carrying out exponential nucleic acid amplification reactions in reaction mixtures having different NA to DNA polymerase ratios and characterizing amplification products thereof using techniques known in the art (e.g., by liquid chromatography or mass spectrometry). The ratio that allows for maximum production of full-length nucleic acid molecules may be used in future amplification reactions.

In certain preferred embodiments, nicking and extension reactions of the present invention are performed under isothermal conditions. As used herein, “isothermally” and “isothermal conditions” refer to a set of reaction conditions where the temperature of the reaction is kept essentially constant (i.e., at the same temperature or within the same narrow temperature range wherein the difference between an upper temperature and a lower temperature is no more than 20° C.) during the course of the amplification. An advantage of the amplification method of the present invention is that there is no need to cycle the temperature between an upper temperature and a lower temperature. Both the nicking and the extension reaction will work at the same temperature or within the same narrow temperature range. If the equipment used to maintain a temperature allows the temperature of the reaction mixture to vary by a few degrees, such a fluctuation is not detrimental to the amplification reaction. Exemplary temperatures for isothermal amplification include, but are not limited to, any temperature between 50° C. to 70° C. or the temperature range between 50° C. to 70° C., 55° C. to 70° C., 60° C. to 70° C., 65° C. to 70° C., 50° C. to 55° C., 50° C. to 60° C., or 50° C. to 65° C. Many NAs and DNA polymerases are active at the above exemplary temperatures or within the above exemplary temperature ranges. For instance, both the nicking reaction using N.BstNB I (New England Biolabs) and the extension reaction using exo⁻ Bst polymerases (BioRad) may be carried out at about 55° C. Other polymerases that are active between about 50° C. and 70° C. include, but are not limited to, exo−Vent (New England Biolabs), exo⁻ Deep Vent (New England Biolabs), exo⁻ Pfu (Strategene), exo⁻ Bca (Panvera) and Sequencing Grade Taq (Promega).

When a restriction endonuclease is used as a nicking agent, a modified deoxyribonucleoside triphosphate is needed to produce a hemimodified restriction endonuclease recognition sequence. Any modified deoxyribonucleoside triphosphate that contributes to the inhibition of cleavage of one strand of a double-stranded nucleic acid comprising the modified deoxyribonucleoside triphosphate in a restriction endonuclease recognition sequence may be used. Exemplary modified deoxyribonucleoside triphosphates include, but are not limited to, 2′-deoxycytidine 5′-O-(1-thiotriphosphate) [i.e., dCTP(.alpha.S)], 2′-deoxyguanosine 5′-O-(1 -thiotriphosphate), thymidine-5′-O-(1 -thiotriphosphate), 2′-deoxycytidine 5′-(1-thiotriphosphate), 2′-deoxyuridine 5′-triphosphate, 5-methyldeoxycytidine 5′-triphosphate, and 7-deaza-2′-deoxyguanosine 5′-triphosphate.

8. Detecting and/or Characterizing Amplified Single-Stranded Nucleic Acids

The presence of a target cDNA in a cDNA population or a target mRNA in a biological sample may be detected by detecting and/or characterizing an amplification product (A1). Any methods suitable for detecting or characterizing single-stranded nucleic acid molecules may be used. For instance, the amplification reaction may be carried out in the presence of a labeled deoxynucleoside triphosphate so that the label is incorporated into the amplified nucleic acid molecules. Labels suitable for incorporating into a nucleic acid fragment, and methods for the subsequent detection of the fragment are known in the art, and exemplary labels include, but are not limited to, a radiolabel such as ³²p, ³³p, ¹²⁵I or ³⁵S, an enzyme capable of producing a colored reaction product such as alkaline phosphatase, fluorescent labels such as fluorescein isothiocyanate (FITC), biotin, avidin, digoxigenin, antigens, haptens, or fluorochromes.

Alternatively, amplified nucleic acid molecules may be detected by the use of a labeled detector oligonucleotide that is substantially, preferably completely, complementary to the amplified nucleic acid molecules. Similar to a labeled deoxynucleoside triphosphate, the detector oligonucleotide may also be labeled with a radioactive, chemiluminescent, or fluorescent tag (including those suitable for detection using fluorescence polarization or fluorescence resonance energy transfer), or the like. See, Spargo et al., Mol. Cell. Probes 7: 395-404, 1993; Hellyer et al., J. Infectious Diseases 173: 934-41,1996; Walker et al., Nucl. Acids Res. 24: 348-53, 1996; Walker et al., Clin. Chem. 42: 9-13, 1996; Spears et al., Anal. Biochem. 247: 130-7, 1997; Mehrpouyan et al., Mol. Cell. Probes 11: 337-47, 1997; and Nadeau et al., Anal. Biochem. 276:177-87, 1999.

In certain embodiments, amplified nucleic acid molecules may be further characterized. The characterization may confirm the identities of these nucleic acid molecules and thus confirm the presence of a target cDNA in a cDNA population or a target mRNA in a biological sample. Such a characterization may be performed via any known method suitable for characterizing single-stranded nucleic acid fragments. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445, incorporated herein in their entireties.

Besides detecting and/or characterizing an amplification product to detect the presence of a target cDNA in a DNA population or a target mRNA in a biological sample, the presence of the target nucleic acid may be detected by detecting completely or partially double-stranded nucleic acid molecules produced in the amplification reactions (e.g., H1 , H2 or nicking product thereof). In certain embodiments, the detection of the double-stranded nucleic acid molecule may be performed by adding to the amplification mixture a fluorescent compound that specifically binds to double-stranded nucleic acid molecules (i.e., fluorescent intercalating agent). The addition of a fluorescent intercalating agent enables real time monitoring of nucleic acid amplification. Alternatively, to maximize the production of double-stranded nucleic acid molecules (e.g., H1 and H2 ), the NE, but not the DNA polymerase, in the nicking-extension reaction mixture may be inactivated (e.g., by heat treatment). The inactivation of the NE allows all the nicked nucleic acid molecules in the reaction mixture to be extended to produce double-stranded nucleic acid molecules. Various fluorescent intercalating agents are known in the art and may be used in the present invention. Exemplary agents include, without limitation, those disclosed in U.S. Pat. Nos. 4,119,521; 5,599,932, 5,658,735; 5,734,058; 5,763,162; 5,808,077; 6,015,902; 6,255,048 and 6,280,933, those discussed in Glazer and Rye, Nature 359: 859-61,1992 and SYBR® (Molecular Probes, Eugene Wash.).

9. Gene Expression Profiling

In addition to methods for determining whether a gene is expressed in a biological sample, the present invention also provides a method for profiling the expression of multiple genes in a sample. For example, double-stranded cDNA molecules generated using mRNAs from a biological sample may be first digested with a restriction endonuclease to provide relatively short cDNA fragments. These cDNA fragments may be mixed with a nicking agent and a DNA polymerase in a reaction buffer suitable for nucleic acid amplification. The cDNA fragments that comprise a recognition sequence of the nicking agent may thus function as templates for amplifying single-stranded nucleic acids. The amplified single-stranded nucleic acids may be separated and/or characterized. The characterization of these amplified nucleic acids may indicate the presence or absence of one or more cDNA molecules of interest. In addition, such a characterization may also function as a profile of the cDNA population derived from the biological sample, which may be compared with that of the cDNA population derived from another biological sample.

In certain embodiments, not all the amplified nucleic acids are characterized. In other words, in these embodiments, only certain amplified nucleic acids that meet a given criterion need be characterized. For instance, the amplified nucleic acid molecules may first be separated by liquid chromatography and only the fractions that contain short nucleic acid fragments are further characterized by, for example, mass chromatography. The digestion of cDNA molecules increases the amplification of relatively short fragments that are suitable for subsequent mass spectrometric analysis. However, it is not required for the cDNA molecules to be digested prior to being mixed with a nicking agent and a DNA polymerase.

10. Immobilized Nucleic Acids and Arrays of Nucleic Acids

In certain embodiments, the nucleic acids or oligonucleotides that involve in exponential nucleic acid amplification according to the present invention may be immobilized to a solid support (also referred to as a “substrate”). The nucleic acids or oligonucleotides that may be immobilized include target mRNAs or cDNAs, oligonucleotide primers useful for preparing an initial nucleic acid (described below), trigger ODNPs, and T1 molecules. In certain embodiments, such nucleic acids or oligonucleotides may be immobilized via their 5′ or 3′ termini if they are single-stranded, or via their 5′ or 3′ termini of one strand if they are double-stranded.

The methods for immobilizing a nucleic acid or an oligonucleotide are known in the art. In certain embodiments, nucleic acids or oligonucleotides (e.g., T1 molecule or ODNPs useful for preparing an N1 molecule) of the present invention are immobilized to a substrate to form an array. As used herein, an “array” refers to a collection of nucleic acids or oligonucleotides that are placed on a solid support in distinct areas. Each area is separated by some distance in which no nucleic acid or oligonucleotide is bound or deposited. In some embodiments, area sizes are 20 to 500 microns and the center to center distances of neighboring areas range from 50 to 1500 microns. The array of the present invention may contain 2-9, 10-100, 101-400, 401-1,000, or more than 1,000 distinct areas.

Generally, the nucleic acid or oligonucleotide may be immobilized to a substrate in the following two ways: (1) synthesizing the nucleic acids or the oligonucleotides directly on the substrate (often termed “in situ synthesis”), or (2) synthesizing or otherwise preparing the nucleic acid or the oligonucleotides separately and then position and bind them to the substrate (sometimes termed “post-synthetic attachment”). For in situ synthesis, the primary technology is photolithography. Briefly, the technology involves modifying the surface of a solid support with photolabile groups that protect, for example, oxygen atoms bound to the substrate through linking elements. This array of protected hydroxyl groups is illuminated through a photolithographic mask, producing reactive hydroxyl groups in the illuminated areas. A 3′-O-phosphoramidite-activated deoxynucleoside protected at the 5′-hydroxyl with the same photolabile group is then presented to the surface and coupling occurs through the hydroxyl group at illuminated areas. Following further chemical reactions, the substrate is rinsed and its surface is illuminated through a second mask to expose additional hydroxyl groups for coupling. A second 5′-protected, 3′-O-phosphoramidite-activated deoxynucleoside is present to the surface. The selective photo-de-protection and coupling cycles are repeated until the desired set of products is obtained. Detailed description of using photolithography in array fabrication may be found in the following patents or published patent applications: U.S. Pat. Nos. 5,143,854; 5,424,186; 5,856,101; 5,593,839; 5,908,926; 5,737,257; and Published PCT Patent Application Nos. WO99/40105; WO99/60156; WO00/35931.

The post-synthetic attachment approach requires a methodology for attaching pre-existing oligonucleotides to a substrate. One method uses the biotin-streptavidin interaction. Briefly, it is well known that biotin and streptavidin form a non-covalent, but very strong, interaction that may be considered equivalent in strength to a covalent bond. Alternatively, one may covalently bind pre-synthesized or pre-prepared nucleic acids or oligonucleotides to a substrate. For example, carbodiimides are commonly used in three different approaches to couple DNA to solid supports. In one approach, the support is coated with hydrazide groups that are then treated with carbodiimide and carboxy-modified oligonucleotide. Alternatively, a substrate with multiple carboxylic acid groups may be treated with an amino-modified oligonucleotide and carbodiimide. Epoxide-based chemistries are also used with amine modified oligonucleotides. Detailed descriptions of methods for attaching pre-existing oligonucleotides to a substrate may be found in the following references: U.S. Pat. Nos. 6,030,782; 5,760,130; 5,919,626; published PCT Patent Application No. WO00/40593; Stimpson et al. Proc. Natl. Acad. Sci. 92:6379-6383 (1995); Beattie et al. Clin. Chem. 41:700-706 (1995); Lamture et al. Nucleic Acids Res. 22:2121-2125 (1994); Chrisey et al. Nucleic Acids Res. 24:3031-3039 (1996); and Holmstrom et al., Anal. Biochem. 209:278-283 (1993).

The primary post-synthetic attachment technologies include ink jetting and mechanical spotting. Ink jetting involves the dispensing of nucleic acids or oligonucleotides using a dispenser derived from the ink-jet printing industry. The nucleic acid oligonucleotides are withdrawn from the source plate up into the print head and then moved to a location above the substrate. The nucleic acids or oligonucleotides are then forced through a small orifice, causing the ejection of a droplet from the print head onto the surface of the substrate. Detailed description of using ink jetting in array fabrication may be found in the following patents: U.S. Pat. Nos: 5,700,637; 6,054,270; 5,658,802; 5,958,342; 6,136,962 and 6,001,309.

Mechanical spotting involves the use of rigid pins. The pins are dipped into a nucleic acid or oligonucleotide solution, thereby transferring a small volume of the solution onto the tip of the pins. Touching the pin tips onto the substrate leaves spots, the diameters of which are determined by the surface energies of the pins, the nucleic acid or oligonucleotide solution, and the substrate. Mechanical spotting may be used to spot multiple arrays with a single nucleic acid or oligonucleotide loading. Detailed description of using mechanical spotting in array fabrication may be found in the following patents or published patent applications: U.S. Pat. Nos. 6,054,270; 6,040,193; 5,429,807; 5,807,522; 6,110,426; 6,063,339; and 6,101,946; and published PCT Patent Application Nos. WO99/36760; 99/05308; 00/01859; and 00/01798.

One of ordinary skill in the art would appreciate that besides the techniques described above, other methods may also be used in immobilizing nucleic acids or oligonucleotides to a substrate. Descriptions of such methods can be found in, but are not limited to, the following patent or published patent applications: U.S. Pat. Nos. 5,677,195; 6,030,782; 5,760,130; and 5,919,626; and published PCT Patent Application Nos. WO98/01221; WO99/41007; WO99/42813; WO99/43688; WO99/63385; WO00/40593; WO99/19341; and WO00/07022.

The substrate to which the nucleic acids or oligonucleotides of the present invention are immobilized to form an array is prepared from a suitable material. The substrate is preferably rigid and has a surface that is substantially flat. In some embodiments, the surface may have raised portions to delineate areas. Such delineation separates the amplification reaction mixtures at distinct areas from each other and allows for the amplification products at distinct areas to be analyzed or characterized individually. The suitable material includes, but is not limited to, silicon, glass, paper, ceramic, metal, metalloid, and plastics. Typical substrates are silicon wafers and borosilicate slides (e.g., microscope glass slides). An example of a particularly useful solid support is a silicon wafer that is usually used in the electronic industry in the construction of semiconductors. The wafers are highly polished and reflective on one side and can be easily coated with various linkers, such as poly(ethyleneimine) using silane chemistry. Wafers are commercially available from companies such as WaferNet, San Jose, Calif.

Depending on the contemplated application, one of ordinary skill in the art may vary the composition of immobilized molecules of the present array. For instance, the T1 or ODNP molecules of the present invention may or may not be immobilized to every distinct area of the array. Preferably, the nucleic acids or oligonucleotides in a distinct area of an array are homogeneous. More preferably, the nucleic acids or oligonucleotides in every distinct area of an array to which the nucleic acids or oligonucleotides are immobilized are homogeneous. The term “homogeneous,” as used herein, indicates that each nucleic acid or oligonucleotide molecule in a distinct area has the same sequence as another nucleic acid or oligonucleotide molecule in the same area. Alternatively, the nucleic acid or oligonucleotide in at least one of the distinct areas of an array are heterogeneous. The term “heterogeneous,” as used herein, indicates that at least one nucleic acid or oligonucleotide molecule in a distinct area has a different sequence from another nucleic acid or oligonucleotide molecule in the area. In some embodiments, molecules other than the nucleic acids or oligonucleotides described above may also be present in some or all of distinct areas of an array. For instance, a molecule useful as an internal control for the quality of an array may be attached to some or all of distinct areas of an array. Another example for such a molecule may be a nucleic acid useful as an indicator of hybridization stringency. In other embodiments, the composition of nucleic acids or oligonucleotides in every distinct area of an array is the same. Such an array may be useful in determining genetic variations in a particular gene in a selected population of organisms or in parallel diagnosis of a disease or a disorder associated with mutations in a particular gene.

Depending on the envisioned application, the immobilized nucleic acids or oligonucleotides of the present invention (e.g., the T1 or T2 molecules) may contain oligonucleotide sequences that are at least substantially complementary or identical to various target nucleic acids. Such target nucleic acids include, but are not limited to, genes associated with hereditary diseases in animals, oncogenes, genes related to disease predisposition, genomic DNAs useful for forensics and/or paternity determination, genes associated with or rendering desirable features in plants or animals, and genomic or episomic DNA of infectious organisms. An array of the present invention may contain nucleic acids or oligonucleotides that are at least substantially complementary or identical to a particular type of target nucleic acids in distinct areas. For example, an array may have a nucleic acid or an oligonucleotide that is at least substantially complementary or identical to a first gene related to disease predisposition in a first distinct area, another nucleic acid or an oligonucleotide that is at least substantially complementary or identical to a second gene also related to disease predisposition in a second distinct area, yet another nucleic acid or an oligonucleotide that is at least substantially complementary or identical to a third gene also related to disease predisposition in a third distinct area, etc. Such an array is useful to determine disease predisposition of an individual animal (including a human) or a plant. Alternatively, an array may have nucleic acids or oligonucleotides that are at least substantially complementary or identical to multiple types of target nucleic acids categorized by the functions of the targets.

In addition, an array may contain nucleic acids or oligonucleotides that are at least substantially complementary or identical to a portion of a target nucleic acid that contains various potential genetic variations. For instance, a first area of the array may contain immobilized nucleic acids or oligonucleotides that are at least substantially complementary or identical to a portion of a target gene that contains a genetic variation of one allele of the target. A second area of the array may contain immobilized nucleic acids or oligonucleotides that are at least substantially complementary or identical to a portion of target gene that contains a genetic variation of another allele of the target. The array may have additional areas that contain immobilized nucleic acids or oligonucleotides that are at least substantially complementary or identical to portions of the target gene that contains genetic variations of additional alleles of the target.

In general, for successful performance in an array environment, the immobilized nucleic acids or oligonucleotides must be stable and not dissociate during various treatment, such as hybridization, washing or incubation at the temperature at which an amplification reaction is performed. The density of the immobilized nucleic acids or oligonucleotides must be sufficient for the subsequent analysis. For an array suitable for the present methods, typically 1000 to 10¹², preferably 1000 to 10⁶, 10⁶ to 10⁹, or 10⁹ to 10¹² ODNP molecules are immobilized in at least one distinct area. However, there must be minimal non-specific binding of other nucleic acids to the substrate. The immobilization process should not interfere with the ability of immobilized nucleic acids or oligonucleotides required for exponential nucleic acid amplification.

In certain embodiments, it may be desirable to have the nucleic acids or oligonucleotides of the present invention indirectly bound to the substrate via a linker. The linker (also referred to as a “linking element”) comprises a chemical chain that serves to distance the nucleic acids or oligonucletides from the substrate. In certain embodiments, the linker may be cleavable. There are a number of ways to position a linking element. In one common approach, the substrate is coated with a polymeric layer that provides linking elements with a lot of reactive ends/sites. A common example is glass slides coated with polylysine, which are commercially available. Another example is substrates coated with poly(ethyleneimine) as described in Published PCT Application No. W099/04896 and U.S. Pat. No. 6,150,103.

For the nucleic acid molecules of the present invention that do not form an array, they may be immobilized via the methods described above that are useful in preparing an array. In addition, any methods known in the art may be used. For instance, a target mRNA of the present invention may be immobilized by the use of a fixative or tissue printing. A target cDNA may be first synthesized and then immobilized to a substrate that binds to nucleic acids or oligonucleotides, such as nitrocellulose or nylon membranes. Alternatively, a target cDNA may be synthesized directly on a substrate, such as via an oligonucleotide primer immobilized to the substrate.

C. Gene Expression Analyses Using Exponential Nucleic Acid Amplification Methods

In one aspect, the present invention exponentially amplifies a single-stranded nucleic acid molecule in the presence of a target cDNA or a target mRNA. The exponential nucleic acid amplification increases the sensitivity of detecting the amplified single-stranded nucleic acid molecule, and thus increases the sensitivity of detecting the presence of the target cDNA or mRNA.

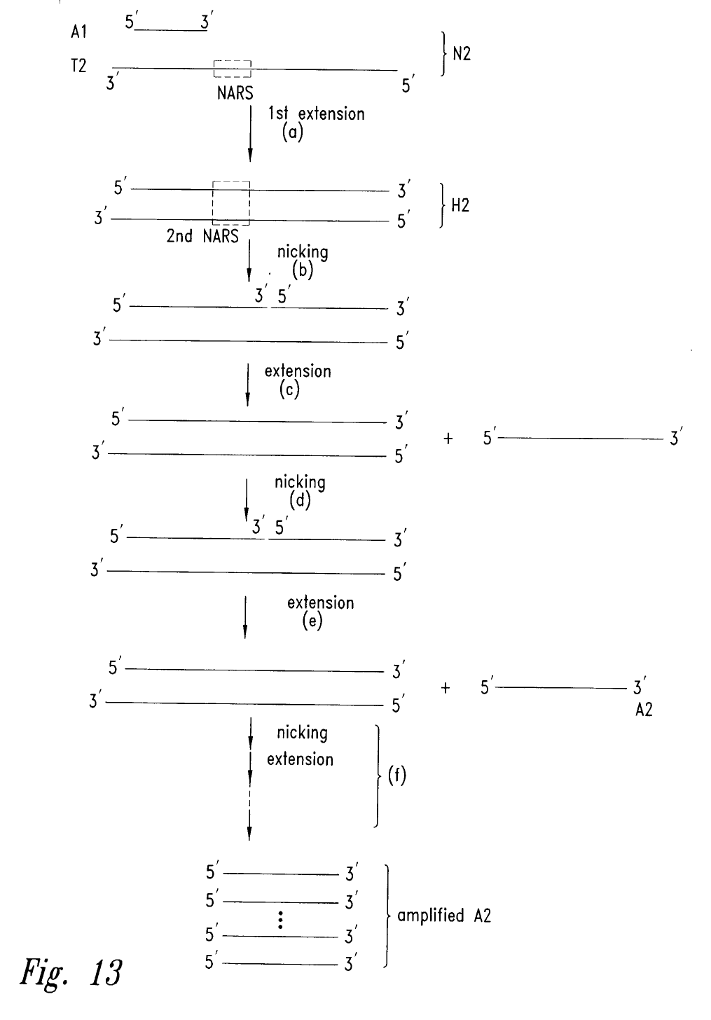

The exponential nucleic acid amplification is performed by linking the linear nucleic acid amplification reaction described above with at least another nucleic acid amplification reaction. The major steps of the second amplification reaction are illustrated in FIG. 13. In this reaction, the single-stranded nucleic acid molecule (A1) amplified in a first nucleic acid amplification reaction (FIG. 1) may be used as an initial amplification primer in the presence of a second template nucleic acid (T2) molecule. T2 comprises from 3′ to 5′: a sequence that is substantially complementary to A1 , a sequence of one strand of a nicking agent recognition sequence. When A1 anneals to T2 , the resulting partially double-stranded nucleic acid molecule is referred to as “the initial nucleic acid molecule of the second amplification reaction (N2).” In the presence of a DNA polymerase, the extension from A1 produces a hybrid (H2 ) that comprises the double-stranded nicking agent recognition sequence (step (a)). In the presence of a nicking agent that recognizes the recognition sequence, H2 is nicked, producing a 3′ terminus and a 5′ terminus at the nicking site (step (b)). If the fragment containing the 5′ terminus at the nicking site is sufficiently short (e.g., less than 18 nucleotides in length), it may dissociate from the other portion of H2 under certain conditions (e.g., at 60° C.). However, if this fragment does not readily dissociate from the other portion of H2 , it may be displaced by extension of the fragment having a 3′ terminus at the nicking site in the presence of a DNA polymerase that is 5′→3′ exonuclease deficient and has a strand displacement activity (step (c)). Strand displacement may also occur in the presence of a strand displacement facilitator. Such extension recreates a new nicking site that can be re-nicked by the nicking agent (step (d)). The fragment containing the 5′ terminus at the new NS (referred to as “A2 ”) may again readily dissociate from the other portion of H2 or be displaced by extension from the 3′ terminus at the nicking site (step (e). The nicking-extension cycles can be repeated multiple times (step (f)), resulting the exponentially accumulation/amplification of the nucleic acid fragment A2 .

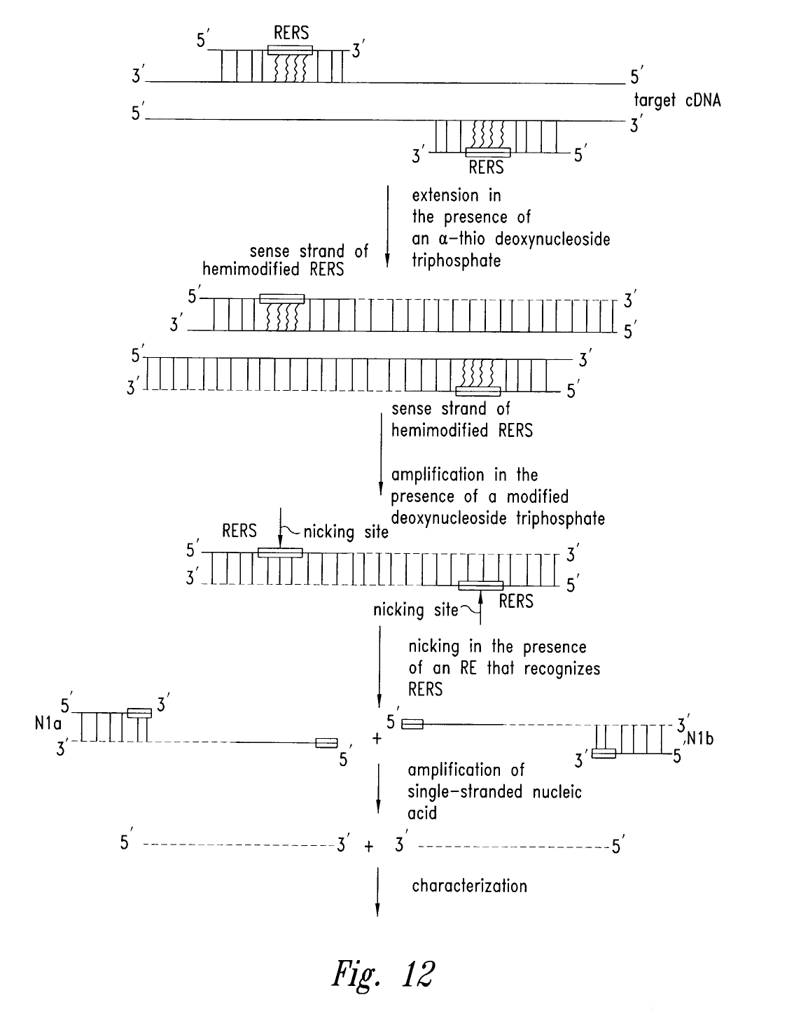

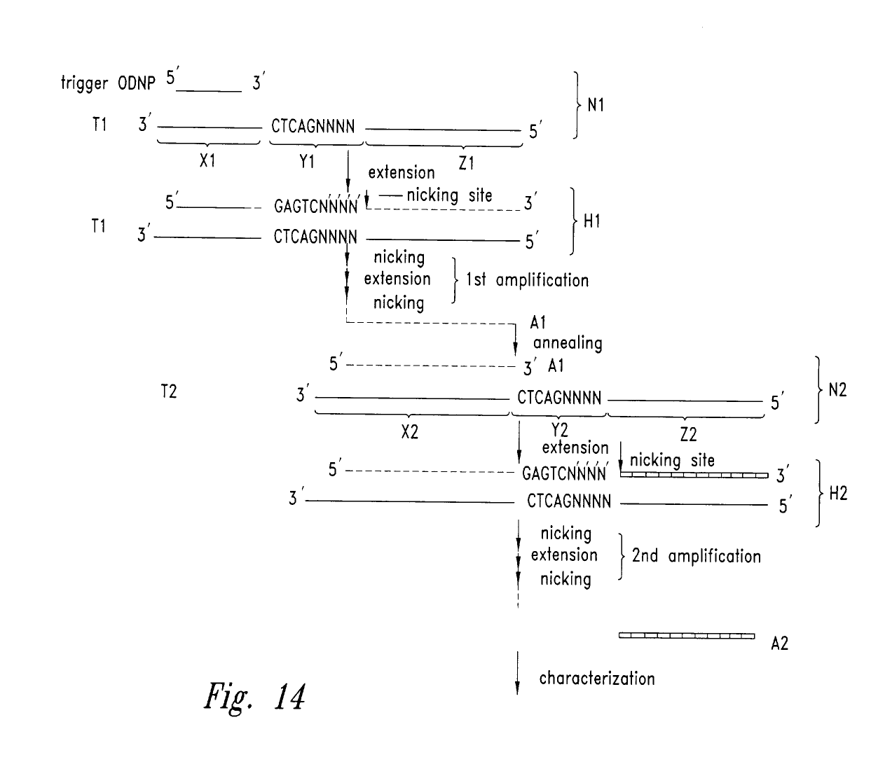

As noted above, a T2 molecule comprises a sequence of one strand of a nicking agent recognition sequence. In certain embodiments, a T2 molecule may comprise a sequence of the antisense strand of a nicking agent recognition sequence. An example of such embodiments are shown in FIG. 12 using the recognition sequence of N.BstNB I as an exemplary nicking agent recognition sequence. In FIG. 14, the amplification of A1 is the same as that in FIG. 2, where T1 comprises a sequence of the antisense strand of a nicking agent recognition sequence. A1 is then annealed to Region X2 of a second template (T2 ), which also has two additional regions: Regions Y2 and Z2, to form an initial nucleic acid molecule N2 for the second amplification reaction. Region Y2 has a similar sequence as Region Y1 (i.e., 3′-CTCAGNNNN-5′ where the Ns in Region Y2 may be identical to, or different from, those at the same positions in Region Y1), whereas Regions X2 and Z2 refer to regions immediately next to the 3′ terminus and the 5′ terminus of Region Y2, respectively. The extension of A1 using T2 as a template produces a double-stranded nucleic acid fragment (H2) or a partially double-stranded nucleic acid fragment (H2), depending on whether the 5′ terminal sequence of A1 anneals to the 3′ terminal sequence of Region X2. The resulting H2 comprises the double-stranded N.BstNB I recognition sequence, which can be nicked by N.BstNB I. The 3′ terminus at the nicking site may be extended again by the DNA polymerase, displacing the strand A2 containing the 5′ terminus at the nicking site. The nicking-extension cycle is repeated multiple times, resulting in the accumulation/amplification of the displaced strand A2 . The amplification of A2 is exponential because it is the final amplification product of two linked linear amplification reactions.

Because A2 is amplified using Region Z2 as a template, A2 may be designed to have an at least substantially identical sequence to, or a different sequence from, A1 by designing Region Z2 to have a sequence at least substantially complementary to A1 or a sequence that is not substantially complementary to A1 . In one embodiment, Region Z2 is at least substantially complementary to A1 , so that both Regions X2 and Z2 may anneal to A1 . The annealing of A1 to Z2, however, may be displaced by the extension from the 3′ terminus of A1 or 3′ terminus of a nicked product of H2 at the nicking site, and thus will not significantly affect the rate of A2 amplification. Because, in this embodiment, A2 is at least substantially identical to A1 , A2 may also anneal to Region X2 and initiate its own amplification. Such amplification may dramatically increase the rate and level of A2 amplification.

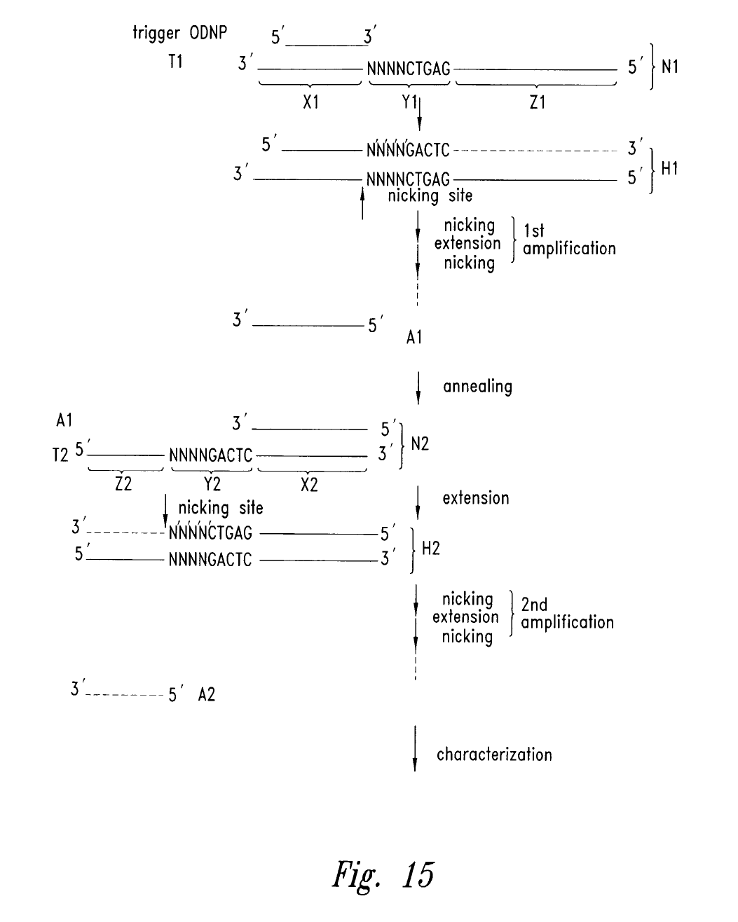

Another example of the embodiments where T2 comprises a sequence of an antisense strand of a nicking agent recognition sequence is illustrated in FIG. 15. In this example, the recognition sequence of N.BstNB I is used as an exemplary nicking agent recognition sequence. The amplification of A1 in the first amplification reaction is the same as that in FIG. 3, where the first template T1 comprises a sequence of the sense strand of the recognition sequence of N.BstNB I. The amplification of A2 in the second amplification reaction is the same as that in FIG. 14.

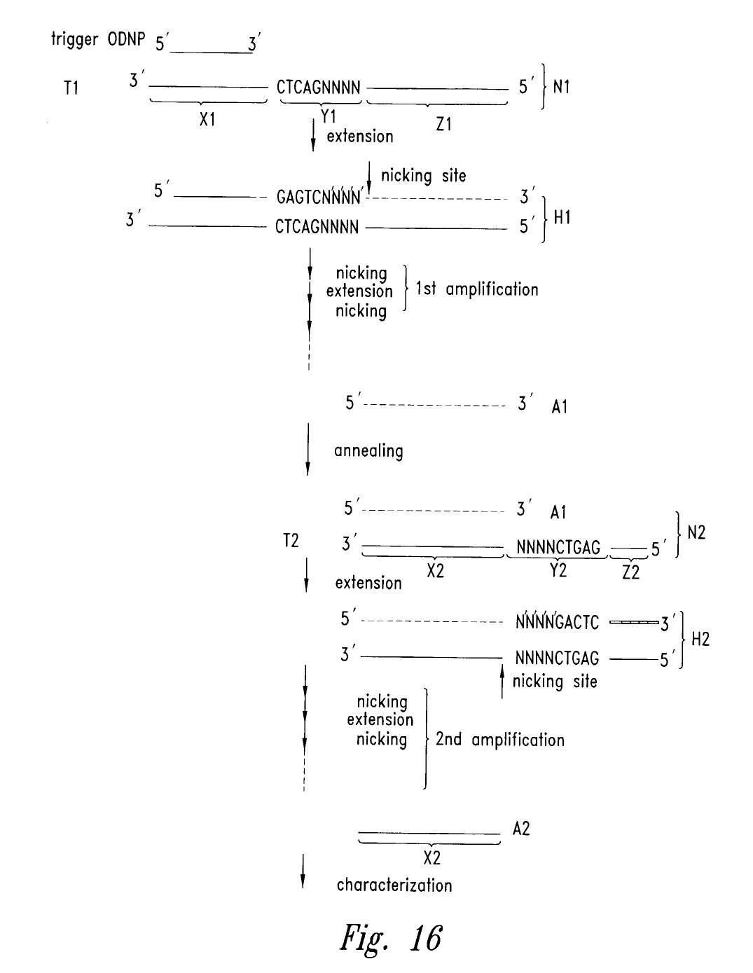

In certain other embodiments, a T2 molecule may comprise a sequence of the sense strand of a nicking agent recognition sequence. An example of such embodiments are shown in FIG. 16 using the recognition sequence of N.BstNB I as an exemplary nicking agent recognition sequence. In FIG. 16, the amplification of A1 is the same as that in FIG. 2, where T1 comprises a sequence of the antisense strand of a nicking agent recognition sequence. A1 is then used as an initial primer for the second amplification reaction. It is annealed to Region X2 of T2 , which also has two additional regions: Regions Y2 and Z2, to form an initial nucleic acid molecule N2 for the second amplification reaction. Region Y2 consists of a sequence of the sense strand of the recognition sequence of N.BstNB I and four nucleotides directly 3′ to the sequence (i.e., 3′-NNNNCTGAG-5′ where each of the Ns may be A, T, G, or C), whereas Regions X2 and Z2 refer to regions immediately next to the 3′ terminus and the 5′ terminus of Region Y2, respectively. The extension of A1 using T2 as a template provides an extension product (H2) that can be completely or partially double-stranded, depending on whether the 5′ terminal sequence of A1 anneals to the 3′ terminal sequence of Region X2. Because H2 comprises the double-stranded N.BstNB I recognition sequence, it can be nicked in the presence of N.BstNB I. The resulting 3′ terminus at the nicking site may be extended again by the DNA polymerase, which displaces Region X2. The nicking-extension cycle is repeated multiple times, resulting in the accumulation/amplification of a displaced strand A2 that contains the 5′ terminus at the nicking site. A2 is exactly identical to Region X2 if the 5′ terminal sequence of A1 anneals to the 3′ terminal sequence of Region X2. Otherwise, A2 and Region X2 is substantially complementary to each other as they have different lengths. The amplification of A2 is exponential because it is the final amplification product of two linked linear amplification reactions.

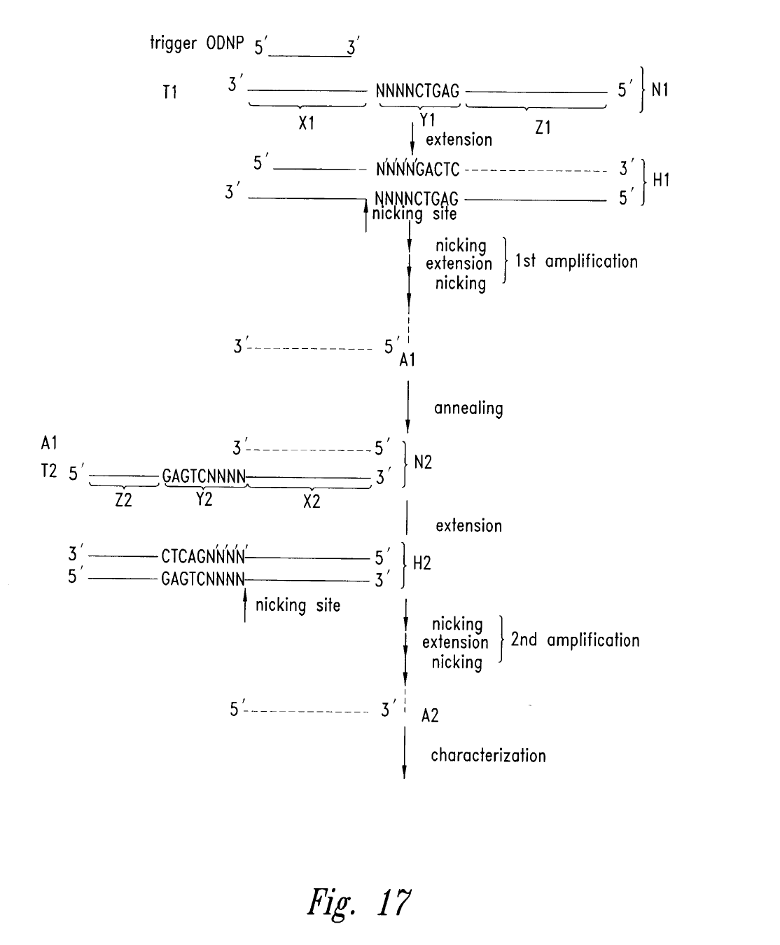

Another example of the embodiments where T2 comprises a sequence of a sense strand of a nicking agent recognition sequence is illustrated in FIG. 17. In this example, the recognition sequence of N.BstNB I is used as an exemplary nicking agent recognition sequence. The amplification of A1 in the first amplification reaction is the same as that in FIG. 3, where the first template T1 comprises a sequence of the sense strand of the recognition sequence of N.BstNB I. The amplification of A2 in the second amplification reaction is the same as that in FIG. 16.

In addition to the above exemplary embodiments, exponential nucleic acid amplification may be carried out by linking various linear amplification methods described in the sections related to gene expression analyses that perform linear amplification with a second linear amplification reaction. The single-stranded nucleic acid molecule amplified by the linear amplification reactions described in those sections may be annealed to a second template nucleic acid T2 that comprises the sequence of one strand of a nicking agent recognition sequence. The resulting initial nucleic acid N2 may be extended and used as a template for amplifying a second single-stranded nucleic acid molecule A2 .

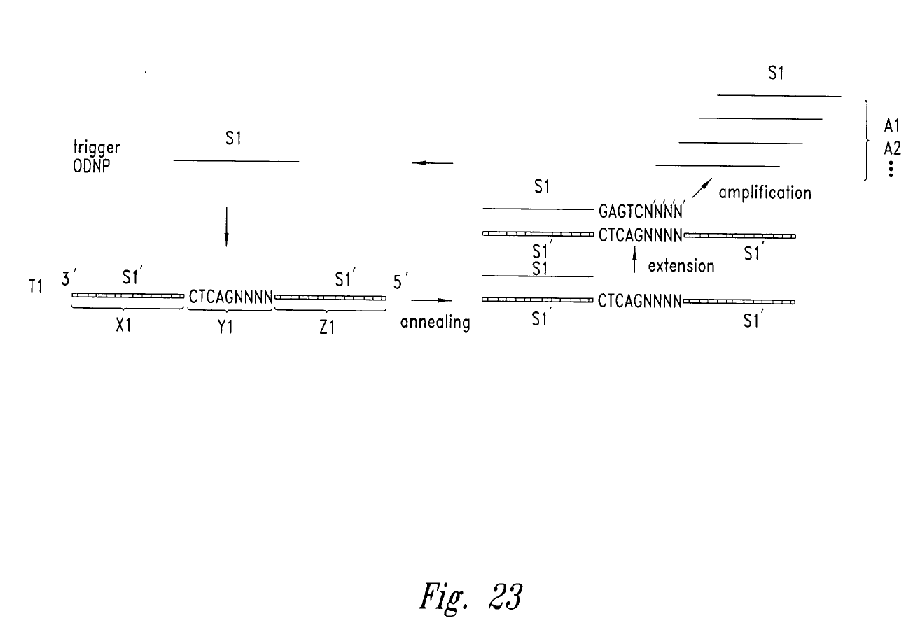

In some other embodiments, exponential nucleic acid amplification may be performed in the presence of only one template nucleic acid (i.e., a T1 molecule). For instance, in an embodiment using the recognition sequence of N.BstNB I as an exemplary recognition sequence shown in FIG. 23, Region X1 and Region Z1 of a T1 molecule may both comprise an identical sequence (referred to as “S1”) that is substantially or exactly complementary to the sequence of the trigger ODNP (referred to as “S1”). During the first amplification, because A1 is amplified using Region Z1 as a template, A1 has the same sequence as S1. A1 may then function as an oligonucleotide primer for a second amplification reaction using another molecule of T1 as a template. Because the oligonucleotide primer and the template for the first amplification reaction have sequences identical to those of the primer and the template for the second amplification reaction, respectively; the amplified nucleic acid fragment (A2) resulting from the second amplification reaction has the same sequence as that of the amplified nucleic acid fragment (A1) from the first amplification reaction. A2 may then function as an oligonucleotide primer for a third amplification reaction using another molecule of T1 as a template, amplifying a nucleic acid fragment (A3) that is identical to A2 . The above process may be repeated multiple times until all T1 molecules anneal to trigger ODNP molecules or amplified fragments (i.e., A1 , A2 , A3, etc.), or one of the other necessary components of the nucleic acid amplification reactions (e.g., deoxynucleoside triphosphates) is exhausted.

During the above-described nucleic acid amplification process, the presence of a trigger ODNP (derived from a target mRNA or cDNA) initiates multiple amplification reactions linked by an amplified nucleic acid fragment from a previous amplification reaction that functions as an amplification primer for a subsequent amplification reaction. Each reaction uses a T1 molecule as a template and amplifies a nucleic acid fragment with a sequence identical to the trigger ODNP. The end result is very rapid amplification of trigger ODNPs in the presence of template T1 molecules.

In some embodiments of one-template amplification of a trigger ODNP, Region X1 may contain an additional sequence other than a sequence (S1x′) that is at least substantially complementary to the sequence of a trigger ODNP (S1). The additional sequence may be between S1x′ and the sequence of the antisense strand of the NARS in T1 and contain no more than 5, 10, 15, 20, 25, 50, or 100 nucleotides. Likewise, Region Z1 may also contain an additional sequence other than a sequence (S1z′) that is at least substantially identical to S1x′. However, if such an additional sequence is present in Region Z1, S1z′ need be located at the 5′ terminus of T1 , unless it is complementary to Region Y1 or a 3′ portion thereof, so that no additional sequence is present at the 3′ terminus of A1 to prevent A1 from being extended using another T1 molecule as a template. In some embodiments, the additional sequence is present between the sequence of the antisense strand of the NARS in T1 and S1z′ and contain no more than 5, 10, 15, 20, 25, 50, or 100 nucleotides.

In certain embodiments of the above exponential amplification of a trigger ODNP, T1 may be at most 50, 75, 100, 150 or 200 nucleotides in length. In some embodiments, S1x′ and/or S1z′ are at least 6, 8, 10, 12, 14, 16, 18, or 20 nucleotides in length. In some preferred embodiments, S1x′ and/or S1z′ are 8 to 24, more preferably, 12 to 17 nucleotides in length.

As described above, the exponential nucleic acid method of the present invention links two or more nucleic acid amplification reactions together and each amplification reaction is performed in the presence of a nicking agent. The nicking agent for one amplification reaction may be different from that for another amplification reaction. Alternatively, the nicking agent for different amplification reactions may be identical to each other, so that only one nicking agent is required for exponential amplification of a nucleic acid molecule.

Likewise, the DNA polymerase of one amplification reaction may be different from that of another amplification reaction. Alternatively, the nicking agent for different amplification reactions may be identical to each other, so that only one DNA polymerase is required for exponential amplification of a nucleic acid molecule.

In certain embodiments, the second amplification reaction is performed under isothermal conditions. In some embodiments, both the first and second amplification reactions are performed under isothermal conditions.

In some embodiments, both the first and second amplification reactions are performed in a single vessel and thus performed under identical conditions. In such embodiments, the number of T2 molecules in an amplification reaction mixture is preferably, but is not required to be more than, that of T1 molecules. The preference for a greater number of T2 molecules than T1 molecules is due to the fact that T2 molecules are used as annealing partners for the single-stranded nucleic acid molecules A1 amplified using T1 molecules as templates. In other words, during the first amplification reaction, each T1 molecule is used as a template to produce multiple copies of A1 . Thus, for each of the T1 molecules, multiple T2 molecules are preferably present to provide annealing partners for the multiple A1 molecules amplified using a single T1 molecule as a template.

T2 molecules of the present invention may or may not be immobilized to a solid support. If immobilized, multiple T2 molecules on distinct areas of the solid support may form an array so that the second round of nucleic acid amplification is performed on the array. Such an array may be of a type similar to one of the arrays of the other nucleic acids of the present invention (e.g., a T1 array) described above.

In certain embodiments, the amplification product of the second amplification reaction may be relatively short and has at most 25, 20, 17, 15, 10, or 8 nucleotides. Such short length may be accomplished by appropriately designing T2 molecules. The short length of an A2 molecule may be advantageous because it increases amplification efficiencies and rates. In addition, it allows the use of a DNA polymerase that does not have a stand displacement activity. It also facilitates the detection of A2 molecules via certain technologies such as mass spectrometric analysis.

The present method of nucleic acid amplification is not limited to linking two nucleic acid amplification reactions together. In certain embodiments, a second amplification reaction may be further linked to a third amplification reaction. In other words, the nucleic acid molecule A2 amplified during the second amplification reaction may anneal to a portion of another nucleic acid molecule “T3” that comprises the sequence of one strand of a NARS to trigger the amplification of a nucleic acid molecule “A3” in a third amplification reaction. Additional amplification reactions may be added to the chain. For example, A3 may in turn anneal to a portion of another nucleic acid molecule “T4” also comprising one strand of a NARS and trigger the amplification of a nucleic acid molecule “A4” in a fourth amplification reaction. Because each subsequent amplification reaction results in a linear amplification of the amplified fragment from its previous amplification reaction, the greater number of the amplification reactions in an amplification system, the higher level of amplification, provided that the other components of the system (e.g., template nucleic acid molecules, NAs, and DNA polymerases) do not limit the amplification rate or level.

D. Compositions and Kits for Gene Expression Analyses

In an aspect, the present invention provides a nucleic acid molecule that comprises a sequence that is at least substantially identical to a portion of a naturally occurring genomic DNA or a cDNA of a naturally occurring mRNA having a sequence of the antisense strand of a double-stranded nicking agent recognition sequence. The nucleic acid is at most 200, 150, 120, 100, 75, 50, 40, 30, 25 or 20 nucleotides in length. It comprises from 3′ to 5′ three regions: Regions A, B and C. Region A is a nucleotide sequence that is at most 100, 75, 50, 40, 30, 25, 20, 15, 10, 8, 7, 6, 5, 4, or 3 nucleotides in length. Region B is the sequence of the antisense strand of the nicking agent recognition sequence present in the portion of the naturally occurring genomic DNA or the cDNA of the naturally occurring mRNA. Region C is a nucleotide sequence that is at most 100, 75, 50, 40, 30, 25, 20, 15, 10, 8, 7, 6, 5, 4, or 3 nucleotides in length. The nucleic acid may function as a template for detecting an mRNA or cDNA molecule that comprises a sequence of the sense strand of a double-stranded nicking agent recognition sequence as described above (e.g., FIG. 5).

In certain embodiments, the nucleic acid molecule of the present invention comprises a sequence that is exactly identical to a portion of a naturally occurring genomic DNA or a cDNA of a naturally occurring mRNA having a sequence of the antisense strand of a nicking agent recognition sequence. In other embodiments, the nucleic acid molecule comprises a sequence that is substantially identical to a portion of a naturally occurring genomic DNA or a cDNA of a naturally occurring mRNA having a sequence of the antisense strand of a nicking agent recognition sequence. The sequence of the nucleic acid molecule that is substantially identical to a portion of a naturally occurring genomic DNA or a cDNA of a naturally occurring mRNA may be at least 90%, 91%, 92%, 93%, 94%, 95%, 96%, 97%, 98%, or 99% identical to the portion of the naturally occurring genomic DNA or the cDNA of the naturally occurring mRNA. In this context, percent sequence identity of two nucleic acids is determined using BLAST programs of Altschul et al. (J. Mol. Biol. 215: 403-10, 1990) with their default parameters. These programs implement the algorithm of Karlin and Altschul (Proc. Natl. Acad. Sci. USA 87:2264-8, 1990) modified as in Karlin and Altschul (Proc. Natl. Acad. Sci. USA 90:5873-7, 1993). BLAST programs are available, for example, at the web site http://www.ncbi.nim.nih.ov.

The present invention also provides a single-stranded nucleic acid molecule that may function as a template in amplifying a single-stranded nucleic acid fragment in the presence of a target cDNA or a target mRNA and a nicking agent. The single-stranded nucleic acid molecule is at most 200, 150, 120, 100, 75, 50, 40, 30, 25 or 20 nucleotides in length, comprises a sequence of the antisense strand of a double-stranded nicking agent recognition sequence that recognizable by the nicking agent, and is substantially complementary to the target cDNA molecule or the target mRNA molecule.

In a related aspect, the present invention further provides a single-stranded nucleic acid molecule that when annealing to a target cDNA or a target mRNA, allows for the amplification of a portion of the target cDNA or the target mRNA in the presence of a nicking agent. The single-stranded nucleic acid molecule is at most 200, 150, 120, 100, 75, 50, 40, 30, 25 or 20 nucleotides in length, comprises a sequence of the sense strand of a double-stranded nicking agent recognition sequence that recognizable by the nicking agent, is substantially complementary to the target cDNA molecule or the target mRNA molecule.

The present invention also provides kits for gene expression analyses. Such kits may comprise one, two, several or all of the following components: (1) a template T1 molecule that comprises one strand of a double-stranded nicking agent recognition sequence; (2) a nicking agent (e.g., a NE or a RE); (3) a suitable buffer for the nicking agent (2); (4) a DNA polymerase; (5) a suitable buffer for the DNA polymerase (5); (6) dNTPs; (7) a modified dNTP; (8) a control template and/or control oligonucleotide primers for amplifying a template nucleic acid; (9) a chromatography column; (10) a buffer for performing chromatographic characterization or separation of nucleic acids; (11) a strand displacement facilitator (e.g., 1 M trehalose); (12) microtiter plates or microwell plates; (13) oligonucleotide standards (e.g., 6 mer, 7 mer, 8 mer, 12 mer and 16 mer) for liquid chromatography and/or mass spectrometry; and (14) an instruction booklet for using the kit. Detailed descriptions of many of the above components have been provided above.

In certain embodiments, the composition of the present invention does not contain a buffer specific to a NA or a buffer specific to a DNA polymerase. Instead, it contains a buffer suitable for both the nicking agent and the DNA polymerase. For instance, if N.BstNB I is the nicking agent and exo⁻Vent is the DNA polymerase, the nicking-extension buffer can be 0.5×N.BstNB I buffer and 1×exo⁻Vent Buffer.

For gene expression analyses that perform exponential nucleic acid amplification, the kit may further comprises one or more additional components that are used in a second amplification reaction. These components include: (1) a second nicking agent; (2) a second DNA polymerase; and (3) a second template nucleic acid molecule T2.

In a related aspect, the present invention provides compositions for gene expression analyses that perform exponential nucleic acid amplification. Such compositions generally comprise a combination of a first at least partially double-stranded nucleic acid molecule (N1 or H1 ) and a second at least partially double-stranded nucleic acid molecule (N2 or H2 ) designed to function, respectively, in the first and the second nucleic acid amplification reactions as described above (FIGS. 14-17).

The compositions of the present invention may be made by simply mixing their components or by performing reactions that result in the formation of the compositions. The kits of the present invention may be prepared by mixing some of their components or keep each of their components in an individual container.

E. Applications of the Present Invention

As discussed in detail herein above, the present invention provides methods and compositions for gene expression analyses using nicking agents. The present invention will find utility in a wide variety of applications wherein it is necessary to determine where a gene of interest is expressed in a biological sample and wherein it is desirable to compare two nucleic acid populations. Such applications include, but are not limited to, the identification and/or characterization of infectious organisms that cause infectious diseases in plants or animals, or are related to food safety, and the identification and/or characterization of genes associated with diseases in plants, animals or humans, or with desirable traits in plants or animals such as high crop yields, increased disease resistance, and high nutrition values.

For instance, the present invention is useful for detecting a pathogen in a biological sample of interest by detecting a pathogen-specific gene expression. Alternatively, it may be used to detect the expression of a gene known to be associated with a particular trait (e.g., disease resistance or susceptibility) and thus is useful for predicting the likelihood for a particular subject from which the sample was obtained to have the particular trait.

In addition, the present invention also provides methods for profiling cDNA populations. Comparison between the profiles of two cDNA populations may identify the cDNA molecules common to both cDNA populations and those present in one population but not the other. Such an identification helps the identification and/or characterization of nucleic acid molecules associated with a trait that is possessed by only one organism from which one cDNA population is isolated, but not the other organism from which the other cDNA population is prepared.

The following examples are provided by way of illustration and not limitation.

### EXAMPLES

### Example 1

### Exponential Amplification OF A Nucleic Acid Sequence

This example describes the exponential amplification of a specific nucleic acid sequence using a nicking restriction endonuclease and DNA polymerase.

The oligonucleotides used in this example were obtained from MWG Biotech (North Carolina) and their sequences are listed below with the sequence of the sense or the antisense strand of the N.BstNB I recognition sequence underlined:

Template No. 1 (T1):
3′-acaaggtcagcatccactcagacaaggtcagcatcca-5′
Template No. 2 (T2):
3′-acaaggtcagcatccactcagctacaaggtcagcatcca-5′
Trigger ODNP:
5′-tgttccagtcgtaggtgagtctgtt-3′

The following reaction mixture was assembled at room temperature:

75 ul water

10 ul 10×Thermopol buffer (from NEB (Beverly, Mass.

5 ul 10×N.BstNBI (from NEB)

5 ul T1 at 0.2 nanomoles/ul

5 ul TOP1 at 0.2 nanomoles/ul

The mixture was heated to 95° C. and then cooled to 50° C. and held at 50° C. for 10 minutes. After the incubation at 50° C., the following duplex (N1 ) was formed:

5′-tgttccagtcgtaggtgagtctgtt-3′
3′-acaaggtcagcatccactcagacaaggtcagcatcca-5′

The above mixture was diluted into a reaction mixture containing the following:

25 ul 10×Thermopol buffer (from NEB)

12.5 ul 10×N.BstNBI (from NEB)

0.5 ul of the duplex mixture described above

10 ul 25 mM dNTPs (from NEB)

100 ul 1 M trehalose (from Sigma (St. Louis, Mo.

25 units N.BstNBI nicking enzyme (from NEB)

5 units exo⁻Vent DNA polymerase (from NEB)

5 ul T2

102 ul water

The reaction was incubated at 60° C. for 15 minutes. After 15 minutes, 10 ul of the reaction was sampled and subjected to mass spectrometry.

During the incubation at 60°, the following duplex (H1) was filled in by the action of the DNA polymerase with “▾” indicating the nicking site of N.BstNB I:

▾
5′-tgttccagtcgtaggtgagtctgttccagtcgtaggt-3′
3′-acaaggtcagcatccactcagacaaggtcagcatcca-5′

The nicking enzyme cuts the upper strand of H1 and releases the fragment having the sequence 5′-ccagtcgtaggt-3′ (referred to as “A1 ”). As this fragment (i.e., A1) is made, the following duplex (N2) is formed in the 60° C. reaction mixture.

5′-ccagtcgtaggt-3′

3′-acaaggtcaccatccactcagctacaaggtcagcatcca-5′

The polymerase fills in the duplex to form the following fragment (H2 ):

```
                                                        ▾
          5′-ccagtcgtaggtgagtcgatgttccagtcgtaggt-3′
3′-acaaggtcaccatccactcagctacaaggtcagcatcca-5′
```

The N.BstNB I nicks the duplex and generate the fragment have the sequence 5′-ttccagtcgtaggt-3′ (referred to as “A2 ”), which can prime T2 to form the following partial double-stranded fragment:

5′-ttccagtcgtaggt-3′
3′-acaaggtcaccatccactcagctacaaggtcagcatcca-5′

The above partial double-stranded fragment is filled in by the DNA polymerase to form the following duplex:

5′-ttccagtcgtaggtgagtcgatgftccagtcgtaggt-3′

3′-acaaggtcaccatccactcagctacaaggtcagcatcca-5′

This duplex is then nicked by the N.BstNB I, generating the fragment 5′-ttccagtcgtaggt-3′ (i.e., A2). The nicking and extension process is repeated multiple times, resulting in amplification of A2 molecules.

The amplified fragment A2 has a predicted mass/charge profile as follows:

| Mass/charge value | Mass/charge |
|---|---|
| 4348.8 − 1 = 4347.8 | 1 |
| 2174.9 − 1 = 2173.9 | 2 |
| 1449.9 − 1 = 1448.9 | 3 |
| 1087.5 − 1 = 1086.5 | 4 |

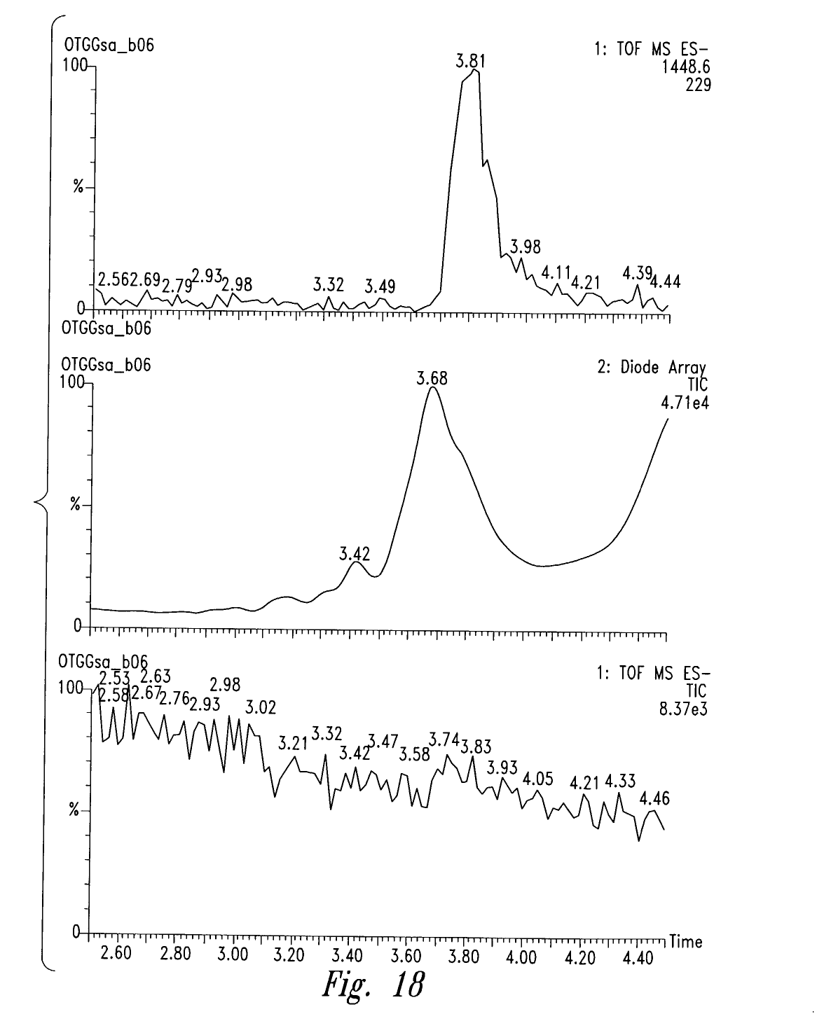

Mass spectrometry analyses of the amplified fragment A2 are shown in FIG. 18. The top panel shows the ion current for a fragment with a mass/charge ratio of 1448.6. The total ion current is 229 units. The middle panel shows the trace from the diode array. The bottom panel shows the total ion current from the mass spectrometer.

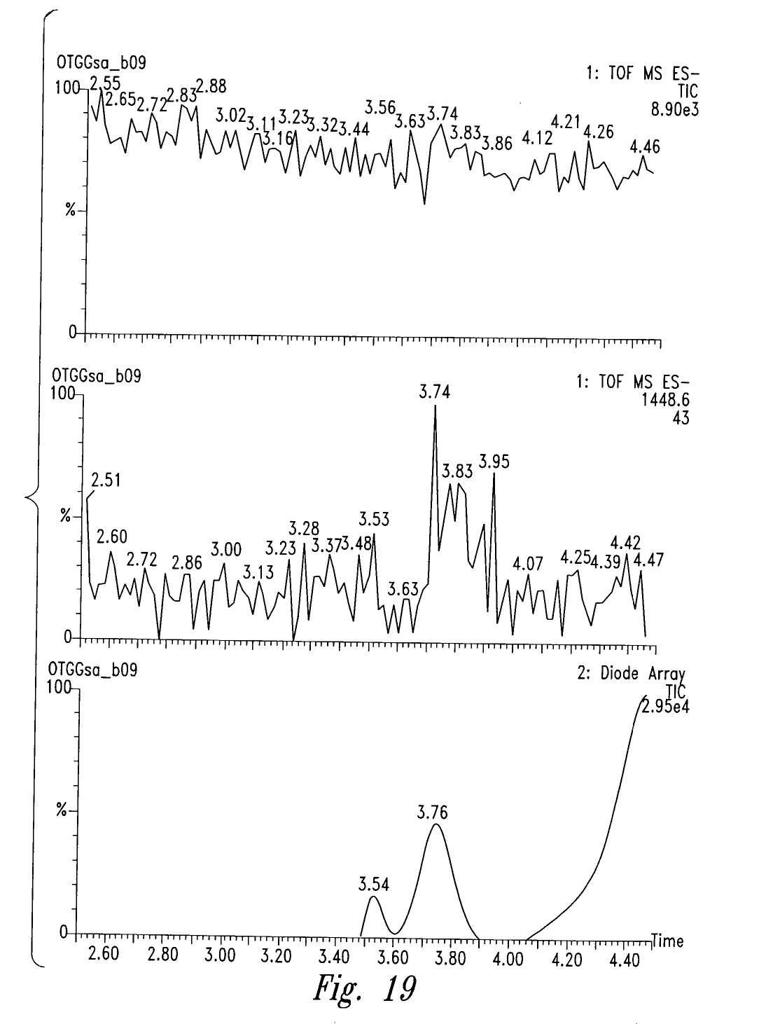

Mass spectrometry analyses in a control experiment are shown in FIG. 19. The top panel shows the total ion current from the mass spectrometer. The middle panel shows the ion current for a fragment with a mass/charge ratio of 1448.6. The total ion current is 43 units, which represents only background. The bottom panel shows the trace of diode array.

The above results indicate that there was exponential amplification of fragment A2 (10⁹ fold amplification was observed) and that no product was made in the control experiment in which TOP1 was omitted.

### Example 2

### Exponential Amplification Of An Oligonucleotide Using One Template

This example describes exponential amplification of an oligonucleotide using only one template nucleic acid.

The oligonucleotide sequences used in this example are as follows with the sequence of the antisense strand of the recognition sequence of N.BstNB I underlined:

Template (T1):
5′-cctacgactggaacagactcacctacgactgg a-3′
Trigger:
5′-ccagtcgtagg-3′

The above template and trigger form the following duplex when they anneal to each other

| | |
|---|---|
| Trigger: | 5′-ccagtcgtagg-3′ |
| Template: | 3′-aggtcagcatccactcagacaaggtcagcatcc-5′ |

In the presence of a DNA polymerase (e.g., exo⁻Vent or 9°Nm™), the above duplex is extended from the 3′ end of the trigger oligonucleotide to form the following extension product with the sequences of both strands of the recognition sequence of N.BstNB I underlined:

5′-ccagtcgtaggtgagtctgttccagtcgtagg-3′
3′-aggtcagcatccactcagacaaggtcagcatcc-5′

In the presence of N.BstNB I, the above extension product is nicked and produces a partially double-stranded nucleic acid and a single-stranded nucleic acid fragment (A1) having a sequence identical to that of the trigger oligonucleotide:

5′-ccagtcgtaggtgagtctgtt-3′ + 5′-ccagtcgtagg-3′
3′-aggtcagcatccactcagacaaggtcagcatcc-5′

The above extension and nicking may be repeated multiple times, resulting amplification of A1 molecules. In addition, A1 molecules may anneal to single-stranded T1 molecules, resulting additional amplification of A1 molecules.

The following reaction mixture was assembled at 4° C.

100 ul 10×Thermopol buffer

50 ul 10×N.BstNBI buffer

16 ul25 mM dNTPs

0.5 ul T1 at 100 pmol/ul

80 ul 2000 units/ml N.BstNBI (NEB)

24 ul 9°Nm™ DNA polymerase (NEB)

10 ul 400×SYBR (Molecular Probes, Eugene Wash.

740 ul water

The reaction mixture was thoroughly mixed at 4° C. 150 ul of the reaction mixture placed in a first tube, and 100 ul placed in 9 additional tubes. The trigger was diluted 100 times in water and then 1 ul placed in the first tube. Nine three-fold dilutions were then made.

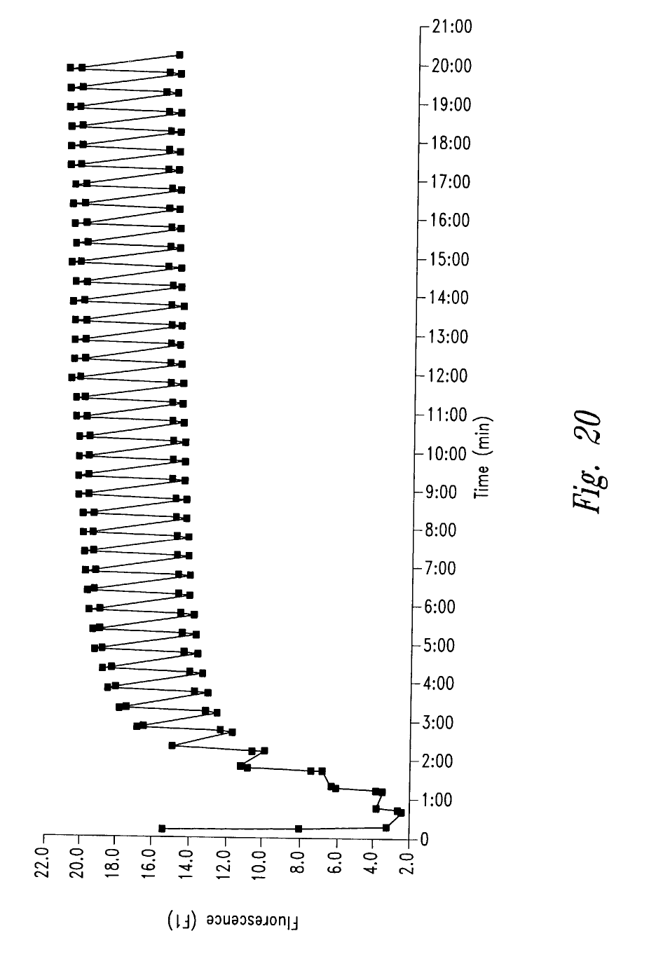

30 ul of each reaction was added to the light cycler capillaries. The capillaries were incubated at 60° C. for the indicated times. A representative result is shown in FIG. 20. This figure shows the accumulation of fluorescence in one of the light cycler capillaries as a function of time. The data are summarized in the following table:

| | Time to Maximum |
|---|---|
| Concentration of Trigger | Fluorescence |
| 3.3 × 10⁻³ picomoles/ul | 5 minutes |
| 1.1 × 10⁻³ picomoles/ul | 7 minutes |
| 3.7 × 10⁻⁴ picomoles/ul | 9 minutes |
| 1.2 × 10⁻⁴ picomoles/ul | 11 minutes |
| 4.1 × 10⁻⁵ picomoles/ul | 17 minutes |
| 1.4 × 10⁻⁵ picomoles/ul | 20 minutes |
| 4.5 × 10⁻⁶ picomoles/ul | 20 minutes |
| 1.5 × 10⁻⁶ picomoles/ul | 20 minutes |
| 5.0 × 10⁻⁷ picomoles/ul | 20 minutes |

The above result shows that there exists an approximate 20,000-fold range over which differences in starting concentrations of a trigger oligonucleotide can be measured and compared.

### Example 3

### Linear Amplification Of An Oligonucleotide

This example illustrates linear amplification of an oligonucleotide from a template duplex. The template duplex is formed by annealing two oligonucelotides to each other as shown below. The recognition sequence of N.BstNB I is shown below:

| | |
|---|---|
| ITATOP: | 5′-ccgatctagtgagtcgctc-3′ |
| NbBT16: | 3′-ggctagatcactcagcgagtcaaggtcagcatacc-5′ |

In the presence of a DNA polymerase, the recess of the above duplex is filled in to provide the following extension product:

5′-ccgatctagtgagtcgctcagttccagtcgtatgg-3′
3′-ggctagatcactcagcgagtcaaggtcagcatacc-5′

In the presence of N.BstNB I, the above extension product is nicked to produce the following nicked products:

5′-ccgatctagtgagtcgctc-3′ + 5′-agttccagtcgtatgg-3′
3′-ggctagatcactcagcgagtcaaggtcagcatacc-5′

The above extension and nicking cycle may be repeated multiple times, resulting in amplification of the fragment: 5′-agttccagtcgtatgg-3′. This fragment may be detected and characterized by liquid chromatography and mass spectrometry. It has a mass to charge ratio of 3 at 1663.1, a mass to charge ratio of 4 at 1247.1, and a mass to charge ratio of 5 at 997.1 daltons.

The following reaction was assembled at 4° C.:

740 ul deionized nuclease free water

110 ul 10×N.BstNB I buffer (NEB)

55 ul 10×N.BstNB I buffer (NEB)

1 ul of 1 picomole/ul of NBbt16 oligonucleotide

1 ul of 1 picomole/ul of ITATOP oligonucleotide

80 ul of 2000 units /ml of N.BstNB I (NEB)

24 ul of 5000 units/ml 9°Nm™ DNA polymease (NEB)

16 ul 25 mM dNTPs (NEB)

The reaction mixture was divided into 20 50 ul aliquots in PCR tubes. The tubes were placed at 60° C. on an MJ thermocycler and incubated for the indicated times. The samples were then subjected to the following liquid chromatography mass spectrometry analysis.

The column buffers are as follows: Buffer A contains 0.05 M dimethylbutylamine acetate, pH 7.6, while Buffer B contains 0.05 M dimethylbutylamine acetate, pH 7.6, 50% acetonitrile.

A shallow gradient of acetonitrile is used to elute the oligonucleotides and clean up the sample. The analysis portion of the gradient starts at 5% acetonitrile and increases to 15% over about 90 seconds, followed by a wash that quickly pushes a “plug” of 45% acetonitrile onto the column for just a few seconds followed by a return to starting conditions of 5% acetonitrile.

The column used is Guard column Xterra 2.×20 mm, 3.5 micron. MSC18. In front of the column is a frit in a frit holder (Upchurch A356 frit holder with Upchurch A701 Peek Prefilter Frit 0.5 micron).

The fractions from liquid chromatography were injected into mass spectrometer (Micromass LCT Time-of-Flight with an electrospray inlet, Micromass Inc., Manchester UK). The injection volume was 10 microliters. The samples were run electrospray negative mode with a scan range from 800 to 2000 amu. The time course results of the relative mass units at 1247.1 daltons are shown in the following table:

| Time | Relative Mass Units |
|---|---|
| 1 | 16 |
| 2 | 33 |
| 3 | 49 |
| 4 | 63 |
| 5 | 82 |
| 6 | 98 |
| 7 | 116 |
| 8 | 123 |
| 9 | 156 |
| 10 | 177 |
| 12.5 | 208 |
| 15 | 255 |
| 20 | 310 |
| 30 | 512 |
| 45 | 730 |
| 60 | 955 |
| 75 | 1233 |
| 90 | 1553 |

### Example 4

### Gene Expression Analysis Using Fluorescent Intercalating Agent For Detection

The following system permits the measurement of IL-1 mRNA or cDNA in any type of biological sample. A target cDNA is first generated from a biological sample, and subsequently triggers exponential amplification of a single-stranded oligonucleotide.

### IL-1 771 System

A cDNA fragment that contains a sequence of the sense strand of the recognition sequence N.BstNB I and a first template that is substantially complementary to the cDNA fragment are shown below. The sequences of the sense and antisense strands of the recognition sequence of N.BstNB I are underlined. 751 is the number of the first shown nucleotide of the IL-1 cDNA.

The cDNA fragment (only partial sequence shown):

751 5′- . . . tcaataacaagctggaatttgagtctgcccagftccccaac . . . -3′

The first template T1 :

771P 5′-ttggggaact gggcagactcaaattccagcttg-3′

The above cDNA fragment and the first template form the following duplex when they anneal to each other:

| | |
|---|---|
| 751 | 5′- . . . tcaataacaagctggaatttgagtctgcccagttccccaac . . . -3′ |
| 771P | 3′-gttcgaccttaaactcagacgggtcaaggggtt-5′ |

In the presence of N.BstNB I, the above duplex is nicked to produce the following nicked products:

| | |
|---|---|
| 751 | 5′- . . . tcaataacaagctggaatttgagtctgcc-3′ + 5′-cagttccccaac . . . -3′ |
| 771P | 3′-gttcgaccttaaactcagacgggtcaaggggtt-5′ |

In the presence of a DNA polymerase, the above partially double-stranded nicked product is extended to form the following extension product:

| | |
|---|---|
| 751 | 5′- . . . tcaataacaagctggaatttgagtctgcccagttccccaa-3′ |
| 771P | 3′-gttcgaccttaaactcagacgggtcaaggggtt-5′ |

The extension product may be re-nicked by N.BstNB I and produced the following nicked products:

| | |
|---|---|
| 751 | 5′- . . . tcaataacaagctggaatttgagtctgcc-3′ + 5′-cagttccccaa-3′ |
| 771P | 3′-gttcgaccttaaactcagacgggtcaaggggtt-5′ |

The partially double-stranded nicked product may be re-extended, and the extension product may be re-nicked. Such a nicking-extension cycle may be repeated multiple times, resulting in the amplification of the following oligonucleotide:

A1 5′-cagttccccaa-3′

The amplified oligonucleotide A1 may anneal to a second template T2 to form the following duplex:

| | |
|---|---|
| A1 | 5′-cagttccccaa-3′ |
| T2 | 3′-gtcaaggggttctcagatgcgtcaaggggtt-5′ |

The above duplex may be extended in the presence of the DNA polymerase to form the following extension product:

5′-cagttccccaagagtctacgcagttccccaa-3′
3′-gtcaaggggttctcagatgcgtcaaggggtt-5′

The above extension product may be nicked in the presence of the nicking agent to provide the following nicked products:

5′-cagttccccaagagtctacg-3′ + 5′-cagttccccaa-3′
3′-gtcaaggggttctcagatgcgtcaaggggtt-5′

The single-stranded oligonucleotide produced by the above nicking reaction has a sequence identical to that of A1 , thus is able to anneal to another T2 molecule and amplify itself.

### IL-1L 914 System

A cDNA fragment that contains a sequence of the sense strand of the recognition sequence N.BstNB I and a first template that is substantially complementary to the cDNA fragment are shown below. The sequences of the sense and antisense strands of the recognition sequence of N.BstNB I are underlined. 901 is the number of the first shown nucleotide of the IL-1 cDNA.

The cDNA fragment (only partial sequence shown):

901 5′- . . . agctgtacccagagagtcctgtgctgaatgtgg . . . -3′

The first template T1 :

914P 5′-ccacattcagcac aggactctct gggtacagct-3′

The above cDNA fragment and the first template form the following duplex when they anneal to each other:

| | |
|---|---|
| 901 | 5′-...agctgtacccagagagtcctgtgctgaatgtgg... |
| 914P | 3′-tcgacatgggtctctcaggacacgacttacacc-5′ |

In the presence of N.BstNB I, the above duplex is nicked to produce the following nicked products:

| | |
|---|---|
| 901 | 5′-...agctgtacccagagagtcctgt-3′ + 5′-gctgaatgtgg...-3′ |
| 914P | 3′-tcgacatgggtctctcaggacacgacttacacc-5′ |

In the presence of a DNA polymerase, the above partially double-stranded nicked product is extended to form the following extension product:

| | |
|---|---|
| 901 | 5′-...agctgtacccagagagtcctgtgctgaatgtgg-3′ |
| 914P | 3′-tcgacatgggtctctcaggacacgacttacacc-5′ |

The extension product may be re-nicked by N.BstNB I and produce the following nicked products:

| | |
|---|---|
| 901 | 5′-...agctgtacccagagagtcctgt-3′ + 5′-gctgaatgtgg-3′ |
| 914P | 3′-tcgacatgggtctctcaggacacgacttacacc-5′ |

The partially double-stranded nicked product may be re-extended, and the extension product may be re-nicked. Such a nicking-extension cycle may be repeated multiple times, resulting in the amplification of the following oligonucleotide:

A1 5′-gctgaatgtgg-3′

The amplified oligonucleotide A1 may anneal to a second template T2 to form the following duplex:

| | |
|---|---|
| A1 | 5′-gctgaatgtgg-3′ |
| T2 | 3′-cgacttacaccctcagatgccgacttacacc-5′ |

The above duplex may be extended in the presence of the DNA polymerase to form the following extension product:

5′-gctgaatgtgggagtctacggctgaatgtgg-3′
3′-cgacttacaccctcagatgccgacttacacc-5′

The above extension product may be nicked in the presence of N.BstNB I to provide the following nicked products:

5′-gctgaatgtgggagtctacg-3′ + 5′-gctgaatgtgg-3′
3′-cgacttacaccctcagatgccgacttacacc-5′

The single-stranded oligonucleotide produced by the above nicking reaction has a sequence identical to that of A1 , thus is able to anneal to another T2 molecule and amplify A1 itself.

### RNA Preparation and cDNA Synthesis

Total RNA was extracted by phenol-chloroform method and digested for 1 h at 37° C. with DNase I using the MessageClean kit (Gene Hunter, Nashville, Tenn. Poly(A)⁺ RNA was extracted by oligo(dT)-cellulose chromatography using the QIAGEN (Valencia, Calif. Oligotex mRNA Mini Kit. To synthesize cDNA from poly(A)⁺ RNA, 1 μg of poly(A)⁺ RNA was mixed with 1 μl of 10×CDS primer mix, incubated in a preheated thermal cycler at 70° C. for 2 min and at 50° C. for 2 min and then incubated at 50° C. for 25 min with a mixture of 5×reaction buffer, 10×dNTP, 0.5 μl of 100 mM dithiothreitol, and 50 units of Moloney murine leukemia virus reverse transcriptase (CLONTECH, Palo Alto, Calif. in a total volume of 10 μl. The reaction was stopped by adding 1 μl of 10×termination mix, and cDNA was purified on a Chroma Spin-200 column (CLONTECH).

The following reaction was assembled at 4° C.

100 ul 10×Thermopol buffer

50 ul 10×N.BstNBI buffer

16 ul25 mM dNTPs

0.5 ul T1 oligonucleotide at 100 pmol/ul

1.0 ul T2 oligonucleotide at 100 pmol/ul

80 ul 2000 units/ml N.BstNBI nicking enzyme (NEB)

24 ul 9°Nm™ DNA polymerase (NEB)

10 ul 400×SYBR (Molecular Probes, Eugene Wash.

740 ul water

The reaction was thoroughly mixed at 4° C. and then 150 ul placed in the first tube and 100 ul placed in the 9 additional tubes. The RNA was diluted 1 -100 times in 0.01 m Tris-HCl, 5 mM EDTA and then 1 ul placed in the first tube. Five 10-fold dilutions were then made.

25 ul of each reaction was added to the light cycler capillaries. The capillaries were incubated at 60° C. for 20 minutes. The data is summarized in the following tables:

| Concentration of | Fluorescence at 20 minutes |
|---|---|
| cDNA | for the IL-1 771 system |
| 4 × 10⁻⁴ ug/ul | 44.0 |
| 4 × 10⁻⁵ ug/ul | 22.4 |
| 4 × 10⁻⁶ ug/ul | 12.1 |
| 4 × 10⁻⁷ ug/ul | 6.3 |
| 4 × 10⁻⁸ ug/ul | 3.6 |
| none | 0.4 |

| | Fluorescence at 20 |
|---|---|
| Concentration of | minutes for the IL-1 914 |
| cDNA | system |
| 4 × 10⁻⁴ ug/ul | 36.9 |
| 4 × 10⁻⁵ ug/ul | 18.6 |
| 4 × 10⁻⁶ ug/ul | 8.9 |
| 4 × 10⁻⁷ ug/ul | 4.1 |
| 4 × 10⁻⁸ ug/ul | 2.4 |
| none | 0.4 |

### Example 5

### Gene Expression Analysis Using Liquid Chromatography And Mass Spectrometry For Detection

The templates for IL-1 771 and IL-1 914 systems are the same as those in Example 4. The following reaction was assembled on ice and placed on a preheated thermocycler at 60° C. for 10 minutes:

1×Thermopol buffer

0.5×N.BstNB I buffer

400 micromolar dNTPs

0.1 micromolar T2 oligonucleotide

0.2 micromolar T1 oligonucleotide

150 units/ml N.BstNB I

50 units 9°Nm™polymerase (NEB)

1×10⁻⁶ to 1×10⁻¹ pocomoles of cDNA converted from IL-1 mRNA

The sample was then subjected to liquid chromatography/mass spectrometry analysis as described in Example 3. The expected masses of the amplified fragments in the IL-1 771 and 914 systems are shown in the following table:

| IL-1 system | Mass/Charge 2 | Mass/Charge 3 | Mass/Charge 4 |
|---|---|---|---|
| 771 | 1669.2 | 1112.1 | 834.3 |
| 914 | 1745.1 | 1163.1 | 872.1 |

The results of exponential amplification of the IL-1 target are shown in the following table:

| Amount of starting material | 771 amplification | | 914 amplification | |
|---|---|---|---|---|
| 1 × 10⁻⁶ picomoles | 75 | RMU | 36 | RMU |
| 1 × 10⁻⁵ picomoles | 209 | RMU | 75 | RMU |
| 1 × 10⁻⁴ picomoles | 401 | RMU | 150 | RMU |
| 1 × 10⁻³ picomoles | 876 | RMU | 314 | RMU |
| 1 × 10⁻² picomoles | 1534 | RMU | 618 | RMU |
| 1 × 10⁻¹ picomoles | 3124 | RMU | 1334 | RMU |
| none | 0 | | 0 | |

### Example 6

### Nucleic Acid Amplification Using Template Nucleic Acid Comprising Mismatches In Nicking Agent Recognization Sequence

The following oligonucleotides were synthesized and obtained from MWG (MWG Biotech Inc., High Point, N.C. The oligonucleotides were placed in 0.01 M Tris-HCl and 0.001 M EDTA at 100 pmoles per microliter. The sequence of the sense strand of the double-stranded recognition sequence of N.BstNB I is underlined whereas the nucleotide(s) that is different from the nucleotide at the corresponding position(s) of the antisense strand of the double-stranded recognition sequence of N.BstNB I is italicized

| | |
|---|---|
| B-1: | 5′ CC TAC GAC TGG AAC AGA CTG ACC TAC GAC TGG A- 3′ |
| B-2: | 5′ CC TAC GAC TGG AAC AAT AAA ACC TAC GAC TGG A- 3′ |
| B-3: | 5′ CC TAC GAC TGG AAC AGA TTC ACC TAC GAC TGG A- 3′ |
| B-4: | 5′ CC TAC GAC TGG AAC AGA CAC ACC TAC GAC TGG A- 3′ |
| B-5: | 5′ CC TAC GAC TGG AAC AGT CTC ACC TAC GAC TGG A- 3′ |
| B-6: | 5′ CC TAC GAC TGG AAC AGA AAC ACC TAC GAC TGG A- 3′ |
| B-7: | 5′ CC TAC GAC TGG AAC AGT AAC ACC TAC GAC TGG A- 3′ |
| T-1: | 3′ GG ATG CTG ACC TTG TCT GAG TGG ATG CTG ACC T- 5′ |
| T-1a: | 3′ GG ATG CTG ACC TTG TCT GAG TGG ATG CTG ACC T- 5′ |
| T-1b: | 3′ GG ATG CTG ACC TTG TCT GAG TGG ATG CTG ACC - 5′ |
| T-1c: | 3′ GG ATG CTG ACC TTG TCT GAG TGG ATG CTG AC- 5′ |
| T-1d: | 3′ GG ATG CTG ACC TTG TCT GAG TGG ATG CTG A- 5′ |
| T-1e: | 3′ GG ATG CTG ACC TTG TCT GAG TGG ATG CTG - 5′ |
| T-1f: | 3′ GG ATG CTG ACC TTG TCT GAG TGG ATG CT- 5′ |
| T-1g: | 3′ GG ATG CTG ACC TTG TCT GAG TGG ATG C- 5′ |
| T-1h: | 3′ GG ATG CTG ACC TTG TCT GAG TGG ATG- 5′ |
| T-1i: | 3′ GG ATG CTG ACC TTG TCT GAG TGG AT- 5′ |
| T-1j: | 3′ GG ATG CTG ACC TTG TCT GAG TGG A- 5′ |
| T-1k: | 3′ GG ATG CTG ACC TTG TCT GAG TGG - 5′ |
| T-1l: | 3′ GG ATG CTG ACC TTG TCT GAG TG- 5′ |
| T-1m: | 3′ GG ATG CTG ACC TTG TCT GAG T- 5′ |
| T-1n: | 3′ GG ATG CTG ACC TTG TCT GAG - 5′ |

The following mixture was combined and then 25 microliters of the mixture was added to each well in the microtiter plate.

250 ul 10×Thermopol buffer (NEB Biolabs, Beverly, Mass.

125 ul 10×N.BstNBI (NEB Biolabs, Beverly, Mass.

100 ul 25 mM dNTPs (NEB Biolabs, Beverly, Mass.

1000 ul 1 M trehalose (Sigma, St. Louis, Mo.

250 units N.BstNBI nicking enzyme.(NEB Biolabs, Beverly, Mass.

50 units Vent exo- DNA polymerase (NEB Biolabs, Beverly, Mass.

1020 ul ultra pure water

25 microliters of each respective duplex was then added to the microtiter plate. The duplex was formed by first diluting two oligonucleotide primers and placing them in the following solution at a final concentration of 1 pmole per microliter: 1×Thermopol buffer (New England Biolabs, Beverly, Mass. and 0.5×N.BstNBI buffer. The 1×Thermopol buffer consists of 10 mM KCl, 10 mM (NH₄)₂SO₄, 20 mM Tris-HCl pH8.8, 0.1% Triton X-100, 2 mM MgSO₄, whereas the 1×N.BstNBI buffer consists of 150 mM KCl, 10 mMTris-HCl, 10 mM MgCl₂, 1 mM DTT. The mixture was then heated to 100° C. for 1 minute and then held at 50° C. for 10 minutes to allow the duplexes to form. The plate was resealed at 4° C., and then heated to 60° C. for 1 hour.

The following duplexes were tested:

| #1 (perfect base pairing) | |
|---|---|
| T-1: | 3′ GG ATG CTG ACC TTG TCT GAG TGG ATG CTG ACC T- 5′ |
| B-1: | 5′ CC TAC GAC TGG AAC AGA CTC ACC TAC GAC TGG A- 3′ |
| #2 (complete mismatching) | |
| T-1: | 3′ GG ATG CTG ACC TTG TCT GAG TGG ATG CTG ACC T- 5′ |
| B-2: | 5′ CC TAC GAC TGG AAC AAT AAA ACC TAC GAC TGG A- 3′ |
| #3 (single mismatch) | |
| T-1: | 3′ GG ATG CTG ACC TTG TCT GAG TGG ATG CTG ACC T- 5′ |
| B-3: | 5′ GG TAC GAC TGG AAC AGA TTC ACC TAC GAC TGG A- 3′ |
| #4 (single mismatch) | |
| T-1: | 3′ GG ATG CTG ACC TTG TCT GAG TGG ATG CTG ACC T- 5′ |
| B-4: | 5′ CC TAC GAC TGG AAC AGA CAC ACC TAC GAC TGG A- 3′ |
| #4 (single mismatch) | |
| T-1: | 3′ GG ATG CTG ACC TTG TCT GAG TGG ATG CTG ACC T- 5′ |
| B-5: | 5′ CC TAC GAC TGG AAC AGT CTC ACC TAC GAC TGG A- 3′ |
| #6 (2 mismatches) | |
| T-1: | 3′ GG ATG CTG ACC TTG TCT GAG TGG ATG CTG ACC T- 5′ |
| B-6: | 5′ CC TAC GAC TGG AAC AGA AAC ACC TAC GAC TGG A- 3′ |
| #7 (3 mismatches) | |
| T-1: | 3′ GG ATG CTG ACC TTG TCT GAG TGG ATG CTG ACC T- 5′ |
| B-7: | 5′ CC TAC GAC TGG AAC AGT AAC ACC TAC GAC TGG A- 3′ |
| #8a. | |
| T-1: | 3′ GG ATG CTG ACC TTG TCT GAG TGG ATG CTG ACC T- 5′ |
| B-7: 5′ CC TAC GAC TGG AAC AGT AAC ACC TAC GAC TGG A- 3′ | |
| #8b. | |
| T-1: | 3′ GG ATG CTG ACC TTG TCT GAG TGG ATG CTG ACC - 5′ |
| B-7: | 5′ CC TAC GAC TGG AAC AGT AAC ACC TAC GAC TGG A- 3′ |
| #8c. | |
| T-1: | 3′ GG ATG CTG ACC TTG TCT GAG TGG ATC CTG AC- 5′ |
| B-7: | 5′ CC TAC GAC TGG AAC AGT AAC ACC TAC GAC TGG A- 3′ |
| #8d. | |
| T-1: | 3′ GG ATG CTG ACC TTG TCT GAG TGG ATG CTG A- 5′ |
| B-7: | 5′ CC TAC GAC TGG AAC AGT AAC ACC TAC GAC TGG A- 3′ |
| #8e. | |
| T-1: | 3′ GG ATG CTG ACC TTG TCT GAG TGG ATG CTG - 5′ |
| B-7: | 5′ CC TAC GAC TGG AAC AGT AAC ACC TAC GAC TGG A- 3′ |
| #8f. | |
| T-1: | 3′ GG ATG CTG ACC TTG TCT GAG TGG ATG CT- 5′ |
| B-7: | 5′ CC TAC GAC TGG AAC AGT AAC ACC TAC GAC TGG A- 3′ |
| #8g. | |
| T-1: | 3′ GG ATG CTG ACC TTG TCT GAG TGG ATG C- 5′ |
| B-7: | 5′ CC TAC GAC TGG AAC AGT AAC ACC TAC GAC TGG A- 3′ |
| #8h. | |
| T-1: | 3′ GG ATG CTG ACC TTG TCT GAG TGG ATG - 5′ |
| B-7: | 5′ CC TAC GAC TGG AAC AGT AAC ACC TAC GAC TGG A- 3′ |
| #8i. | |
| T-1: | 3′ GG ATG CTG ACC TTG TCT GAG TGG AT- 5′ |
| B-7: | 5′ CC TAC GAC TGG AAC AGT AAC ACC TAC GAC TGG A- 3′ |
| #8j. | |
| T-1: | 3′ GG ATG CTG ACC TTG TCT GAG TGG A- 5′ |
| B-7: | 5′ CC TAC GAC TGG AAC AGT AAC ACC TAC GAC TGG A- 3′ |
| #8k. | |
| T-1: | 3′ GG ATG CTG ACC TTG TCT GAG TGG - 5′ |
| B-7: | 5′ CC TAC GAC TGG AAC AGT AAC ACC TAC GAC TGG A- 3′ |
| #8l. | |
| T-1: | 3′ GG ATG CTG ACC TTG TCT GAG TG- 5′ |
| B-7: | 5′ CC TAC GAC TGG AAC AGT AAC ACC TAC GAC TGG A- 3′ |
| #8m. | |
| T-1: | 3′ GG ATG CTG ACC TTG TCT GAG T- 5′ |
| B-7: | 5′ CC TAC GAC TGG AAC AGT AAC ACC TAC GAC TGG A- 3′ |
| #8n. | |
| T-1: | 3′ GG ATG CTG ACC TTG TCT GAG - 5′ |
| B-7: | 5′ CC TAC GAC TGG AAC AGT AAC ACC TAC GAC TGG A- 3′ |
| #9a. | |
| T-1: | 3′ GG ATG CTG ACC TTG TCT GAG TGG ATG CTG ACC T- 5′ |
| B-7: | 5′ CC TAC GAC TGG AAC AAT AAA ACC TAC GAC TGG A- 3′ |
| #9b. | |
| T-1: | 3′ GG ATG CTG ACC TTG TCT GAG TGG ATG CTG ACC - 5′ |
| B-7: | 5′ CC TAC GAC TGG AAC AAT AAA ACC TAC GAC TGG A- 3′ |
| #9c. | |
| T-1: | 3′ GG ATG CTG ACC TTG TCT GAG TGG ATG CTG AC- 5′ |
| B-7: | 5′ CC TAC GAC TGG AAC AAT AAA ACC TAC GAC TGG A- 3′ |
| #9d. | |
| T-1: | 3′ GG ATG CTG ACC TTG TCT GAG TGG ATG CTG A- 5′ |
| B-7: | 5′ CC TAC GAC TGG AAC AAT AAA ACC TAC GAC TGG A- 3′ |
| #9e. | |
| T-1: | 3′ GG ATG CTG ACC TTG TCT GAG TGG ATG CTG - 5′ |
| B-7: | 5′ CC TAC GAC TGG AAC AAT AAA ACC TAC GAC TGG A- 3′ |
| #9f. | |
| T-1: | 3′ GG ATG CTG ACC TTG TCT GAG TGG ATG CT- 5′ |
| B-7: | 5′ CC TAC GAC TGG AAC AAT AAA ACC TAC GAC TGG A- 3′ |
| #9g. | |
| T-1: | 3′ GG ATG CTG ACC TTG TCT GAG TGG ATG C- 5′ |
| B-2: | 5′ CC TAC GAC TGG AAC AAT AAA ACC TAC GAC TGG A- 3′ |
| #9h. | |
| T-1: | 3′ GG ATG CTG ACC TTG TCT GAG TGG ATG - 5′ |
| B-2: | 5′ CC TAC GAC TGG AAC AAT AAA ACC TAC GAC TGG A- 3′ |
| #9i. | |
| T-1: | 3′ GG ATG CTG ACC TTG TCT GAG TGG AT- 5′ |
| B-2: | 5′ CC TAC GAC TGG AAC AAT AAA ACC TAC GAC TGG A- 3′ |
| #9j. | |
| T-1: | 3′ GG ATG CTG ACC TTG TCT GAG TGG A- 5′ |
| B-2: | 5′ CC TAC GAC TGG AAC AAT AAA ACC TAC GAC TGG A- 3′ |
| #9k. | |
| T-1: | 3′ GG ATG CTG ACC TTG TCT GAG TGG - 5′ |
| B-2: | 5′ CC TAC GAC TGG AAC AAT AAA ACC TAC GAC TGG A- 3′ |
| #9l. | |
| T-1: | 3′ GG ATG CTG ACC TTG TCT GAG TG- 5′ |
| B-2: | 5′ CC TAC GAC TGG AAC AAT AAA ACC TAC GAC TGG A- 3′ |
| #9m. | |
| T-1: | 3′ GG ATG CTG ACC TTG TCT GAG T- 5′ |
| B-2: | 5′ CC TAC GAC TGG AAC AAT AAA ACC TAC GAC TGG A- 3′ |
| #9n. | |
| T-1: | 3′ GG ATG CTG ACC TTG TCT GAG - 5′ |
| B-2: | 5′ CC TAC GAC TGG AAC AAT AAA ACC TAC GAC TGG A- 3′ |

The plate was loaded onto the LC/MS (Micromass LTD, Manchester UK and Beverly, Mass. USA) that is a LCT time-of-flight using electrospray in the negative mode. The conditions were as follows:

The chromatography system was an Agilent HPLC-1100 composed of a binary pump, degasser, a column oven, a diode array detector, and thermostatted microwell plate autoinjector (Palo Alto, Calif. The column was a Waters Xterra, incorporating C18 packing with 3 uM particle size, with 300 Angstrom pore size, 2.1 mm×50 mm (Waters Inc. Milford, Mass. The column was run at 30C. with a gradient of acetonitrile in 5 mM Triethylamine acetate (TEAA). Buffer A was 5 mM TEAA, buffer B was 5 mM TEAA and 25% (VN) acetonitrile. The gradient began with a hold at 10% B for one minute then ramped to 50% B over 4 minutes followed by 30 seconds at 95% B and finally returned to 10% B for a total run time of six minutes. The column temperature was held constant at 30C. The flow rate was 0.416 ml per minute. The injection volume was 10 microliters. Flow into the mass spectrometer was 200ul/min, half the LC flow was diverted to waste using a tee. The mass spectrometer was a Micromass LCT Time-of-Flight with an electrospray inlet (Micromass Inc. Manchester UK). The samples were run in electrospary negative mode with a scan range from 700 to 2300 amu using a 1 second scan time. Instrument parameters were: TDC start voltage 700, TDC stop voltage 50, TDC threshold 0, TDC gain control 0, TDC edge control 0, Lteff 1117.5, Veff 4600. Source parameters: Desolvation gas 862 L/hr, Capillary 3000V, Sample cone 25V, RF lens 200V, extraction cone 2V, desolvation temperature 250C., Source temperature 150C, RF DC offset 14V, FR DC offset 21V, Aperture 6V, accelaration 200V, Focus, 10 V, Steering OV, MCP detector 2700V, Pusher cycle time (manual) 60, Ion energy 40V, Tube lens OV, Grid 274V, TOF flight tube 4620V, Reflectron 1790V.

The following extracted ion currents were monitored: 1144.7 daltons plus or minus 1 dalton around 1144.7 for the following fragment to be released:

3′ GG ATG CTG ACC-5′

from the following duplex, as well as the other duplexes listed above:

| | |
|---|---|
| T-1: | 3′ GG ATG CTG ACC TTG TCT GAG TGG ATG CTG ACC T- 5′ |
| B-1: | 5′ CC TAC GAC TGG AAC AGA CTC ACC TAC GAC TGG A- 3′ |

| | Number of Mismatches Within | |
|---|---|---|
| Duplex | Double-Stranded N.BstNB1 | Relative Mass |
| Names | Recognition Sequence | Units Observed |
| 1 | 0 | 121.0 |
| 2 | 5 | 18.5 |
| 3 | 1 | 66.7 |
| 4 | 1 | 61.5 |
| 5 | 1 | 63.0 |
| 6 | 2 | 45.0 |
| 7 | 3 | 21.2 |
| 8a | 3 | 23.4 |
| 8b | 3 | 28.3 |
| 8c | 3 | 11.5 |
| 8d | 3 | 29.2 |
| 8e | 3 | 14.6 |
| 8f | 3 | 17.8 |
| 8g | 3 | 20.8 |
| 8h | 3 | 12.3 |
| 8i | 3 | 14.9 |
| 8j | 3 | 18.3 |
| 8k | 3 | 19.3 |
| 8l | 3 | 15.6 |
| 8m | 3 | 18.3 |
| 8n | 3 | 12.5 |
| 9a | 5 | 21.3 |
| 9b | 5 | 17.8 |
| 9c | 5 | 19.2 |
| 9d | 5 | 15.3 |
| 9e | 5 | 14.0 |
| 9f | 5 | 15.9 |
| 9g | 5 | 28.3 |
| 9h | 5 | 22.7 |
| 9i | 5 | 23.9 |
| 9j | 5 | 21.4 |
| 9k | 5 | 22.6 |
| 9l | 5 | 22.5 |
| 9m | 5 | 13.5 |
| 9n | 5 | 14.3 |

The results are shown in the table below:

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is

1. A method for determining the presence or absence of a target cDNA molecule in a cDNA population or for determining the presence or absence of a target mRNA molecule in a biological sample, comprising:
(A) forming a mixture comprising:
(i) the cDNA molecules of the cDNA population or the RNA molecules of the biological sample,
(ii) a template nucleic acid molecule that
(a) comprises one strand of a nicking agent recognition sequence, and
(b) is at least substantially complementary to the target cDNA if the target cDNA is single-stranded,
is at least substantially complementary to one strand of the target cDNA if the target cDNA is double-stranded, or
is at least substantially complementary to the target mRNA,
(iii) a nicking agent that recognizes the recognition sequence,
(iv) a DNA polymerase, and
(v) one or more deoxynucleoside triphosphate(s);
(B) maintaining the mixture at conditions that amplify a single-stranded nucleic acid molecule using a portion of the target cDNA, a portion of the target mRNA, or a portion of the template nucleic acid molecule as a template, if the target cDNA is present in the cDNA population or if the target mRNA is present in the biological sample; and
(C) detecting the presence or absence of the single-stranded nucleic acid molecule to determine the presence or absence of the target cDNA molecule in the cDNA population, or to determine the presence or absence of the target mRNA in the biological sample.

2. The method of claim 1 wherein the template nucleic acid comprises a sequence, located 3′ to the sequence of the one strand of the nicking agent recognition sequence, that is at least substantially complementary to the 3′ portion of the target cDNA if the target cDNA is single-stranded, to the 3′ portion of one strand of the target cDNA if the target cDNA is double-stranded, or to the target mRNA.

3. The method of claim 1 wherein the target cDNA is double-stranded and comprises the nicking agent recognition sequence, and wherein the template nucleic acid comprises the portion of the target cDNA that contains the sequence of the antisense strand of the nicking agent recognition sequence.

4. The method of claim 1 wherein the target cDNA is single-stranded and comprises the sequence of the sense strand of the nicking agent recognition sequence, and wherein the template nucleic acid comprises the sequence of the antisense strand of the nicking agent recognition sequence.

5. The method of claim 1 wherein the target cDNA is double-stranded and comprises the nicking agent recognition sequence, and wherein the template nucleic acid comprises, from 3′ to 55′:
(i) a sequence that is at least substantially complementary to the strand of the target cDNA that comprises the sequence of the sense strand of the nicking agent recognition sequence,
(ii) the sequence of the antisense strand of the nicking agent recognition sequence, and
(iii) a sequence that is not substantially complementary to the strand of the target cDNA that comprises the sequence of the sense strand of the nicking agent recognition sequence.

6. The method of claim 1 wherein the target cDNA is single-stranded and comprises the sequence of the sense strand of the nicking agent recognition sequence, and wherein the template nucleic acid comprises, from 3′ to 55′:
(i) a sequence that is at least substantially complementary to the target cDNA,
(ii) the sequence of the antisense strand of the nicking agent recognition sequence, and
(iii) a sequence that is not substantially complementary to the target cDNA.

7. The method of claim 1 wherein the target cDNA is immobilized.

8. The method of claim 1 wherein the template nucleic acid molecule comprises the sequence of the sense strand of the nicking agent recognition sequence.

9. The method of claim 1 wherein the template nucleic acid molecule comprises the sequence of the antisense strand of the nicking agent recognition sequence.

10. The method of claim 8 wherein one or more nucleotides in the sequence of the sense strand of the nicking agent recognition sequence do not form a conventional base pair with nucleotides of the target cDNA or the target mRNA.

11. The method of claim 1 wherein the cDNA molecules or the cDNA population or the RNA molecule of the biological sample are immobilized to a solid support.

12. The method of claim 1 wherein the template nucleic acid molecule is immobilized to a solid support.

13. The method of claim 9 wherein a sequence located 5′ to the sequence of the antisense strand of the nicking agent recognition sequence is at least substantially identical to a sequence located 5′ to the sequence of the antisense strand of the nicking agent recognition sequence.

14. The method of claim 13 wherein the sequence located 5′ to the sequence of the antisense strand of the nicking agent recognition sequence is exactly identical to the sequence located 5′ to the sequence of the antisense strand of the nicking agent recognition sequence.

15. The method of claim 13 wherein the sequence located 5′ to the sequence of the antisense strand of the nicking agent recognition sequence is at least 10 nucleotides.

16. The method of claim 1 wherein the nicking agent is a nicking endonuclease.

17. The method of claim 16 wherein the nicking endonuclease is N.BstNB I.

18. The method of 1 wherein the nicking agent is a restriction endonuclease.

19. The method of 1 wherein step (B) is performed under isothermal conditions.

20. The method of claim 19 wherein step (B) is performed at 50° C.-70° C.

21. The method of claim 20 wherein step (B) is performed at 60° C.

22. The method of claim 1 wherein the DNA polymerase is 5′→3′ exonuclease deficient.

23. The method of claim 22 wherein the 5′→3′ exonuclease deficient DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9°Nm™ DNA polymerase and T4 DNA polymerase homoenzyme.

24. The method of claim 23 wherein the 5′→3′ exonuclease deficient DNA polymerase is exo−Bst polymerase, exo⁻ Bca polymerase, exo⁻ Vent polymerase, 9°Nm™ DNA polymerase or exo⁻ Deep Vent polymerase.

25. The method of claim 1 wherein the DNA polymerase has a strand displacement activity.

26. The method of claim 1 wherein the DNA polymerase does not have a strand displacement activity.

27. The method of claim 1 wherein step (B) is performed in the presence of a strand displacement facilitator.

28. The method of claim 27 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

29. The method of claim 28 wherein the strand displacement facilitator is trehalose.

30. The method of claim 1 wherein the single-stranded nucleic acid molecule of step (B) has at most 24 nucleotides.

31. The method of claim 1 wherein the single-stranded nucleic acid molecule of step (B) has at most 20 nucleotides.

32. The method of claim 1 wherein the single-stranded nucleic acid molecule of step (B) has at most 17 nucleotides.

33. The method of claim 1 wherein the single-stranded nucleic acid molecule of step (B) has at most 12 nucleotides.

34. The method of claim 1 wherein the single-stranded nucleic acid molecule of step (B) has at most 8 nucleotides.

35. The method of claim 1 wherein step (C) is performed at least partially by the use of a technology selected from the group consisting of mass spectrometry, liquid chromatography, fluorescence polarization, nucleic acid hybridization, and electrophoresis.

36. The method of claim 1 wherein step (C) is performed at least partially by the use of liquid chromatography.

37. The method of claim 1 wherein step (C) is performed at least partially by the use of mass spectrometry.

38. The method of claim 1 wherein step (C) is performed at least partially by the use of liquid chromatography and mass spectrometry.

39. A method for determining the presence or absence of an mRNA in a sample, comprising:
(a) synthesizing single-stranded cDNA molecules using the mRNA molecules in the sample as templates;
(b) forming a mixture comprising:
(i) the single-strand cDNA molecules from step (a),
(ii) a single-stranded nucleic acid probe that comprises, from 3′ to 5′, a sequence that is at least substantially complementary to the 3′ portion of the target nucleic acid, and a sequence of the antisense strand of a nicking agent recognition sequence;
(c) removing unhybridized probe from the mixture of step (b);
(d) performing an amplification reaction in the presence of a nicking agent that recognizes the nicking agent recognition sequence; and
(e) detecting and/or characterizing the presence or absence of the amplification product of step (d) to determine the presence or absence of the target nucleic acid in the sample.

40. The method of claim 39 wherein the 5′ termini of the single-stranded cDNA molecules are immobilized.

41. The method of claim 40 wherein step (a) is performed using an immobilized oligonucleotide primer.

42. A method for determining the presence or absence of a double-stranded target cDNA molecule that comprises a nicking agent recognition sequence in a cDNA population, comprising:
(A) forming a mixture comprising the cDNA population, a nicking agent that recognizes the nicking agent recognition sequence, a DNA polymerase, and one or more deoxynucleoside triphosphate(s);
(B) maintaining the mixture at conditions that amplify a single-stranded nucleic acid molecule using one strand of the target cDNA molecule as a template, if the target cDNA molecule is present in the cDNA population; and
(C) detecting the presence or absence of the single-stranded nucleic acid fragment amplified in step (B) to determine the presence or absence of the target cDNA.

43. The method of claim 42 wherein the cDNA population is digested with a restriction endonuclease before being mixed with the nicking agent, the DNA polymerase, and the one or more deoxynucleoside triphosphate(s) in step (A).

44. A method for profiling a cDNA population comprising:
(A) forming a mixture comprising the cDNA population, a nicking agent, a DNA polymerase, and one or more deoxynucleoside triphosphate(s);
(B) maintaining the mixture at conditions that amplify single-stranded nucleic acid molecules using the cDNA molecules that comprise a recognition sequence of the nicking agent as templates; and
(C) characterizing the single-stranded nucleic acid fragments to profile the cDNA population.

45. The method of claim 44 wherein the cDNA population is digested with a restriction endonuclease before being mixed with the nicking agent, the DNA polymerase, and the one or more deoxynucleoside triphosphate(s) in step (A).

46. The method of claim 44 wherein the nicking agent is a nicking endonuclease.

47. The method of claim 46 wherein the nicking agent is N.BstNB I.

48. The method of claim 44 wherein the DNA polymerase is selected from exo⁻ Bst polymerase, exo⁻ Bca polymerase, exo⁻ Vent polymerase, 9°Nm™DNA polymerase, and exo⁻ Deep Vent polymerase.

49. The method of claim 44 wherein step (B) is performed at isothermal conditions.

50. The method of claim 44 wherein the one or more single-stranded nucleic acid fragments of step (C) have at most 25 nucleotides.

51. The method of claim 44 wherein the one or more single-stranded nucleic acid fragments of step (C) have at most 17 nucleotides.

52. The method of claim 44 wherein the one or more single-stranded nucleic acid fragments of step (C) have at most 12 nucleotides.

53. The method of claim 44 wherein the one or more single-stranded nucleic acid fragments of step (C) have at most 8 nucleotides.

54. The method of claim 44 wherein step (C) is performed at least partially by the use of liquid chromatography.

55. The method of claim 44 wherein step (C) is performed at least partially by the use of mass spectrometry.

56. The method of claim 44 wherein step (C) is performed at least partially by the use of liquid chromatography and mass spectrometry.

57. A method for determining the presence or absence of a target cDNA molecule in a cDNA population, or for determining the presence or absence of a target mRNA in a biological sample, comprising
(A) forming a mixture comprising:
(i) the cDNA molecules in the cDNA population, or the RNA molecules of the biological sample;
(ii) a partially double-stranded nucleic acid probe that comprise:
(a) a sequence of a sense strand of a nicking agent recognition sequence, a sequence of an antisense strand of the nicking agent recognition sequence, or both; and
(b) a 5′ overhang in the strand that either the strand itself or an extension product thereof contains a nicking site nickable by a nicking agent that recognizes the nicking agent recognition sequence, or
a 3′ overhang in the strand that either the strand nor an extension product thereof contains the nicking site,
wherein each overhang comprises a nucleic acid sequence at least substantially complementary to the target cDNA if the target cDNA is single-stranded, to one strand of the target cDNA if the target cDNA is double-stranded, or to the target mRNA;
(B) separating the probe molecules that have hybridized to the cDNA or mRNA molecules from those that have not;
(C) performing an amplification reaction in the presence of the hybridized probe molecules and a nicking agent that recognizes the nicking agent recognition sequence to amplify a single-stranded nucleic acid fragment using one strand of the partially double-stranded nucleic acid probe as a template, if the target cDNA is present in the cDNA population or if the target mRNA is present in the biological sample; and
(D) detecting the presence or absence of the single-stranded nucleic acid fragment of step (C) to determine the presence or absence of the target cDNA in the cDNA population, or to determine the presence or absence of the target mRNA in the biological sample.

58. The method of claim 57 wherein the cDNA molecules in the cDNA population or the RNA molecules of the biological sample are immobilized to a solid support.

59. The method of claim 57 wherein the nicking agent is a nicking endonuclease.

60. A method for determining the presence or absence of a target cDNA molecule in a cDNA population, comprising
(A) forming a mixture of a first oligonucleotide primer (ODNP), a second ODNP, and the cDNA molecules in the cDNA population, wherein
(i) if the target cDNA is a double-stranded nucleic acid having a first strand and a second strand,
the first ODNP comprises a nucleotide sequence of a sense strand of a nicking endonuclease recognition sequence and a nucleotide sequence at least substantially complementary to a first portion of the first strand of the target nucleic acid, and
the second ODNP comprises a nucleotide sequence at least substantially complementary to a second portion of the second strand of the target nucleic acid and comprises a sequence of one strand of a restriction endonuclease recognition sequence, the second portion being located 3′ to the complement of the first portion in the second strand of the target nucleic acid,
or
(ii) if the target nucleic acid is a single-stranded nucleic acid,
the first ODNP comprises a nucleotide sequence of a sense strand of a nicking endonuclease recognition sequence and a nucleotide sequence at least substantially identical to a first portion of the target nucleic acid, and
the second ODNP comprises a nucleotide sequence at least substantially complementary to a second portion of the target nucleic acid and comprises a sequence of one strand of a restriction endonuclease recognition sequence, the second portion being located 5′ to the first portion in the target nucleic acid;
(B) subjecting the mixture to conditions that, if the target cDNA is present in the cDNA population,
(i) extend the first and the second ODNPs to produce an extension product comprising the first ODNP and the second ODNP;
(ii) optionally digesting the extension product of step (i) with a restriction endonuclease that recognizes the restriction endoculease recognition sequence to provide a digestion product;
(iii) amplify a single-stranded nucleic acid fragment using one strand of the extension product of step (B)(i) or the digestion product of step (B)(ii) as a template in the presence of a nicking endonuclease that recognizes the nicking endonuclease recognition sequence; and
(C) detecting the presence or absence of the single-stranded nucleic acid fragment of step (B)(ii) to determine the presence or absence of the target cDNA in the cDNA population.

61. The method of claim 60 wherein the RERS is recognitzable by a type IIs restriction endonuclease.

62. A method for determining the presence or absence of a target cDNA in a cDNA population, comprising
(A) forming a mixture of a first oligonucleotide primer (ODNP), a second ODNP, and the cDNA molecules of the cDNA population, wherein
(i) if the target cDNA is a double-stranded nucleic acid having a first strand and a second strand,
the first ODNP comprises a nucleotide sequence of a sense strand of a first nicking endonuclease recognition sequence (NERS) and a nucleotide sequence at least substantially complementary to a first portion of the first strand of the target cDNA, and
the second ODNP comprises a nucleotide sequence at least substantially complementary to a second portion of the second strand of the target nucleic acid and comprises a sequence of the sense strand of a second NERS, the second portion being located 3′ to the complement of the first portion in the second strand of the target cDNA,
or
(ii) if the target cDNA is a single-stranded nucleic acid,
the first ODNP comprises a nucleotide sequence of a sense strand of a first NERS and a nucleotide sequence at least substantially identical to a first portion of the target cDNA, and
the second ODNP comprises a nucleotide sequence at least substantially complementary to a second portion of the target nucleic acid and comprises a sequence of the sense strand of a second NERS, the second portion being located 5′ to the first portion in the target cDNA;
(B) subjecting the mixture to conditions that, if the target cDNA is present in the cDNA population,
(i) extend the first and the second ODNPs to produce an extension product comprising both the first and the second NERSs;
(ii) amplify a single-stranded nucleic acid fragment using one strand of the extension product of step (B)(i) as a template in the presence of one or more nicking endonucleases (NEs) that recognizes the first and the second NERSs; and
(C) detecting the presence or absence of the single-stranded nucleic acid fragment of step (B)(ii) to determine the presence or absence of the target nucleic acid in the sample.

63. The method of claim 62 wherein the first and second NERSs are identical.

64. The method of claim 62 wherein the first ODNP, the second ODNP or both ODNPs are immobilized to a solid support.

65. A method for determining the presence or absence of a target cDNA in a cDNA population, comprising
(A) forming a mixture of a first oligonucleotide primer (ODNP), a second ODNP, and the cDNA molecules of the cDNA population, wherein
(i) if the target cDNA is a double-stranded nucleic acid having a first strand and a second strand,
the first ODNP comprises a nucleotide sequence of a sense strand of a restriction endonuclease recognition sequence (RERS) and a nucleotide sequence at least substantially complementary to a first portion of the first strand of the target cDNA, and
the second ODNP comprises a nucleotide sequence at least substantially complementary to a second portion of the second strand of the target nucleic acid and comprises a sequence of the sense strand of a second RERS, the second portion being located 3′ to the complement of the first portion in the second strand of the target cDNA,
or
(ii) if the target cDNA is a single-stranded nucleic acid,
the first ODNP comprises a nucleotide sequence of a sense strand of a first RERS and a nucleotide sequence at least substantially identical to a first portion of the target cDNA, and
the second ODNP comprises a nucleotide sequence at least substantially complementary to a second portion of the target nucleic acid and comprises a sequence of the sense strand of a second RERS, the second portion being located 5′ to the first portion in the target cDNA;
(B) subjecting the mixture to conditions that, if the target cDNA is present in the cDNA population,
(i) extend the first and the second ODNPs to produce an extension product comprising both the first and the second RERSs;
(ii) amplify a single-stranded nucleic acid fragment using one strand of the extension product of step (B)(i) as a template in the presence of one more restriction endonucleases (REs) that recognizes the first and the second RERSs; and
(C) detecting the presence or absence of the single-stranded nucleic acid fragment of step (B)(ii) to determine the presence or absence of the target cDNA in the cDNA population.

66. The method of claim 65 wherein the first RERS is identical to the second RERS.

67. The method of claim 65 wherein the first ODNP, the second ODNP, or both ODNPs are immobilized.

68. A method for determining the presence or absence of a target cDNA molecule in a cDNA population, or for determining the presence or absence of a target mRNA molecule in a biological sample, comprising:
(A) forming a mixture comprising:
(i) the cDNA molecules of the cDNA population, or the RNA molecule of the biological sample,
(ii) a first single-stranded template nucleic acid molecule (T1 ) that
(a) comprises one strand of a first nicking agent recognition sequence, and
(b) is at least substantially complementary to the target cDNA if the target cDNA is single-stranded,
is at least substantially complementary to one strand of the target cDNA if the target cDNA is double-stranded, or
is at least substantially complementary to the target mRNA,
(iii) a first nicking agent that recognizes the first nicking agent recognition sequence,
(iv) a DNA polymerase, and
(v) one or more deoxynucleoside triphosphate(s);
(B) maintaining the mixture at conditions that amplify a first single-stranded nucleic acid molecule (A1 ) using a portion of the target cDNA, a portion of the target mRNA, or a portion of the template nucleic acid molecule as a template, if the target cDNA is present in the cDNA population or if the target mRNA is present in the biological sample;
(C) providing a second single-stranded template nucleic acid molecule (T2 ) that is at least substantially complementary to A1 and comprises one strand of a second nicking agent recognition sequence;
(D) performing an amplification reaction in the presence of a second nicking agent that recognizes the second nicking agent recognition sequence to amplify a second single-stranded nucleic acid molecule (A2 ) using either A1 or T2 as a template; and
(E) detecting the presence or absence of A2 to determine the presence or absence of the target cDNA molecule in the cDNA population or the presence or absence of the target mRNA in the biological sample.

69. The method of claim 68 wherein the first template nucleic acid is single-stranded and comprises a sequence, located 3′ to the sequence of one strand of the first nicking agent recognition sequence, that is at least substantially complementary to the 3′ portion of the target cDNA if the target cDNA is single-stranded to one strand of the target cDNA if the target cDNA is double-stranded, or to the target mRNA.

70. The method of claim 68 wherein the target cDNA is double-stranded and comprises the first nicking agent recognition sequence, and wherein the first template nucleic acid comprises the portion of the target cDNA that contains the sequence of the antisense strand of the first nicking agent recognition sequence.

71. The method of claim 68 wherein the target cDNA is single-stranded and comprises the sequence of the sense strand of the first nicking agent recognition sequence, and wherein the first template nucleic acid molecule comprises the sequence of the antisense strand of the first nicking agent recognition sequence.

72. The method of claim 68 wherein the target cDNA is double-stranded and comprises the first nicking agent recognition sequence, and wherein the first template nucleic acid comprises, from 3′ to 55′:
(i) a sequence that is at least substantially complementary to the strand of the target cDNA that comprises the sequence of the sense strand of the first nicking agent recognition sequence,
(ii) the sequence of the antisense strand of the first nicking agent recognition sequence, and
(iii) a sequence that is not substantially complementary to the strand of the target cDNA that comprises the sequence of the sense strand of the first nicking agent recognition sequence.

73. The method of claim 68 wherein the target cDNA is single-stranded and comprises the sequence of the sense strand of the first nicking agent recognition sequence, and wherein the first template nucleic acid comprises, from 3′ to 55′:
(i) a sequence that is at least substantially complementary to the target cDNA,
(ii) the sequence of the antisense strand of the first nicking agent recognition sequence, and
(iii) a sequence that is not substantially complementary to the target cDNA.

74. The method of claim 68 wherein the target cDNA is immobilized.

75. The method of claim 68 wherein T1 comprises the sequence of the sense strand of the first nicking agent recognition sequence.

76. The method of claim 68 wherein T1 comprises the sequence of the antisense strand of the first nicking agent recognition sequence.

77. The method of claim 68 wherein T2 comprises the sequence of the sense strand of the second nicking agent recognition sequence.

78. The method of claim 68 wherein T2 comprises the sequence of the antisense strand of the second nicking agent recognition sequence.

79. The method of claim 75 wherein one or more nucleotides in the sequence of the sense strand of the nicking agent recognition sequence does not form a conventional base pair with nucleotides of the target cDNA or the target mRNA.

80. The method of claim 68 wherein the cDNA molecules or the cDNA population or the RNA molecule fo the biological sample are immobilized to a solid support.

81. The method of claim 68 wherein the template nucleic acid molecule is immobilized to a solid support.

82. The method of claim 76 wherein a sequence located 5′ to the sequence of the antisense strand of the nicking agent recognition sequence is at least substantially identical to a sequence located 5′ to the sequence of the antisense strand of the nicking agent recognition sequence.

83. The method of claim 82 wherein the sequence located 5′ to the sequence of the antisense strand of the nicking agent recognition sequence is exactly identical to the sequence located 5′ to the sequence of the antisense strand of the nicking agent recognition sequence.

84. The method of claim 82 wherein the sequence located 5′ to the sequence of the antisense strand of the nicking agent recognition sequence is at least 10 nucleotides.

85. The method of claim 68 wherein both the first and second nicking agents are a nicking endonuclease.

86. The method of claim 85 wherein the first and second nicking agents are identical.

87. The method of claim 86 wherein the nicking endonuclease is N.BstNB I.

88. The method of 68 wherein both the first and second nicking agents are a restriction endonuclease.

89. The method of 68 wherein steps (A)-(D) are performed in a single vessel.

90. The method of 68 wherein steps (B) and (D) are performed under isothermal conditions.

91. The method of claim 90 wherein steps (B) and (D) are performed at 50° C.-70° C.

92. The method of claim 91 wherein steps (B) and (D) are performed at 60° C.

93. The method of claim 1 wherein steps (B) and (D) are performed in the presence of a 5′→3′ exonuclease deficient DNA polymerase.

94. The method of claim 93 wherein the 5′→3′ exonuclease deficient DNA polymerase is selected from the group consisting of exo⁻Vent, exo⁻Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9°Nm™ DNA polymerase and T4 DNA polymerase homoenzyme.

95. The method of claim 94 wherein the 5′→3′ exonuclease deficient DNA polymerase is exo−Bst polymerase, exo⁻ Bca polymerase, exo⁻ Vent polymerase, 9°Nm™DNA polymerase or exo⁻ Deep Vent polymerase.

96. The method of claim 68 wherein steps (B) and (D) are performed in the presence of a DNA polymerase that has a strand displacement activity.

97. The method of claim 68 wherein steps (B) and (D) are performed in the presence of a DNA polymerase that does not have a strand displacement activity.

98. The method of claim 68 wherein steps (B) and (D) are performed in the presence of a strand displacement facilitator.

99. The method of claim 98 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

100. The method of claim 99 wherein the strand displacement facilitator is trehalose.

101. The method of claim 68 wherein A1 has at most 24 nucleotides.

102. The method of claim 68 wherein A1 has at most 20 nucleotides.

103. The method of claim 68 wherein A1 has at most 17 nucleotides.

104. The method of claim 68 wherein A1 has at most 12 nucleotides.

105. The method of claim 68 wherein A1 has at most 8 nucleotides.

106. The method of claim 68 wherein A2 has at most 24 nucleotides.

107. The method of claim 68 wherein A1 has at most 20 nucleotides.

108. The method of claim 68 wherein A1 has at most 17 nucleotides.

109. The method of claim 68 wherein A1 has at most 12 nucleotides.

110. The method of claim 68 wherein A1 has at most 8 nucleotides.

111. The method of claim 68 wherein step (E) is performed at least partially by the use of a technology selected from the group consisting of mass spectrometry, liquid chromatography, fluorescence polarization, nucleic acid hybridization, and electrophoresis.

112. The method of claim 68 wherein step (E) is performed at least partially by the use of liquid chromatography.

113. The method of claim 68 wherein step (E) is performed at least partially by the use of mass spectrometry.

114. The method of claim 68 wherein step (E) is performed at least partially by the use of liquid chromatography and mass spectrometry.

115. A method for determining the presence or absence of a target cDNA molecule in a cDNA population, comprising:
(A) forming a mixture comprising:
(i) the cDNA molecules of the cDNA population,
(ii) a first single-stranded template nucleic acid molecule (T1 ) that
(a) comprises a sequence of the antisense strand of a first nicking agent recognition sequence, and
(b) is at least substantially complementary to the target cDNA if the target cDNA is single-stranded, or
is at least substantially complementary to one strand of the target cDNA if the target cDNA is double-stranded,
(iii) a second single-stranded template nucleic acid molecule (T2 ) that comprises, from 3′ to 55′:
(a) a sequence that is at least substantially identical to the sequence of the T1 located 5′ to the sequence of the antisense strand of the first nicking agent recognition sequence, and
(b) a sequence of the antisense strand of a second nicking agent recognition sequence,
(iv) a first nicking agent that recognizes the first nicking agent recognition sequence,
(v) a second nicking agent that recognizes the second nicking agent recognition sequence,
(vi) a DNA polymerase, and
(vii) one or more deoxynucleoside triphosphate(s);
(B) maintaining the mixture at conditions that amplify a first single-stranded nucleic acid molecule (A2 ) using the T2 as a template, if the target cDNA is present in the cDNA population; and
(C) detecting the presence or absence of A2 to determine the presence or absence of the target cDNA molecule in the cDNA population.

116. The method of claim 115 wherein the first nicking agent recognition sequence is identical to the second nicking agent recognition sequence.

117. The method of claim 115 wherein T2 comprises a sequence located 3′ to the antisense strand of the second nicking agent recognition sequence that is at least substantially identical to a sequence located 5′ to the antisense strand of the second nicking agent recognition sequence.

118. The method of claim 115 wherein the sequence located 3′ to the antisense strand of the second nicking agent recognition sequence is exactly identical to the sequence located 5′ to the antisense strand of the second nicking agent recognition sequence.

119. The method of claim 115 wherein the cDNA molecule of the cDNA population are immobilized.

120. The method of claim 115 wherein the T1 is immobilized.

121. The method of claim 115 wherein the T2 is immobilized.

122. The method of claim 115 wherein the T2 comprises a sequence located 5′ to the sequence of the antisense strand of the second nicking agent recognition sequence that is at least substantially identical to a sequence located 3′ to the sequence of the antisense strand of the second nicking agent recognition sequence.

123. The method of claim 122 whether the sequence located 5′ to the sequence of the antisense strand of the second nicking agent recognition sequence is at most 10 nucleotides in length.

124. A method for determining the presence or absence of a target cDNA molecule in a cDNA population, comprising:
(A) forming a mixture comprising:
(i) the cDNA molecules of the cDNA population,
(ii) a first single-stranded template nucleic acid molecule (T1 ) that
(a) comprises a sequence of the sense strand of a first nicking agent recognition sequence, and
(b) is at least substantially complementary to the target cDNA if the target cDNA is single-stranded, or
is at least substantially complementary to one strand of the target cDNA if the target cDNA is double-stranded,
(iii) a second single-stranded template nucleic acid molecule (T2 ) that comprises, from 3′ to 55′:
(a) a sequence that is at least substantially complementary to the sequence of T1 located 3′ to the sequence of the sense strand of the first nicking agent recognition sequence, and
(b) a sequence of the antisense strand of a second nicking agent recognition sequence,
(iv) a first nicking agent that recognizes the first nicking agent recognition sequence,
(V) a second nicking agent that recognizes the second nicking agent recognition sequence,
(vi) a DNA polymerase, and
(vii) one or more deoxynucleoside triphosphate(s);
(B) maintaining the mixture at conditions that amplify a first single-stranded nucleic acid molecule (A2 ) using T2 as a template, if the target cDNA is present in the cDNA population; and
(C) detecting the presence or absence of A2 to determine the presence or absence of the target cDNA molecule in the cDNA population.

125. The method of claim 124 wherein the first nicking agent recognition sequence is identical to the second nicking agent recognition sequence.

126. The method of claim 124 wherein the cDNA molecules of the cDNA population are immobilized.

127. The method of claim 124 wherein the T1 is immobilized.

128. The method of claim 124 wherein the T2 is immobilized.

129. A method for determining the presence or absence of a target cDNA molecule in a cDNA population, comprising:
(A) forming a mixture comprising:
(i) the cDNA molecules of the cDNA population,
(ii) a first single-stranded template nucleic acid molecule (T1 ) that
(a) comprises a sequence of the antisense strand of a first nicking agent recognition sequence, and
(b) is at least substantially complementary to the target cDNA if the target cDNA is single-stranded, or
is at least substantially complementary to one strand of the target cDNA if the target cDNA is double-stranded,
(iii) a second single-stranded template nucleic acid molecule (T2 ) that comprises, from 3′ to 55′:
(a) a sequence that is at least substantially identical to the sequence of T1 located 5′ to the sequence of the antisense strand of the first nicking agent recognition sequence, and
(b) a sequence of the sense strand of a second nicking agent recognition sequence,
(iv) a first nicking agent that recognizes the first nicking agent recognition sequence,
(V) a second nicking agent that recognizes the second nicking agent recognition sequence,
(vi) a DNA polymerase, and
(vii) one or more deoxynucleoside triphosphate(s);
(B) maintaining the mixture at conditions that amplify a first single-stranded nucleic acid molecule (A2 ) that is at least substantially identical to the sequence of T1 located 5′ to the antisense strand of the first nicking agent recognition sequence, if the target cDNA is present in the cDNA population; and
(C) detecting the presence or absence of A2 to determine the presence or absence of the target cDNA molecule in the cDNA population.

130. The method of claim 129 wherein the first nicking agent recognition sequence is identical to the second nicking agent recognition sequence.

131. The method of claim 129 wherein the cDNA molecules of the cDNA population are immobilized.

132. The method of claim 129 wherein the T1 is immobilized.

133. The method of claim 129 wherein the T2 is immobilized.

134. A method for determining the presence or absence of a target cDNA molecule in a cDNA population, comprising:
(A) forming a mixture comprising:
(i) the cDNA molecules of the cDNA population,
(ii) a first single-stranded template nucleic acid molecule (T1 ) that
(a) comprises a sequence of the sense strand of a first nicking agent recognition sequence, and
(b) is at least substantially complementary to the target cDNA if the target cDNA is single-stranded, or
is at least substantially complementary to one strand of the target cDNA if the target cDNA is double-stranded,
(iii) a second single-stranded template nucleic acid molecule (T2 ) that comprises, from 3′ to 55′:
(a) a sequence that is at least substantially complementary to the sequence of T1 located 3′ to the sequence of the sense strand of the first nicking agent recognition sequence, and
(b) a sequence of the sense strand of a second nicking agent recognition sequence,
(iv) a first nicking agent that recognizes the first nicking agent recognition sequence,
(V) a second nicking agent that recognizes the second nicking agent recognition sequence,
(vi) a DNA polymerase, and
(vii) one or more deoxynucleoside triphosphate(s);
(B) maintaining the mixture at conditions that amplify a first single-stranded nucleic acid molecule (A2 ) that is at least substantially identical to the sequence of T1 located 3′ to the sense strand of the first nicking agent recognition sequence, if the target cDNA is present in the cDNA population; and
(C) detecting the presence or absence of A2 to determine the presence or absence of the target cDNA molecule in the cDNA population.

135. The method of claim 134 wherein the first nicking agent recognition sequence is identical to the second nicking agent recognition sequence.

136. The method of claim 134 wherein the cDNA molecules of the cDNA population are immobilized.

137. The method of claim 134 wherein the T1 is immobilized.

138. The method of claim 134 wherein the T2 is immobilized.

139. A method for determining the presence or absence of a target cDNA molecule in a cDNA population, or for determining the presence or absence of a target mRNA molecule in a biological sample, comprising:
(A) forming a mixture comprising:
(i) the cDNA molecules of the cDNA population, or the RNA molecule of the biological sample,
(ii) a first template nucleic acid molecule (T1 ) that comprises, from 3′ to 55′:
(a) a first sequence that is at least substantially complementary to the 3′ portion of the target cDNA if the target cDNA is single-stranded, or
a first sequence that is at least substantially complementary to the 3′ portion of one strand of the target cDNA if the target cDNA is double-stranded, or
a first sequence that is at least substantially complementary to the 3′ portion of the target mRNA,
(b) a sequence of the antisense strand of a first nicking agent recognition sequence, and
(c) a second sequence,
(iii) a second template nucleic acid molecule (T2 ) comprising, from 3′ to 55′:
(a) a first sequence that is at least substantially identical to the second sequence of T1 ,
(b) a sequence of the antisense strand of a second nicking agent recognition sequence, and
(c) a second sequence,
(iv) a first nicking agent that recognizes the first nicking agent recognition sequence,
(v) a second nicking agent that recognizes the second nicking agent recognition sequence,
(vi) a DNA polymerase, and
(vii) one or more deoxynucleoside triphosphate(s);
(B) maintaining the mixture at conditions that amplify a single-stranded nucleic acid molecule (A2 ) using the second sequence of T2 as a template, if the target cDNA is present in the cDNA population; and
(C) detecting the presence or absence of A2 to determine the presence or absence of the target cDNA molecule in the cDNA population or the presence or absence of the target mRNA in the biological sample.

140. The method of claim 139 wherein the second sequence of T2 is at least substantially identical to the first sequence of T2 .

141. The method of claim 139 wherein the second sequence of T2 is exactly identical to the first sequence of T2 .

142. The method of claim 139 wherein the first and second nicking agents are identical.

143. The method of claim 142 wherein the first and second nicking agents are a nicking endonuclease.

144. The method of claim 143 wherein the first and the second nicking agents are N.BstNB I.

145. A method for determining the presence or absence of a target cDNA molecule that comprises a sequence of a sense strand of a first nicking agent recognition sequence in a cDNA population, comprising:
(A) forming a mixture comprising:
(i) the cDNA molecules of the cDNA population,
(ii) a first template nucleic acid molecule (T1 ) that comprises, from 3′ to 55′:
(a) a first sequence that is at least substantially complementary to the portion of the target cDNA located immediately 5′ to the sequence of the sense strand of the first nicking agent recognition sequence,
(b) a sequence of the antisense strand of a first nicking agent recognition sequence, and
(c) a second sequence,
(iii) a second template nucleic acid molecule (T2 ) comprising, from 3′ to 55′:
(a) a first sequence that is at least substantially identical to the second sequence of T1 ,
(b) a sequence of the antisense strand of a second nicking agent recognition sequence, and
(c) a second sequence,
(iv) a first nicking agent that recognizes the first nicking agent recognition sequence,
(v) a second nicking agent that recognizes the second nicking agent recognition sequence,
(vi) a DNA polymerase, and
(vii) one or more deoxynucleoside triphosphate(s);
(B) maintaining the mixture at conditions that amplify a single-stranded nucleic acid molecule (A2 ) using the second sequence of T2 as a template, if the target cDNA is present in the cDNA population; and
(C) detecting the presence or absence of A2 to determine the presence or absence of the target cDNA molecule in the cDNA population.

146. The method of claim 145 wherein the second sequence of T2 is at least substantially identical to the first sequence of T2 .

147. The method of claim 145 wherein the second sequence of T2 is exactly identical to the first sequence of T2 .

148. The method of claim 145 wherein the first and second nicking agents are identical.

149. The method of claim 148 wherein the first and second nicking agents are a nicking endonuclease.

150. The method of claim 149 wherein the first and the second nicking agents are N.BstNB I.

151. The method of claim 145 wherein the first sequence of T1 is exactly complementary to the portion of the target cDNA located immediately 5′ to the sequence of the sense strand of the first nicking agent recognition sequence.

152. The method of claim 145 or claim 151 wherein the second sequence of T1 is at least substantially complementary to the portion of the target cDNA located immediately 3′ to the sequence of the sense strand of the first nicking agent recognition sequence.

153. The method of claim 152 wherein the second sequence of T1 is exactly complementary to the portion of the target cDNA located immediately 3′ to the sequence of the sense strand of the first nicking agent recognition sequence.

154. A nucleic acid comprising a sequence that is at least substantially identical to a portion of a naturally occurring genomic DNA or a cDNA of a naturally occurring mRNA, wherein
(A) the portion of the naturally occurring genomic DNA or the cDNA of the naturally occurring mRNA consists of, from 3′ to 55′:
(i) a first sequence that is 3-50 nucleotides in length,
(ii) a sequence of the antisense strand of a nicking agent recognition sequence, and
(iii) a second sequence that is 8-50 nucleotides in length.
(B) the nucleic acid is at most 120 nucleotides in length; and
(C) the nucleic acid comprises sequence A(ii).

155. The nucleic acid of claim 154 wherein sequence A(i) is 5-10 nucleotides in length.

156. The nucleic acid of claim 154 or claim 155 wherein sequence A(iii) is 12-24 nucleotides in length.

157. The nucleic acid of claim 154 wherein the nicking agent recognition sequence is recognizable by a nicking endonuclease.

158. The nucleic acid of claim 157 wherein the nicking endonuclease is N.BstNB I.

159. The nucleic acid of claim 154 wherein the nucleic acid comprises a portion of a naturally occurring genomic DNA.

160. The nucleic acid of claim 154 wherein the nucleic acid comprises a portion of a cDNA of a naturally occurring mRNA.

161. The nucleic acid of claim 154 wherein the nucleic acid is at most 30 nucleotides in length.

162. The nucleic acid of claim 154 wherein the nucleic acid is at most 25 nucleotides in length.

163. The nucleic acid of claim 154 wherein the nucleic acid is at most 20 nucleotides in length.

164. The nucleic acid of claim 154 wherein the nucleic acid is immobilized via its 3′ or 5′ terminus.

165. The nucleic acid of claim 154 wherein the sequence that is at least substantially identical to the portion of the naturally occurring genomic DNA or the cDNA of the naturally occurring mRNA is exactly identical to the portion of the naturally occurring genomic DNA or the cDNA of the naturally occurring mRNA.

166. The nucleic acid of claim 154 wherein the sequence that is at least substantially identical to the portion of the naturally occurring genomic DNA or the cDNA of the naturally occurring mRNA is at least 95% identical to the portion of the naturally occurring genomic DNA or the cDNA of the naturally occurring mRNA.

167. The nucleic acid of claim 154 wherein the sequence that is at least substantially identical to the portion of the naturally occurring genomic DNA or the cDNA of the naturally occurring mRNA is at least 98% identical to the portion of the naturally occurring genomic DNA or the cDNA of the naturally occurring mRNA.

168. A single-stranded nucleic acid that
(a) is at most 100 nucleotides in length,
(b) comprises a sequence of the antisense strand of a nicking agent recognition sequence,
(c) is substantially complementary to a cDNA molecule, and
(d) is capable of functioning as a template to amplify a single-stranded nucleic acid fragment in the presence of a nicking agent that recognizes the nicking agent recognition sequence.

169. A single-stranded nucleic acid that
(a) is at most 100 nucleotides in length,
(b) comprises a sequence of the sense strand of a nicking agent recognition sequence,
(c) is substantially complementary to a cDNA molecule, and
(d) when annealing to the cDNA molecule, allows for the amplification of a portion of the cDNA molecule in the presence of a nicking agent that recognizes the nicking agent recognition sequence.

170. The single-stranded nucleic acid of claim 168 or claim 169 wherein the single-stranded nucleic acid is at most 50 nucleotides in length.

171. The single-stranded nucleic acid of claim 168 or claim 169 wherein the single-stranded nucleic acid is at most 30 nucleotides in length.

172. The single-stranded nucleic acid of claim 168 or claim 169 wherein the single-stranded nucleic acid is at most 25 nucleotides in length.

173. The single-stranded nucleic acid of claim 168 or claim 169 wherein the single-stranded nucleic acid is at most 20 nucleotides in length.

174. The single-stranded nucleic acid of claim 168 or claim 169 wherein the nicking agent is a nicking endonuclease.

175. The single-stranded nucleic acid of claim 174 wherein the nicking endonuclease is N.BstNB I.

176. The single-stranded nucleic acid of claim 168 or claim 169 wherein the single-stranded nucleic acid is exactly identical to a portion of the cDNA molecule.

177. The single-stranded nucleic acid of claim 168 or claim 169 wherein the single-stranded nucleic acid is immobilized to a solid support.

178. A method for determining the presence or absence of a target cDNA molecule in a cDNA population, comprising:
(A) forming a mixture comprising:
(i) the cDNA molecules of the cDNA population;
(ii) an oligonucleotide primer that
(a) comprises a sequence of the sense strand of a double-stranded nicking agent recognition sequence recognizable by a nicking agent that nicks outside the recognition sequence, and
(b) is at least substantially complementary to a first region of the single-stranded target nucleic acid or of one strand of the double-stranded target nucleic acid; and
(iii) a partially double-stranded nucleic acid that
(a) comprises a double-stranded type IIs restriction endonucelase recognition sequence, and
(b) a 3′ overhang that is at least substantially complementary to a second region of the single-stranded target cDNA or of the one strand of the double-stranded target cDNA located 5′ to the first region the single-stranded target cDNA or of the one strand of the double-stranded target cDNA;
under conditions that allow for hybridization between the oligonucleotide primer and the first region of the single-stranded target cDNA or of the one strand of the double-stranded nucleic acid and between the 3′ overhang of the partially double-stranded nucleic acid and the second region of the single-stranded target cDNA or of the one strand of the double-stranded nucleic acid;
(B) digesting the single-stranded target cDNA or the one strand of the double-stranded target cDNA that have hybridized to the oligonucleotide primer and to the partially double-stranded nucleic acid in the second region.
(C) performing an amplification reaction that amplify a single-stranded nucleic acid molecule using a portion of the single-stranded target cDNA or of the one strand of the double-stranded target cDNA digested in step (B) as a template in the presence of the nicking agent, and
(D) detecting the presence or absence of the single-stranded nucleic acid molecule of step (C) to determine the presence or absence of the target cDNA molecule in the cDNA population.

179. The method of claim 178 wherein the double-stranded nicking agent recognition sequence is recognizable by N.BstNB I.

180. The method of claim 178 wherein a nucleotide in the sequence of the sense strand of the double-stranded nicking agent recognition sequence does not form a conventional base pair with another nucleotide of the single-stranded target cDNA or the one strand of the double-stranded cDNA when the oligonucleotide primer anneals to the target nucleic acid.

181. The method of claim 178 wherein the TRERS is recognizably by PleI or MlyI.
